SEQUENCE LISTING (26 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 3 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 4 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 6 - protein, 61 aa, Severe acute respiratory syndrome coronavirus 2
MFHLVDFQVTIAEILLIIMRTFKVSIWNLDYIINLIIKNLSKSLTENKYSQLDEEQPMEID

SEQ ID NO: 7 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 8 - protein, 43 aa, Severe acute respiratory syndrome coronavirus 2
MIELSLIDFYLCFLAFLLFLVLIMLIIFWFSLELQDHNETCHA

SEQ ID NO: 9 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 10 - protein, 38 aa, Severe acute respiratory syndrome coronavirus 2
MGYINVFAFPFTIYSLLLCRMNSRNYIAQVDVVNFNLT

SEQ ID NO: 11 - protein, 330 aa, Homo sapiens [sequence omitted]

SEQ ID NO: 12 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 13 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 14 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 428 aa, Homo sapiens
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 16 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 17 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 18 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 19 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 20 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 22 - protein, 8 aa, Artificial Sequence
  Synthetic Construct
  MISC_FEATURE (2)..(2): Xaa is Val or Ile
  MISC_FEATURE (4)..(4): Xaa can be any naturally occurring amino acid
DXEXNPGP

SEQ ID NO: 23 - DNA, 30 bp, Artificial Sequence
  Synthetic Construct
gtttttcggctattcccaatagccgttttg

SEQ ID NO: 24 - DNA, 1229 bp, Artificial Sequence
  Synthetic Construct
cgcggatcctaatacgactcactatagggaatagccgaaaaacaaaaaacaaaaaaaacaaaaaaaaaaccaaaaaaacaaaacacaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacggctatt

SEQ ID NO: 25 - RNA, 96 nt, Hepatitis D virus
ggcucaucucgacaagaggcggcaguccucaguacucuuacucuuuucuguaaagaggagacugcuggacucgccgcccaaguucgagcaugagcc

SEQ ID NO: 26 - RNA, 74 nt, Hepatitis D virus
ggcuagaggcggcaguccucaguacucuuacucuuuucuguaaagaggagacugcuggacucgccgcccgagcc

(19) United States
(12) Patent Application Publication
(10) Pub. No.: US 2023/0203192 A1
(43) Pub. Date: Jun. 29, 2023

(54) COMPOSITIONS AND METHODS FOR PRODUCING HUMAN POLYCLONAL ANTIBODIES

(71) Applicant: FLAGSHIP PIONEERING, INC., Cambridge, MA (US)

(72) Inventors: Alexandra Sophie DE BOER, Somerville, MA (US); Avak KAHVEJIAN, Lexington, MA (US); Frances Marion Nicole ANASTASSACOS, Boston, MA (US); Jennifer A. NELSON, Brookline, MA (US); Nicholas McCartney PLUGIS, Duxbury, MA (US); Roger Joseph HAJJAR, Lexington, MA (US); Yann Paul Guy Régis ECHELARD, Jamaica Plain, MA (US)

(21) Appl. No.: 17/926,254

(22) PCT Filed: May 20, 2021
(86) PCT No.: PCT/US2021/033449

§ 371 Date: Nov. 18, 2022

Related U.S. Application Data
(60) Provisional application No. 63/125,717, filed on Dec. 15, 2020.
(60) Provisional application No. 63/074,416, filed on Sep. 3, 2020.
(60) Provisional application No. 63/038,072, filed on Jun. 11, 2020.
(60) Provisional application No. 63/027,931, filed on May 20, 2020.

(51) Int. Cl. C07K 16/32; C07K 16/08; A61P 31/14; A61P 35/00

(52) U.S. Cl. CPC C07K 16/32; C07K 16/08; A61P 31/14; A61P 35/00; C07K 2317/76

(57) ABSTRACT

The disclosure provides compositions and methods for generating polyclonal antibodies, for example, using circular polyribonucleotides and non-human animals having humanized immune systems.

### SEQUENCE LISTING

The instant application contains a Sequence Listing which has been submitted electronically in ASCII format and is hereby incorporated by reference in its entirety. Said ASCII copy, created on May 20, 2021 is named 51509-021WO5_Sequence_Listing_5.20.21_ST25 and is 57,710 bytes in size.

### BACKGROUND

There is a need for therapeutics against disease and pathogenic agents, such as viruses.

### SUMMARY

The disclosure relates generally to compositions and methods for making and using polyclonal antibodies against a target, e.g., a pathogen, a cancer, or a toxin. The compositions include, and the methods use, a circular polyribonucleotide that includes a sequence that encodes an antigen (e.g., an epitope) of the target. The produced polyclonal antibodies can be used to treat a disease (e.g., in a human subject) caused by the pathogen or the cancer expressing the antigen. The produced polyclonal antibodies can be used to treat a condition (e.g., in a human subject) associated with a toxin comprising the antigen.

In one aspect, the invention features a method of inducing an immune response to a target (e.g., a microorganism, a cancer, or toxin) in a non-human animal capable of producing human antibodies. The method includes administering a composition (e.g., an immunogenic composition) comprising a circular RNA that comprises a sequence encoding a target antigen to the non-human animal.

In some embodiments, the antigen comprises a sequence having at least about 80%, 85%, 90%, 95%, 97%, 98%, 99%, or 100% sequence identity to a sequence from TABLE 1. In some embodiments, the antigen is a microorganism antigen. In some embodiments, the antigen is from a pathogenic microorganism. In some embodiments, the antigen is from a virus or a fragment thereof, from a bacterium or a fragment thereof, from a eukaryotic parasite or a fragment thereof, or from a fungus or a fragment thereof In some embodiments, the antigen is from a DNA virus or a fragment thereof, a positive strand RNA virus or a fragment thereof, or a negative strand RNA virus or a fragment thereof. In some embodiments, the antigen is from a virus selected from a group consisting of Marburg, ebola, rabies, HIV, smallpox, hantavirus, dengue, rotavirus, Crimean-Congo hemorrhagic fever, lassa fever, nipha and henipaviral disease, rift valley fever, plague, tularemia, machupo, typhus fever, CMV, Hepatitis B, Hepatitis C, HSV, parvovirus B19, rubella, zika, chickenpox, RSV, Para influenza, rhinovirus, adenovirus, metapneumovirus, bocavirus, community acquired respiratory virus, measles, mumps, and varicella, or any fragment thereof In some embodiments, the antigen is selected from a coronavirus or a fragment thereof, a betacoronavirus or a fragment thereof, or a sarbecovirus or a fragment thereof. In some embodiments, the antigen is from severe acute respiratory syndrome-related coronavirus or a fragment thereof, a merbecovirus or a fragment thereof, or Middle East respiratory syndrome coronavirus (MERS-CoV) or a fragment thereof. In some embodiments, the antigen is from severe acute respiratory syndrome coronavirus 2 (SARS-CoV-2) or a fragment thereof or severe acute respiratory syndrome coronavirus 1 (SARS-CoV-1) or a fragment thereof In some embodiments, the antigen is from a membrane protein of a virus or a variant or fragment thereof, an envelope protein of a virus or a variant or fragment thereof, a spike protein of a virus or a variant or fragment thereof, a receptor binding domain of a spike protein of a virus or a variant or fragment thereof, a nucleocapsid protein of a virus or a variant or fragment thereof, an accessory protein of a virus or a variant or fragment thereof. In some embodiments, the spike protein lacks a cleavage site. In some embodiments, an accessory protein of a virus is selected from a group consisting of ORF3a, ORF7a, ORF7b, ORF8, ORF10, or any fragment thereof In some embodiments, the antigen is a variant of an accessory protein of a virus selected from a group consisting of ORF3a, ORF7a, ORF7b, ORF8, ORF10, or any fragment thereof.

In some embodiments, the antigen is from a bacterium selected from a group consisting of Group B strep, toxoplasma, and syphilis, or any fragment thereof.

In some embodiments, the antigen is a cancer antigen, e.g., HER2 or a cancer neoantigen.

In some embodiments, the antigen is a toxin antigen, e.g., a mycotoxin, cytotoxin, or neurotoxin (e.g., a toxin in venom, from a toxic plant or fungus, or from a drug).

In some embodiments, the circular polyribonucleotide comprises a plurality of sequences, each of the plurality encoding a different antigen. In some embodiments, the circular polyribonucleotide comprises two or more ORFs. In some embodiments, the circular polyribonucleotide comprises a sequence encoding at least 2, 3, 4 or 5 antigens. In some embodiments, the circular polyribonucleotide comprises at least five ORFs. In some embodiments, the circular polyribonucleotide comprises a sequence encoding antigens from at least two different microorganisms. In some embodiments, the circular polyribonucleotide comprises a sequence encoding antigens from at least two different tumors or cancers. In some embodiments, the circular polyribonucleotide comprises a sequence encoding antigens from at least two different toxins. In some embodiments, the antigen comprises an epitope. In some embodiments, the antigen comprises an epitope recognized by a B cell. In some embodiments, the antigen comprises at least two epitopes.

In some embodiments, the composition further comprises a second circular polyribonucleotide comprising a sequence encoding a second antigen. In some embodiments, the composition further comprises a second circular polyribonucleotide comprising a second ORF. In some embodiments, the composition further comprises a third, fourth, or fifth circular polyribonucleotide comprising a sequence encoding a third, fourth, or fifth antigen. In some embodiments, the first antigen, second antigen, third antigen, fourth antigen, and fifth antigen are different antigens (e.g., different antigens from the same target or from different targets, e.g., from different target pathogens).

In some embodiments, the non-human animal is a mammal that has been genetically modified to express human immunoglobulins, e.g., it has a humanized immune system. In some embodiments, the non-human animal having a humanized immune system is an ungulate (e.g., a pig, cow, sheep, horse). In some embodiments, the non-human animal having a humanized immune system is a transchromosomal ungulate. In some embodiments, the non-human animal having a humanized immune system is a cow. In some embodiments, the non-human animal having a humanized immune system is a goat or chicken. In some embodiments, the non-human animal (e.g., cow) having a humanized immune system comprises a human artificial chromosome (HAC) vector that comprises the humanized immunoglobulin gene locus. In some embodiments, the humanized immunoglobulin gene locus encodes an immunoglobulin heavy chain. In some embodiments, the immunoglobulin heavy chain comprises an IgG isotype heavy chain. In some embodiments, the immunoglobulin heavy chain comprises an IgG1, IgG2, IgG3, or IgG4 isotype heavy chain.

In some embodiments, the non-human animal having a humanized immune system comprises a B cell having a humanized B cell receptor, the humanized B cell receptor binds to the antigen. In some embodiments, the non-human animal having a humanized immune system comprises a plurality of B cells comprising a first B cell that binds to a first epitope of the antigen and a second B cell that binds to a second epitope of the antigen.

In some embodiments, the non-human animal having a humanized immune system comprises a T cell, wherein the T cell comprises a T Cell Receptor that binds to the antigen. In some embodiments, upon activation, the T cell enhances production of an antibody that that binds to the antigen. In some embodiments, upon activation, the T cell enhances antibody production by a B cell that binds to the antigen. In some embodiments, upon activation, the T cell enhances survival, proliferation, plasma cell differentiation, somatic hypermutation, immunoglobulin class switching, or a combination thereof of a B cell that that binds to the antigen.

In some embodiments, the non-human animal having the humanized immune system produces an antibody that specifically binds to the antigen. In some embodiments, the non-human animal having the humanized immune system produces polyclonal antibodies wherein an antibody of the polyclonal antibodies specifically binds to the antigen. In some embodiments, the antibody is a humanized antibody or a fully human antibody. In some embodiments, the antibody is antibody an IgG isotype antibody. In some embodiments, the antibody is an IgG1, IgG2, IgG3, or IgG4 isotype antibody. In some embodiments, the antibody is an IgA isotype antibody. In some embodiments, the antibody is an IgM isotype antibody. In some embodiments, the non-human animal comprises plurality of polyclonal antibodies that specifically bind at least two epitopes that are encoded by the circular polyribonucleotide. In some embodiments, the plurality of polyclonal antibodies comprises humanized antibodies or fully human antibodies. In some embodiments, the plurality of polyclonal antibodies comprises IgG antibodies, IgG1 antibodies, IgG2 antibodies, IgG3 antibodies, IgG4 antibodies, IgM antibodies, IgA antibodies, or a combination thereof. In some embodiments, the humanized immunoglobulin gene locus encodes an immunoglobulin light chain. In some embodiments, the immunoglobulin light chain comprises a kappa light chain or a lambda light chain

In some embodiments, the composition further comprises a pharmaceutically acceptable carrier or excipient. In some embodiments, the composition further comprises a pharmaceutically acceptable excipient and is free of any carrier. In some embodiments, the composition is formulated with a carrier, e.g., a lipid nanoparticle.

In some embodiments, the composition further comprises an adjuvant (e.g., Addavax™ adjuvant, MFS9® adjuvant, AS03, complete Freund's adjuvant). In some embodiments, the composition further comprises protamine.

In some embodiments, the method further comprises administering an adjuvant (e.g., an adjuvant described herein) to the non-human animal. The adjuvant may be administered in the same or a separate composition as the composition comprising the circular polyribonucleotide.

In some embodiments, the method further comprises administering protamine to the non-human animal having a humanized immune system.

In some embodiments, the method further comprises administering the circular polyribonucleotide at least two times to the non-human animal having a humanized immune system to generate hyperimmune plasma.

In some embodiments, the method further comprises administering a second agent or vaccine to the non-human animal having a humanized immune system. In some embodiments, the vaccine is pneumococcal polysaccharide vaccine (e.g., PCV13 or PPSV23). In some embodiments, the vaccine is for a bacterial infection. In some embodiments, the non-human animal is inoculated with the circular RNA by injection.

In some embodiments, the method further comprises pre-administering (priming) the non-human animal with an agent to improve immunogenic response. For example, the method includes administering the protein antigen to the non-human animal prior (e.g., between 1-7 days, e.g., 1, 2, 3, 4, 5, 6, 7 days prior) to administration of the circular RNA comprising a sequence encoding the antigen. The protein antigen may be administered as a protein preparation, or encoded in a plasmid (pDNA), or presented in a virus-like-particle (VLP), formulated in a lipid nanoparticle (LNP), or the like.

In some embodiments, the method further comprises collecting blood or plasma from the non-human animal having a humanized immune system. In some embodiments, the method further comprises purifying polyclonal antibodies from the non-human animal having a humanized immune system. In embodiments, the method further comprises collecting blood from the immunized non-human animal and/or purifying antibodies against the antigen from the blood.

In embodiments, the method further comprises evaluating the non-human mammal for antibody response to the antigen, e.g., before and/or after the administration.

In another aspect, the invention features a method of producing a human polyclonal antibody preparation against a target (e.g., a pathogen, a cancer, or a toxin). The method includes (a) administering to a non-human animal capable of producing human antibodies an immunogenic composition comprising a circular RNA that comprises a sequence encoding a target antigen as described above, (b) collecting blood or plasma from the non-human animal, (c) purifying antibodies against the target from the blood or plasma, and (d) formulating the antibodies as a therapeutic or pharmaceutical preparation for human use.

In another aspect, the invention features a method of treating a human subject in need thereof, comprises administering to the human subject a preparation of polyclonal antibodies produced by a non-human animal having a humanized immune system, wherein the non-human animal has been inoculated with a circular polyribonucleotide that comprises a sequence encoding a target antigen sequence described herein. For example, the non-human animal having a humanized immune system has been subject to a method of inducing an immune response to the target as described herein.

In some embodiments, the human subject is at risk for exposure to a disease associated with the target or has been diagnosed with a disease associated with the target. For example, the target is a pathogenic virus or bacteria and the subject is at risk or is diagnosed with a disease or condition caused by the pathogenic virus or bacteria.

In some embodiments, the administration is before, after, or simultaneously with a human subject's risk of exposure to the disease.

In some embodiments, the human subject is at risk for or has been diagnosed with a cancer and the antigen is a cancer antigen.

In some embodiments, the human subject was bitten or stung by a venomous animal, absorbed a toxin, inhaled a toxin, ingested a toxin, or overdosed on a drug and the antigen is a from a toxin.

In another aspect, the invention features a composition comprising (a) a circular polyribonucleotide comprising a sequence encoding an antigen to a target; and (b) a non-human B cell comprising a humanized immunoglobulin gene locus and a humanized B cell receptor, wherein the humanized B cell receptor binds to the antigen.

In one embodiment, the composition comprises a plurality of non-human B cells, wherein a non-human B cell of the plurality comprises a humanized immunoglobulin gene locus, wherein the plurality of B cells comprises a first B cell that binds to a first epitope of the antigen and a second B cell that binds to a second epitope of the antigen.

In one embodiment, the composition comprises a non-human B cell comprising a humanized immunoglobulin gene locus and humanized B cell receptor, wherein the humanized B cell receptor binds to the antigenic sequence.

In another aspect, the invention features a composition comprising (a) a circular polyribonucleotide comprising a sequence encoding an antigen of a target; and (b) plasma from a non-human animal comprising a humanized immune system.

In one embodiment, the composition further comprises the antigen.

Exemplary embodiments of the invention are described in the enumerated paragraphs below.

E1. A method of producing polyclonal antibodies comprising administering a circular polyribonucleotide comprising a sequence encoding an antigen to a non-human animal having a humanized immune system.

E2. A method of producing polyclonal antibodies comprising administering a circular polyribonucleotide comprising an antigenic sequence to a non-human animal having a humanized immune system.

E3. A method of producing polyclonal antibodies comprising immunizing a non-human animal comprising a humanized immune system with a circular polyribonucleotide comprising a sequence encoding an antigen.

E4. A method of producing polyclonal antibodies comprising immunizing a non-human animal comprising a humanized immune system with a circular polyribonucleotide comprising an antigenic sequence.

E5. A method of inducing an immune response to an antigen comprising administering a circular polyribonucleotide comprising a sequence encoding the antigen to a non-human animal comprising a humanized immune system.

E6. A method of inducing an immune response to an antigenic sequence comprising administering a circular polyribonucleotide comprising the antigenic sequence to a non-human animal comprising a humanized immune system.

E7. A method of inducing an immune response to an antigen comprising immunizing a non-human animal comprising a humanized immune system with a circular polyribonucleotide comprising a sequence encoding the antigen.

E8. A method of inducing an immune response to an antigenic sequence comprising immunizing a non-human animal comprising a humanized immune system with a circular polyribonucleotide comprising the antigenic sequence.

E9. The method of any one of the preceding embodiments, wherein the antigen is a microorganism antigen, cancer antigen, or toxin antigen.

E10. The method of any one of the preceding embodiments, wherein the antigen and/or antigenic sequence is from a microorganism, a cancer, or a toxin.

E11. The method of any one of the preceding embodiments, wherein the antigen and/or antigenic sequence is from a pathogenic microorganism.

E12. The method of any one of the preceding embodiments, wherein the antigen and/or antigenic sequence is from a virus or a fragment thereof, from a bacterium or a fragment thereof, from a eukaryotic parasite or a fragment thereof, or from a fungus or a fragment thereof.

E13. The method of any one of the preceding embodiments, wherein the antigen and/or antigenic sequence is from a DNA virus or a fragment thereof, a positive strand RNA virus or a fragment thereof, or a negative strand RNA virus or a fragment thereof.

E14. The method of any one of the preceding embodiments, wherein the antigen and/or antigenic sequence is from a virus selected from a group consisting of Marburg, ebola, rabies, HIV, smallpox, hantavirus, dengue, rotavirus, Crimean-Congo hemorrhagic fever, lassa fever, nipha and henipaviral disease, rift valley fever, plague, tularemia, machupo, typhus fever, CMV, Hepatitis B, Hepatitis C, HSV, parvovirus B19, rubella, zika, chickenpox, RSV, Para influenza, rhinovirus, adenovirus, metapneumovirus, bocavirus, community acquired respiratory virus, measles, mumps, and varicella, or any fragment thereof.

E15. The method of any one of the preceding embodiments, wherein the antigen and/or antigenic sequence is selected from a coronavirus or a fragment thereof, a betacoronavirus or a fragment thereof, or a sarbecovirus or a fragment thereof.

E16. The method of any one of the preceding embodiments, wherein the antigen and/or antigenic sequence is from severe acute respiratory syndrome-related coronavirus or a fragment thereof, a merbecovirus or a fragment thereof, or Middle East respiratory syndrome coronavirus (MERS-CoV) or a fragment thereof.

E17. The method of any one of the preceding embodiments, wherein the antigen and/or antigenic sequence is from severe acute respiratory syndrome coronavirus 2 (SARS-CoV-2) or a fragment thereof or severe acute respiratory syndrome coronavirus 1 (SARS-CoV-1) or a fragment thereof.

E18. The method of any one of the preceding embodiments, wherein the antigen and/or antigenic sequence is from a membrane protein of a virus or a variant or fragment thereof, an envelope protein of a virus or a variant or fragment thereof, a spike protein of a virus or a variant or fragment thereof, a receptor binding domain of a spike protein of a virus or a variant or fragment thereof, a nucleocapsid protein of a virus or a variant or fragment thereof, an accessory protein of a virus or a variant or fragment thereof

E19. The method of embodiment E18, wherein the spike protein lacks a cleavage site.

E20. The method of any one of the preceding embodiments, wherein an accessory protein of a virus is selected from a group consisting of ORF3a, ORF7a, ORF7b, ORFS, ORF10, or any fragment thereof.

E21. The method of any one of the preceding embodiments, wherein the antigen is a variant of an accessory protein of a virus selected from a group consisting of ORF3a, ORF7a, ORF7b, ORF8, ORF10, or any fragment thereof.

E22. The method of any one of the preceding embodiments, wherein the antigen and/or antigenic sequence is from a bacterium selected from a group consisting of Group B strep, toxoplasma, and syphilis, or any fragment thereof.

E23. The method of any one of the preceding embodiments, wherein the cancer antigen is HER2 or a cancer neoantigen.

E24. The method of any one of the preceding embodiments, wherein the toxin antigen is from an animal venom, plant, or fungus.

E25. The method of any one of the preceding embodiments, wherein the toxin antigen is from a drug (e.g., digoxin).

E26. The method of any one of the preceding embodiments, wherein the circular polyribonucleotide comprises a sequence encoding two or more antigens or antigenic sequences.

E27. The method of any one of the preceding embodiments, wherein the circular polyribonucleotide comprises two or more ORFs.

E28. The method of any one of the preceding embodiments, wherein the circular polyribonucleotide comprises a sequence encoding at least 2, 3, 4, or 5 antigens.

E29. The method of any one of the preceding embodiments, wherein the circular polyribonucleotide comprises at least five ORFs.

E30. The method of any one of the preceding embodiments, wherein the circular polyribonucleotide comprises a sequence encoding antigens from at least two different microorganisms.

E31. The method of any one of the preceding embodiments, wherein the antigen comprises an epitope.

E32. The method of any one of the preceding embodiments, wherein the antigen comprises an epitope recognized by a B cell.

E33. The method of any one of the preceding embodiments, wherein the antigen comprises at least two epitopes.

E34. The method of any one of the preceding embodiments, further comprising administering and/or immunizing the non-human animal having the humanized immune system with a second circular polyribonucleotide comprising a sequence encoding a second antigen or a second antigenic sequence.

E35. The method of any one of the preceding embodiments, further comprising administering and/or immunizing the non-human animal having the humanized immune system with a second circular polyribonucleotide comprising a second ORF.

E36. The method of any one of the preceding embodiments, further comprising administering and/or immunizing the non-human animal having the humanized immune system with a third, fourth, or fifth circular polyribonucleotide comprising a sequence encoding a third, fourth, or fifth antigen or a third, fourth, or fifth antigenic sequence.

E37. The method of any one of the preceding embodiments, wherein the first antigen, second antigen, third antigen, fourth antigen, and fifth antigen are different antigens or the first antigenic sequence, second antigenic sequence, third antigenic sequence, fourth antigenic sequence, and fifth antigenic sequence are different antigenic sequences.

E38. The method of any one of the preceding embodiments, wherein the non-human animal having a humanized immune system is a mammal.

E39. The method of any one of the preceding embodiments, wherein the non-human animal having a humanized immune system is an ungulate.

E40. The method of any one of the preceding embodiments, wherein the non-human animal having a humanized immune system is a transchromosomal ungulate.

E41. The method of any one of the preceding embodiments, wherein the non-human animal having a humanized immune system is a cow or bovine.

E42. The method of any one of the preceding embodiments, wherein the non-human animal having a humanized immune system comprises a human artificial chromosome (HAC) vector that comprises the humanized immunoglobulin gene locus.

E43. The method of any one of the preceding embodiments, wherein the humanized immunoglobulin gene locus encodes an immunoglobulin heavy chain.

E44. The method of any one of the preceding embodiments, wherein the humanized immunoglobulin heavy chain comprises an IgG isotype heavy chain.

E45. The method of any one of the preceding embodiments, wherein the humanized immunoglobulin heavy chain comprises an IgG1, IgG2, IgG3, or IgG4 isotype heavy chain.

E46. The method of any one of the preceding embodiments, wherein the humanized immunoglobulin gene locus encodes an immunoglobulin light chain.

E47. The method of any one of the preceding embodiments, wherein the immunoglobulin light chain comprises a kappa light chain or a lambda light chain.

E48. The method of any one of the preceding embodiments, wherein the non-human animal having a humanized immune system comprises a B cell having a humanized B cell receptor, the humanized B cell receptor binds to the antigen.

E49. The method of any one of the preceding embodiments, wherein the non-human animal having a humanized immune system comprises a plurality of B cells comprising a first B cell that binds to a first epitope of the antigen and a second B cell that binds to a second epitope of the antigen.

E50. The method of any one of the preceding embodiments, wherein the non-human animal having a humanized immune system comprises a T cell, wherein the T cell comprises a T Cell Receptor that binds to the antigen.

E51. The method of any one of the preceding embodiments, wherein upon activation, the T cell enhances production of an antibody that that binds to the antigen.

E52. The method of any one of the preceding embodiments, wherein upon activation, the T cell enhances antibody production by a B cell that binds to the antigen.

E53. The method of any one of the preceding embodiments, wherein upon activation, the T cell enhances survival, proliferation, plasma cell differentiation, somatic hypermutation, immunoglobulin class switching, or a combination thereof of a B cell that that binds to the antigen.

E54. The method of any one of the preceding embodiments, wherein an antibody of the polyclonal antibodies specifically binds to the antigen or antigenic sequence.

E55. The method of any one of the preceding embodiments, wherein an antibody of the polyclonal antibodies is a human antibody.

E56. The method of any one of the preceding embodiments, wherein an antibody of the polyclonal antibodies is an IgG isotype antibody.

E57. The method of any one of the preceding embodiments, wherein an antibody of the polyclonal antibodies is an IgG1, IgG2, IgG3, or IgG4 isotype antibody.

E58. The method of any one of the preceding embodiments, wherein an antibody of the polyclonal antibodies is an IgA isotype antibody.

E59. The method of any one of the preceding embodiments, wherein an antibody of the polyclonal antibodies is an IgM isotype antibody.

E60. The method of any one of the preceding embodiments, wherein the polyclonal antibodies specifically bind at least two epitopes that are encoded by the circular polyribonucleotide.

E61. The method of any one of the preceding embodiments, wherein the polyclonal antibodies comprises fully human polyclonal antibodies.

E62. The method of any one of the preceding embodiments, wherein the polyclonal antibodies comprise IgG antibodies, IgG1 antibodies, IgG2 antibodies, IgG3 antibodies, IgG4 antibodies, IgM antibodies, IgA antibodies, or a combination thereof.

E63. The method of any one of the preceding embodiments, wherein the circular polyribonucleotide is formulated with a pharmaceutically acceptable carrier or excipient.

E64. The method of any one of the preceding embodiments, wherein the circular polyribonucleotide is formulated pharmaceutically acceptable excipient and is free of any carrier.

E65. The method of any one of the preceding embodiments, wherein the circular polyribonucleotide is formulated with a carrier.

E66. The method of any one of the preceding embodiments, wherein the circular polyribonucleotide is formulated with a lipid nanoparticle for administration.

E67. The method of any one of the preceding embodiments, wherein the circular polyribonucleotide is formulated with an adjuvant.

E68. The method of any one of the preceding embodiments, wherein the circular polyribonucleotide is further formulated with protamine.

E69. The method of any one of the preceding embodiments, further comprising administering an adjuvant to the non-human animal having a humanized immune system.

E70. The method of any one of the preceding embodiments, further comprising administering the circular polyribonucleotide is formulated with an adjuvant.

E71. The method of any one of the preceding embodiments, wherein the circular polyribonucleotide and an adjuvant are administered in separate compositions.

E72. The method of embodiment E71, wherein the adjuvant is a saponin or an oil emulsion

E73. The method of embodiment E72, wherein the oil emulsion is a squalene-water emulsion (e.g., Addavax™ adjuvant, MF59 or AS03).

E74. The method of any one of the preceding embodiments, further comprising administering protamine to the non-human animal having a humanized immune system.

E75. The method of any one of the preceding embodiments, further comprising administering the circular polyribonucleotide at least two times to the non-human animal having a humanized immune system to generate hyperimmune plasma.

E76. The method of any one of the preceding embodiments, further comprising collecting plasma from the non-human animal having a humanized immune system.

E77. The method of any one of the preceding embodiments, further comprising purifying polyclonal antibodies from the plasma of a non-human animal having a humanized immune system.

E78. The method of any one of the preceding embodiments, further comprising administering a second agent or a vaccine to the non-human animal having a humanized immune system.

E79. The method of any one of the preceding embodiments, wherein the vaccine is pneumococcal polysaccharide vaccine (e.g., PCV13 or PPSV23).

E80. The method of any one of the preceding embodiments, wherein the vaccine is for a bacterial infection.

E81. The method of any one of the preceding embodiments, wherein the non-human animal having a humanized immune system is immunized with the circular polyribonucleotide by injection.

E82. The method of any one of the preceding embodiments, further comprising administering the non-human animal having a humanized immune system with the antigen prior to administration of the circular polyribonucleotide.

E83. The method of any one of the preceding embodiments, further comprising administering the antigen to the non-human animal having a humanized immune system at least 1, 2, 3, 4, 5, 6, or 7 days prior to administering the circular polyribonucleotide.

E84. The method of any one of the preceding embodiments, wherein the antigen is administered as a protein preparation, encoded in a plasmid (pDNA), presented in a virus-like particle (VLP), or formulated in a lipid nanoparticle (LNP).

E85. The method of any one of the preceding embodiments, further comprising evaluating the non-human animal having a humanized immune system for antibody response to the antigen and/or antigenic sequence.

E86. The method of any one of the preceding embodiments, wherein the evaluating is prior to administration of the circular polyribonucleotide and/or after the administration of the circular polyribonucleotide.

E87. A method of producing a human polyclonal antibody preparation against a target, comprising:

a) administering to a non-human animal capable of producing human antibodies an immunogenic composition comprising a circular polyribonucleotide that comprises a sequence encoding an antigen of the target,

b) collecting blood or plasma from the non-human animal capable of producing human antibodies,

c) purifying antibodies against the antigen from the blood or plasma, and

d) formulating the antibodies as a therapeutic or pharmaceutical preparation for human use.

E88. The method of embodiment E87, wherein the target a microorganism, a cancer, or a toxin.

E89. A method of treating a human subject in need thereof, comprising administering to the human subject a preparation of polyclonal antibodies produced by a non-human animal having a humanized immune system immunized, wherein the non-human animal having a humanized immune system has been immunized with the circular polyribonucleotide of any one of the preceding embodiments.

E90. The method of embodiment E89, wherein the human subject is at risk for exposure to a disease or condition associated with the antigen of the any one of the preceding embodiments or has been diagnosed with a disease associated with the antigen of any one of the preceding embodiments.

E91. The method of any one of embodiments E89 or E90, wherein the antigen is a pathogenic virus or bacteria and the subject is at risk or is diagnosed with a disease or condition caused by the pathogenic virus or bacteria.

E92. The method of any one of the preceding embodiments, wherein the human subject is at risk for or has been diagnosed with a cancer and the antigen is a cancer antigen.

E93. The method of any one of the preceding embodiments, wherein the human subject was bitten or stung by a venomous animal, ingested a toxin, or overdosed on a drug.

E94. The method of any one of the preceding embodiments, wherein the administration is before, after, or simultaneously with a human subject's risk of exposure to the disease.

E95. An antibody produced by administering the circular polyribonucleotide of any one of the preceding embodiments to a non-human animal having a humanized immune system.

E96. A plurality of polyclonal antibodies produced by immunizing a non-human animal having a humanized immune system with the circular polyribonucleotide of any one of the preceding embodiments.

E97. The antibody or plurality of polyclonal antibodies of any one of the preceding embodiments, further comprising a pharmaceutically acceptable carrier or excipient.

### Definitions

The present invention will be described with respect to particular embodiments and with reference to certain figures, but the invention is not limited thereto but only by the claims. Terms as set forth hereinafter are generally to be understood in their common sense unless indicated otherwise.

As used herein, the terms “circRNA” or “circular polyribonucleotide” or “circular RNA” are used interchangeably and mean a polyribonucleotide molecule that has a structure having no free ends (i.e., no free 3′ and/or 5′ ends), for example a polyribonucleotide that forms a circular or endless structure through covalent or non-covalent bonds.

As used herein, the terms “circRNA preparation” or “circular polyribonucleotide preparation” or “circular RNA preparation” are used interchangeably and mean a composition comprising circRNA molecules and a diluent, carrier, first adjuvant, or a combination thereof. An “immunogenic” circRNA preparation is that which when introduced into an animal causes the animal's immune system to become reactive against the antigen(s) expressed by the circRNA or a sequence of the circRNA.

As used herein, the term “total ribonucleotide molecules” means the total amount of any ribonucleotide molecules, including linear polyribonucleotide molecules, circular polyribonucleotide molecules, monomeric ribonucleotides, other polyribonucleotide molecules, fragments thereof, and modified variations thereof, as measured by total mass of the ribonucleotide molecules.

As used herein, the term “fragment” means any portion of a nucleotide molecule that is at least one nucleotide shorter than the nucleotide molecule. For example, a nucleotide molecule can be a linear polyribonucleotide molecule and a fragment thereof can be a monoribonucleotide or any number of contiguous polyribonucleotides that are a portion of the linear polyribonucleotide molecule. As another example, a nucleotide molecule can be a circular polyribonucleotide molecule and a fragment thereof can be a polyribonucleotide or any number of contiguous polyribonucleotides that are a portion of the circular polyribonucleotide molecule. A fragment of a nucleotide molecule includes at least 10 nucleic acid residues, e.g., at least 20 nucleic acid residues, at least 50 nucleic acid residues, and at least 100 nucleic acid residues. A “fragment” also means any portion of a polypeptide molecule that is at least one peptide shorter than the polypeptide molecule. For example, a fragment of a polypeptide can be a polypeptide or any number of contiguous amino acids that are a portion of the full-length polypeptide molecule. A fragment of a polypeptide includes at least 5 amino acid residues, e.g., at least 10 amino acids residues, at least 20 amino acids residues, at least 50 amino acid residues, at least 100 amino acid residues.

As used herein, the term “expression sequence” is a nucleic acid sequence that encodes a product, e.g., a peptide or polypeptide, or a regulatory nucleic acid. An exemplary expression sequence that codes for a peptide or polypeptide can comprise a plurality of nucleotide triads, each of which can code for an amino acid and is termed as a “codon”.

As used herein, the terms “linear RNA” or “linear polyribonucleotide” or “linear polyribonucleotide molecule” are used interchangeably and mean polyribonucleotide molecule having a 5′ and 3′ end. One or both of the 5′ and 3′ ends may be free ends or joined to another moiety. As used herein, a linear RNA has not undergone circularization (e.g., is pre-circularized) and can be used as a starting material for circularization through, for example, splint ligation, or chemical, enzymatic, ribozyme- or splicing-catalyzed circularization methods.

As used herein, the term “modified ribonucleotide” is a nucleotide with at least one modification to the sugar, the nucleobase, or the internucleoside linkage.

As used herein, the phrase “quasi-helical structure” is a higher order structure of the circular polyribonucleotide, wherein at least a portion of the circular polyribonucleotide folds into a helical structure.

As used herein, the phrase “quasi-double-stranded secondary structure” is a higher order structure of the circular polyribonucleotide, wherein at least a portion of the circular polyribonucleotide creates an internal double strand.

As used herein, the term “regulatory element” is a moiety, such as a nucleic acid sequence, that modifies expression of an expression sequence within the circular polyribonucleotide.

As used herein, the term “repetitive nucleotide sequence” is a repetitive nucleic acid sequence within a stretch of DNA or RNA or throughout a genome. In some embodiments, the repetitive nucleotide sequence includes poly CA or poly TG (UG) sequences. In some embodiments, the repetitive nucleotide sequence includes repeated sequences in the Alu family of introns.

As used herein, the term “replication element” is a sequence and/or motifs useful for replication or that initiate transcription of the circular polyribonucleotide.

As used herein, the term “stagger element” is a moiety, such as a nucleotide sequence, that induces ribosomal pausing during translation. In some embodiments, the stagger element is a non-conserved sequence of amino-acids with a strong alpha-helical propensity followed by the consensus sequence −D(V/I)ExNPG P, where x=any amino acid (SEQ ID NO: 22). In some embodiments, the stagger element may include a chemical moiety, such as glycerol, a non-nucleic acid linking moiety, a chemical modification, a modified nucleic acid, or any combination thereof.

As used herein, the term “substantially resistant” is one that has at least 50%, 55%, 60%, 65%, 70%, 75%, 80%, 85%, 90%, 95%, 96%, 97%, 98% or 99% resistance to an effector as compared to a reference.

As used herein, the term “stoichiometric translation” is a substantially equivalent production of expression products translated from the circular polyribonucleotide. For example, for a circular polyribonucleotide having two expression sequences, stoichiometric translation of the circular polyribonucleotide means that the expression products of the two expression sequences have substantially equivalent amounts, e.g., amount difference between the two expression sequences (e.g., molar difference) can be about 0, or less than 1%, 2%, 3%, 4%, 5%, 6%, 7%, 8%, 9%, 10%, 15%, or 20%, or any percentage therebetween.

As used herein, the term “translation initiation sequence” is a nucleic acid sequence that initiates translation of an expression sequence in the circular polyribonucleotide.

As used herein, the term “termination element” is a moiety, such as a nucleic acid sequence, that terminates translation of the expression sequence in the circular polyribonucleotide.

As used herein, the term “translation efficiency” is a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., an in vitro translation system like rabbit reticulocyte lysate, or an in vivo translation system like a eukaryotic cell or a prokaryotic cell.

As used herein, the term “circularization efficiency” is a measurement of resultant circular polyribonucleotide versus its non-circular starting material.

As used herein, the term “adaptive immune response” means either a humoral or cell-mediated immune response. The humoral immune response (also called antibody immune response) is mediated by B lymphocytes, which release antibodies that specifically bind to an antigen. The cell-mediated immune response (also called cellular immune response) involves the binding of cytotoxic T lymphocytes (CTL) to foreign or infected cells, followed by the lysis of these cells.

As used herein, the term “adjuvant” refers to a compound that, when used in combination with a circular RNA molecule, augments or otherwise alters or modifies the resultant immune response. Modification of the immune response includes intensification or broadening the specificity of either or both antibody and cellular immune responses. Modification of the immune response can also mean decreasing or suppressing certain antigen-specific immune responses.

As used herein, the terms “human antibody,” “human immunoglobulin,” or “human polyclonal antibodies” are used interchangeably and mean an antibody or antibodies produced in a non-human animal that is otherwise indistinguishable from antibody produced in a human vaccinated by the same circular RNA preparation. This is in contrast to “humanized antibodies” which are modified to have human characteristics, such as through generation of chimeras, but that maintain attributes of the host animal in which they are generated. Because human antibody made according to the method disclosed herein is comprised of IgG that are fully human, no enzymatic treatment is needed to eliminate the risk of anaphylaxis and serum sickness associated with heterologous species IgG.

As used herein, the term “linear counterpart” is a polyribonucleotide molecule (and its fragments) having the same or similar nucleotide sequence (e.g., 100%, 95%, 90%, 85%, 80%, 75%, or any percentage therebetween sequence similarity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence similarity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween sequence similarity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further comprises a 5′ cap. In some embodiments, the linear counterpart further comprises a poly adenosine tail. In some embodiments, the linear counterpart further comprises a 3′ UTR. In some embodiments, the linear counterpart further comprises a 5′ UTR.

As used herein, the term “carrier” means a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide, such as a lipid nanoparticle or LNP), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the term “naked”, “naked delivery” and its cognates means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that comprises a circular polyribonucleotide without covalent modification and is free from a carrier. A naked delivery formulation may comprise non-carrier pharmaceutical excipients, or diluents.

The term “diluent” means a vehicle comprising an inactive solvent in which a composition described herein (e.g., a composition comprising a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate, lactose, sucrose, cellulose, microcrystalline cellulose, kaolin, mannitol, sorbitol, inositol, sodium chloride, dry starch, cornstarch, or powdered sugar.

As used herein, a “variant” refers to a polypeptide which includes at least one alteration, e.g., a substitution, insertion, deletion, and/or fusion, at one or more residue positions, as compared to the parent or wild-type polypeptide. A variant may include between 1 and 10, 10 and 20, 20 and 50, 50 and 100, or more alterations.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic for producing human polyclonal antibodies that bind to an antigen (expressed from a circular polyribonucleotide) to be administered to human subjects.

FIG. 2 shows an RBD antigen encoded by a circular RNA was detected in BJ Fibroblasts and HeLa cells and was not detected in BJ Fibroblasts and HeLa cells with the vehicle control.

FIG. 3 shows that a sustainable anti-RBD antibody response was attained following administration of a circular RNA encoding a SARS-CoV-2 RBD antigen, formulated with a cationic polymer (e.g., protamine), in a mouse model.

FIG. 4 shows that an anti-Spike response was attained following administration of a circular RNA encoding a SARS-CoV-2 RBD antigen, formulated with a cationic polymer (e.g., protamine), in a mouse model.

FIG. 5 shows anti-RBD IgG2a and IgG1 isotype levels that were obtained after administration of a circular RNA encoding a SARS-CoV-2 RBD antigen, formulated with a cationic polymer (e.g., protamine), in a mouse model.

FIG. 6 shows protein expression from circular RNA in vivo for prolonged periods of time after intramuscular injection of circular RNA preparations (Trans-IT formulated, protamine formulated, unformulated), protamine vehicle only, and uninjected control mice.

FIG. 7 shows protein expression from circular RNA in vivo for prolonged periods of time after simultaneous intramuscular delivery of Addavax™ adjuvant with (i) unformulated circular RNA preparations (left graph), (ii) circular RNA formulated with TransIT (middle graph), and (iii) circular RNA formulated with protamine (right graph). In each case, Addavax™ adjuvant was delivered as an individual injection at 0 and 24 h.

FIG. 8 shows protein expression from circular RNA in vivo for prolonged periods of time after intradermal delivery of (i) circular RNA formulated with protamine, (ii) circular RNA formulated with protamine, with an injection of Addavax™ adjuvant at 24 hours, (iii) protamine vehicle only, and (iv) an uninjected control mice.

### DETAILED DESCRIPTION

The disclosure relates generally to compositions and methods for making and using polyclonal antibodies against a target, e.g., a pathogen, a cancer, or a toxin. The compositions, and the methods use, include a circular polyribonucleotide that includes a sequence that encodes an antigen (e.g., an epitope) of the target. The produced polyclonal antibodies can be used to treat a disease or condition (e.g., in a human subject) caused by the pathogen or the cancer expressing the antigen or associated with the toxin comprising the antigen.

The disclosure also relates to a method of administering a circular polyribonucleotide that comprises an antigenic sequence or encodes antigens and/or epitopes of a target, to a non-human animal with a humanized immune system, to stimulate production of human polyclonal antibodies that bind to the antigens and/or epitopes. In some embodiments, the circular polyribonucleotide comprises a sequence encoding an antigen from a microorganism target (e.g., a pathogenic microorganism), a cancer, or a toxin.

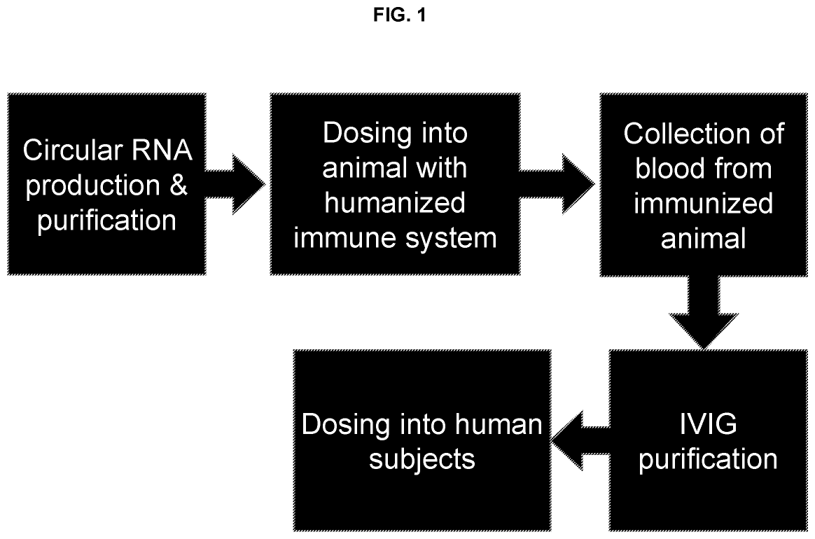

In some embodiments, the polyclonal antibodies produced bind to a target antigen and/or epitope expressed from a circular polyribonucleotide in a non-human animal having a humanized immune system. In further embodiments, the produced human polyclonal antibodies are purified. The purified human polyclonal antibodies are used to treat a disease or condition associated with the target. In some embodiments, the disease is caused by a microorganism, such as a virus (e.g., a coronavirus). In some embodiments, the disease is a cancer and the antigen is expressed by the cancer (e.g., is a neoantigen). In some embodiments, the condition is toxicity associated with a toxin and toxin comprises the antigen. In some embodiments, the produced human polyclonal antibodies are administered to a subject at risk of exposure to the disease caused by the target microorganism or at risk of developing the cancer expressing the antigen. In some embodiments, the produced human polyclonal antibodies are administered to a subject after being bitten or stung by a venomous animal, absorbing a toxin, inhaling a toxin, ingesting a toxin, or after overdosing on a drug. In certain embodiments, the produced polyclonal antibodies are used as an antivenom. A schematic example of the methods described herein is provided in FIG. 1.

### Compositions Comprising Circular Polyribonucleotides

Compositions of circular polyribonucleotides in a non-human animal having a humanized immune system are disclosed herein. Compositions of circular polyribonucleotides in a non-human animal having a humanized immune system are used for producing human polyclonal antibodies. In some embodiments, the circular polyribonucleotide comprises an antigenic sequence. In some embodiments, the antigen is from a microorganism, a cancer, or a toxin. In some embodiments, the produced human polyclonal antibodies are purified and used to treat a human subject in need thereof. In some embodiments, the circular polyribonucleotide comprises a sequence encoding a peptide, wherein the peptide expressed from the circular polyribonucleotide comprises the antigen. In some embodiments, a non-human animal having a humanized immune system is immunized with the circular polyribonucleotide or an immunogenic composition comprising the circular polyribonucleotide, to produce human polyclonal antibodies. An immunogenic composition of the disclosure may comprise a diluent, a carrier, an adjuvant, or a combination thereof In some embodiments, the immunogenic composition and adjuvant are separately co-administered to the non-human animal having a humanized immune system. In some embodiments, the immunogenic composition and adjuvant are administered to the non-human animal having a humanized immune system at different times. In some embodiments, the non-human animal having a humanized immune system is administered a second agent (e.g., a second vaccine) in combination with the circular polyribonucleotide. In some embodiments, the immunogenic composition and a vaccine are administered to the non-human animal having a humanized immune system at different times.

A composition of a circular polyribonucleotide is administered to a non-human animal having a humanized immune system to produce human polyclonal antibodies. The produced human polyclonal antibodies are fully human antibodies that do not comprise characteristics of the non-human animal. Therefore, these human polyclonal antibodies do not require any further processing to humanize them and no enzymatic treatment is needed to eliminate the risk of anaphylaxis or serum sickness associated with the heterologous species IgG. Furthermore, antigen-specific, high-titer human polyclonal antibodies are produced using this composition. Therefore, in some embodiments, these compositions are used to rapidly produce human polyclonal antibodies that bind to antigens from new diseases and are subsequently used to induce an immune response against the new disease in a human for protection from or treatment of the new disease.

### Methods of Making Compositions of Circular Polyribonucleotides in a Non-Human Animal Having a Humanized Immune System

The compositions of circular polyribonucleotides in a non-human animal having a humanized immune system are made by immunizing the non-human animal having the humanized immune system with the circular polyribonucleotides.

The compositions of circular polyribonucleotides in a non-human animal having a humanized immune system stimulate the production of human polyclonal antibodies by stimulating the adaptive immune response in the non-human animal having a humanized immune system. In some embodiments, the adaptive immune response of the non-human animal having a humanized immune system comprises a stimulation of humanized B lymphocytes to release human antibodies that specifically bind to the antigenic sequence of or antigen expressed by the circular polyribonucleotide. In some embodiments, the adaptive immune response of the non-human animal having a humanized immune system comprises stimulating cell-mediated immune responses. In further embodiments, an adjuvant is administered to the non-human animal having a humanized immune system.

Immunization comprises administering a composition (e.g., an immunogenic composition comprising a circular polyribonucleotide) to a non-human animal having a humanized immune system. In some embodiments, the non-human animal having a humanized immune system comprises a humanized immunoglobulin gene locus. In some embodiments, the immunogenic composition comprises circular polyribonucleotide and a diluent, a carrier, an adjuvant, or a combination thereof. Immunogenic compositions of the invention may also comprise one or more immunoregulatory agents. Preferably, one or more of the immunoregulatory agents include one or more adjuvants. The adjuvants may include a TH1 adjuvant and/or a TH2 adjuvant, further discussed below. In some embodiments, the immunogenic composition comprises a diluent free of any carrier and is used for naked delivery of the circular polyribonucleotide to a non-human animal with a humanized immune system. In other embodiments, an immunogenic composition comprises a circular polyribonucleotide described herein and a carrier, e.g., an LNP (lipid nanoparticle).

In certain embodiments, a non-human animal having a humanized immune system is further administered an adjuvant. The adjuvant enhances the innate immune response, which in turn, enhances the adaptive immune response for the production of human polyclonal antibodies in the non-human animal having a humanized immune system. An adjuvant can be any adjuvant as disclosed herein. In certain embodiments, the adjuvant is formulated with the circular polyribonucleotide as a part of the immunogenic composition. In certain embodiments, the adjuvant is formulated separately from the circular polyribonucleotide. The adjuvant is co-administered (e.g., administered simultaneously) or administered at a different time than the circular polyribonucleotide to the non-human animal having a humanized immune system. For example, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after the circular polyribonucleotide or an immunogenic composition comprising the circular polyribonucleotide. In some embodiments, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before the circular polyribonucleotide. For example, the adjuvant is administered 1, 2, 3, 4, 5, 6, 7, 14, 21, 28, 35, 42, 49, 56, 63, 70, 77, or 84 days, or any day therebetween, after the circular polyribonucleotide. In some embodiments, the adjuvant is administered 1, 2, 3, 4, 5, 6, 7, 14, 21, 28, 35, 42, 49, 56, 63, 70, 77, or 84 days, or any day therebetween, before the circular polyribonucleotide. The adjuvant is administered to the same anatomical location or different anatomical location as the circular polyribonucleotide.

In some embodiments, a non-human animal having a humanized immune system is further immunized with a vaccine that is not a circular polyribonucleotide. For example, the vaccine is a vaccine as disclosed herein. The vaccine is co-administered (e.g., administered simultaneously) or administered at a different time than the circular polyribonucleotide to the non-human animal having a humanized immune system. For example, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after the circular polyribonucleotide. In some embodiments, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before the circular polyribonucleotide. For example, the vaccine is administered 1, 2, 3, 4, 5, 6, 7, 14, 21, 28, 35, 42, 49, 56, 63, 70, 77, or 84 days, or any day therebetween, after the circular polyribonucleotide. In some embodiments, the vaccine is administered 1, 2, 3, 4, 5, 6, 7, 14, 21, 28, 35, 42, 49, 56, 63, 70, 77, or 84 days, or any day therebetween, before the circular polyribonucleotide.

A non-human animal having a humanized immune system is immunized with a circular polyribonucleotide (e.g., an immunogenic composition comprising a circular polyribonucleotide), adjuvant, vaccine (e.g., protein subunit vaccine), or a combination thereof any suitable number of times to achieve a desired response. For example, a prime-boost immunization strategy can be utilized to generate hyperimmune plasma containing a high concentration of antibodies that bind to antigens and/or epitopes of the disclosure. A non-human animal having a humanized immune system can be immunized with a circular polyribonucleotide, adjuvant, vaccine (e.g., protein subunit vaccine), or a combination thereof, of the disclosure, for example, at least 1, at least 2, at least 3, at least 4, at least 5, at least 6, at least 7, at least 8, at least 9, at least 10, or at least 15 times, or more.

In some embodiments, a non-human animal having a humanized immune system is immunized with a circular polyribonucleotide, adjuvant, vaccine (e.g., protein subunit vaccine), or a combination thereof, of the disclosure at most 2, at most 3, at most 4, at most 5, at most 6, at most 7, at most 8, at most 9, at most 10, at most 15, or at most 20 times, or less.

In some embodiments, a non-human animal having a humanized immune system is immunized with a circular polyribonucleotide, adjuvant, vaccine (e.g., protein subunit vaccine), or a combination thereof, of the disclosure about 1, 2, 3, 4, 5, 6, 7, 8, 9, 10, 15, or 20 times.

In some embodiments, a non-human animal having a humanized immune system is immunized with a circular polyribonucleotide (e.g., an immunogenic composition comprising the circular polyribonucleotide), adjuvant, vaccine (e.g., protein subunit vaccine), or a combination thereof, of the disclosure once. In some embodiments, a non-human animal having a humanized immune system is immunized with a circular polyribonucleotide (e.g., an immunogenic composition), adjuvant, vaccine (e.g., protein subunit vaccine), or a combination thereof, of the disclosure twice. In some embodiments, a non-human animal having a humanized immune system is immunized with a circular polyribonucleotide (e.g., an immunogenic composition), adjuvant, vaccine (e.g., protein subunit vaccine), or a combination thereof, of the disclosure three times. In some embodiments, a non-human animal having a humanized immune system is immunized with a circular polyribonucleotide (e.g., an immunogenic composition), adjuvant, vaccine (e.g., protein subunit vaccine), or a combination thereof, of the disclosure four times. In some embodiments, a non-human animal having a humanized immune system is immunized with a circular polyribonucleotide (e.g., an immunogenic composition), adjuvant, vaccine (e.g., protein subunit vaccine), or a combination thereof, of the disclosure five times. In some embodiments, a non-human animal having a humanized immune system is immunized with a circular polyribonucleotide (e.g., an immunogenic composition), adjuvant, vaccine (e.g., protein subunit vaccine), or a combination thereof, of the disclosure seven times.

Suitable time intervals are selected for spacing two or more immunizations. The time intervals apply to multiple immunizations with the same circular polyribonucleotide (e.g., immunogenic composition), adjuvant, or vaccine (e.g., protein subunit vaccine), or combination thereof, for example, the same circular polyribonucleotide (e.g., immunogenic composition), adjuvant, or vaccine (e.g., protein subunit vaccine), or combination thereof, is administered in the same amount or a different amount, via the same immunization route or a different immunization route. The time intervals apply to immunizations with different agents, for example, an immunogenic composition comprising a first circular polyribonucleotide and an immunogenic composition comprising a second circular polyribonucleotide. For regimens comprising three or more immunizations, the time intervals between immunizations are the same or different. In some examples, about 1, 2, 3, 4, 5, 6, 7, 8, 9, 10, 11, 12, 14, 16, 17, 18, 20, 22, 24, 26, 28, 30, 32, 34, 36, 40, 48, or 72 hours elapse between two immunizations. In some embodiments, about 1, 2, 3, 4, 5, 6, 7, 8, 9, 10, 11, 12, 14, 16, 17, 18, 20, 21, 24, 28, or 30 days elapse between two immunizations. In some embodiments, about 1, 2, 3, 4, 5, 6, 7, or 8 weeks elapse between two immunizations. In some embodiments, about 1, 2, 3, 4, 5, 6, 7, or 8 months elapse between two immunizations.

In some embodiments, at least 1, at least 2, at least 3, at least 4, at least 5, at least 6, at least 7, at least 8, at least 9, at least 10, at least 15, at least 20, at least 24, at least 36, or at least 72 hours, or more elapse between two immunizations. In some embodiments, at most 1, at most 2, at most 3, at most 4, at most 5, at most 6, at most 7, at most 8, at most 9, at most 10, at most 15, at most 20, at most 24, at most 36, or at most 72 hours, or less elapse between two immunizations.

In some embodiments, at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, elapse between two inoculations. In some embodiments, at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most 32, at most 34, or at most 36 days, or less elapse between two immunizations.

In some embodiments, at least 1, at least 2, at least 3, at least 4, at least 5, at least 6, at least 7, or at least 8 weeks, or more elapse between two immunizations. In some embodiments, at most 2, at most 3, at most 4, at most 5, at most 6, at most 7, at most 8 weeks, or less elapse between two immunizations.

In some embodiments, at least 1, at least 2, at least 3, at least 4, at least 5, at least 6, at least 7, or at least 8 months, or more elapse between two immunizations. In some embodiments, at most 2, at most 3, at most 4, at most 5, at most 6, at most 7, at most 8 months, or less elapse between two immunizations.

In some embodiments, a non-human animal having a humanized immune system is immunized 3 times at 3-4 week intervals.

A non-human animal having a humanized immune system is immunized with a circular polyribonucleotide (or immunogenic compositions comprising the circular polyribonucleotide), adjuvant, or vaccine (e.g., protein subunit vaccine), or combination thereof, at any suitable number anatomical sites. The same circular polyribonucleotide (or immunogenic composition thereof), adjuvant, vaccine (e.g., protein subunit vaccine), or a combination thereof can be administered to multiple anatomical sites. Different circular polyribonucleotides (or immunogenic compositions thereof), adjuvants, vaccine (e.g., protein subunit vaccine) or a combination thereof can be administered to different anatomical sites. Different circular polyribonucleotides (or immunogenic compositions thereof), adjuvants, vaccines (e.g., protein subunit vaccines) or a combination thereof can be administered to the same anatomical site, or any combination thereof. For example, a circular polyribonucleotide (or immunogenic composition thereof) can be administered to two different anatomical sites, and/or a circular polyribonucleotide (or immunogenic composition thereof) can be administered to one anatomical site, and an adjuvant can be administered to a different anatomical site.

Immunization with a circular polyribonucleotide (or an immunogenic composition thereof), adjuvant, or vaccine (e.g., protein subunit vaccine), or combination thereof is by any suitable administration route. In some embodiments, the immunization is by injection, infusion, by ophthalmic administration, or by intranasal administration. In some cases, administration can be via inhalation. Non-limiting examples of administration routes include oral, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intrasternal, intracerebral, intraocular, intralesional, intracerebroventricular, intracisternal, or intraparenchymal. Two or more immunizations are done by the same route or by different routes.

Immunization at any two or more anatomical routes are via the same route of administration (e.g., intramuscular) or by two or more routes of administration. In some embodiments, a circular polyribonucleotide (or an immunogenic composition thereof), adjuvant, or vaccine (e.g., protein subunit vaccine), or combination thereof, of the disclosure is administered to at least 1, at least 2, at least 3, at least 4, at least 5, or at least 6 anatomical sites of a non-human animal having a humanized immune system. In some embodiments, a circular polyribonucleotide (or an immunogenic composition thereof), adjuvant, or vaccine (e.g., protein subunit vaccine), or combination thereof, of the disclosure is administered to at most 2, at most 3, at most 4, at most 5, at most 6, at most 7, at most 8, at most 9, or at most 10 anatomical sites of the non-human animal, or less. In some embodiments, a circular polyribonucleotide (or an immunogenic composition thereof), adjuvant, or vaccine (e.g., protein subunit vaccine), or combination thereof is administered to 1, 2, 3, 4, 5, 6, 7, 8, 9, 10 anatomical sites of a non-human animal having a humanized immune system.

The non-human animal having a humanized immune system is immunized with any number of circular polyribonucleotides. The non-human animal having a humanized immune system is immunized with, for example, at least 1 circular polyribonucleotide. A non-human animal having a non-humanized immune system is immunized with, for example, at least 2, at least 3, at least 4, at least 5, at least 6, at least 7, at least 8, at least 9, at least 10, at least 11, at least 12, at least 13, at least 14, at least 15, at least 20 different circular polyribonucleotides, or more different circular polyribonucleotides. In some embodiments, a non-human animal having a humanized immune system is immunized with at most 1 circular polyribonucleotide. In some embodiments, a non-human animal having a humanized immune system is immunized with at most 2, at most 3, at most 4, at most 5, at most 6, at most 7, at most 8, at most 9, at most 10, at most 11, at most 12, at most 13, at most 14, at most 15, at most 20 different circular polyribonucleotides, or less than 21 different circular polyribonucleotides. In some embodiments, a non-human animal having a humanized immune system is immunized with about 1 circular polyribonucleotide. In some embodiments, a non-human animal having a humanized immune system is immunized with about 2, about 3, about 4, about 5, about 6, about 7, about 8, about 9, about 10, about 11, about 12, about 13, about 14, about 15, or about 20 different circular polyribonucleotides. In some embodiments, a non-human animal having a humanized immune system is immunized with about 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 4-4, 4-3, 5-20, 5-15, 5-10, 5-9, 5-8, 5-7, 5-6, 5-10, 10-15, or 15-20 different circular polyribonucleotides. Different circular polyribonucleotides have different sequences from each other. For example, they can comprise or encode different antigens and/or epitopes, overlapping antigens and/or epitopes, similar antigens and/or epitopes, or the same antigens and/or epitopes (for example, with the same or different regulatory elements, initiation sequences, promoters, termination elements, or other elements of the disclosure). In cases where a non-human animal having a humanized immune system is immunized with two or more different circular polyribonucleotides, the two or more different circular polyribonucleotides are administered at the same time or at different times. The two or more different circular polyribonucleotides are administered to the same anatomical location or different anatomical locations.

Any suitable amount of a circular polyribonucleotide is administered to a non-human animal having a humanized immune system. In a particular embodiment, a non-human animal having a humanized immune system is immunized with at least about 1 ng, at least about 10 ng, at least about 100 ng, at least about 1 μg, at least about 10 μg, at least about, at least about 100 μg, at least about 1 mg, at least about 10 mg, at least about 100 mg, or at least about 1 g of a circular polyribonucleotide. In some embodiments, a non-human animal having a humanized immune system is immunized with at most about 1 ng, at most about 10 ng, at most about 100 ng, at most about 1 μg, at most about 10 μg, at most about, at most about 100 μg, at most about 1 mg, at most about 10 mg, at most about 100 mg, or at most about 1 g of a circular polyribonucleotide. In some embodiments, a non-human animal having a humanized immune system is immunized with about 1 ng, about 10 ng, about 100 ng, about 1 μg, about 10 μg, about, about 100 μg, about 1 mg, about 10 mg, about 100 mg, or about 1 g of a circular polyribonucleotide.

In some embodiments, the non-human animal having a humanized immune system or a cell or tissue therefrom is contacted with the circular polyribonucleotide.

The invention also includes: a non-human animal having a humanized immune system and comprising a circular polyribonucleotide comprising a sequence encoding an antigen; a non-human animal having a humanized immune system and comprising a circular polyribonucleotide comprising an antigenic sequence; a non-human mammal comprising a humanized immunoglobulin gene locus and a circular polyribonucleotide comprising a sequence encoding an antigen; a non-human mammal comprising a humanized immunoglobulin gene locus and a circular polyribonucleotide comprising an antigenic sequence. An antigenic sequence of a circRNA is that which when introduced into an animal causes the animal's immune system to become reactive against the polyribonucleotides comprising the sequence that is the antigenic sequence.

The amount of the circular polyribonucleotide, expression product, or both in the non-human animal having a humanized immune system or cell or tissue therefrom can be measured at any time after administration. In certain embodiments, a time course of an antibody response in the non-human animal having a humanized immune system is determined, for example, by a titer of total antibodies, antibodies of a given isotype, and/or or antibodies specific for antigens and/or epitopes of the disclosure. If the antibody titer is increased in the presence of the circular polyribonucleotide, the circular polyribonucleotide or expression product or both is identified as being effective in increasing the production of antibodies by the non-human animal having a humanized immune system.

### Components of the Compositions of Circular Polyribonucleotides in a Non-Human Animal Having a Humanized Immune System

Compositions as disclosed herein and used in the methods described herein comprise a circular polyribonucleotide comprising an antigenic sequence, a sequence encoding an antigen, or a combination thereof. In some embodiments, the circular polyribonucleotide is in an immunogenic composition. An immunogenic composition comprises (a) the circular polyribonucleotide, and (b) a diluent, carrier, and adjuvant, or any combination thereof. In some embodiments, an adjuvant is co-administered with or separately administered from the circular polyribonucleotide to the non-human animal having a humanized immune system. In some embodiments, a vaccine is co-administered with or separately administered from the circular polyribonucleotide to the non-human animal having a humanized immune system.

### Circular Polyribonucleotide

A circular polyribonucleotide is used to produce human polyclonal antibodies in a non-human animal having a humanized immune system. The circular polyribonucleotide comprises the elements as described below. Furthermore, the circular polyribonucleotide, in some embodiments, is immunized in a circular polyribonucleotide formulation comprising a diluent, carrier, adjuvant, or any combination thereof In some embodiments, formulation of the circular polyribonucleotide with a diluent free of any carrier is used for naked delivery of an immunization of the circular polyribonucleotide to the non-human animal having the humanized immune system.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the circular polyribonucleotide may be of a sufficient size to accommodate a binding site for a ribosome. In some embodiments, the maximum size of a circular polyribonucleotide can be as large as is within the technical constraints of producing a circular polyribonucleotide, and/or using the circular polyribonucleotide. Without wishing to be bound by any particular theory, it is possible that multiple segments of RNA may be produced from DNA and their 5′ and 3′ free ends annealed to produce a “string” of RNA, which ultimately may be circularized when only one 5′ and one 3′ free end remains. In some embodiments, the maximum size of a circular polyribonucleotide may be limited by the ability of packaging and delivering the RNA to a target. In some embodiments, the size of a circular polyribonucleotide is a length sufficient to encode useful polypeptides, such as antigens and/or epitopes of the disclosure, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides, or at least 70 nucleotides, may be useful.

### Circular Polyribonucleotide Elements

In some embodiments, the circular polyribonucleotide comprises one or more of the elements as described herein in addition to comprising an antigenic sequence or a sequence encoding an antigen and/or epitope. In some embodiments, the circular polyribonucleotide comprises any feature or any combination of features as disclosed in WO2019/118919, which is hereby incorporated by reference in its entirety. For example, the circular polyribonucleotide comprises a regulatory element, e.g., a sequence that modifies expression of an expression sequence within the circular polyribonucleotide. A regulatory element may include a sequence that is located adjacent to an expression sequence that encodes an expression product. A regulatory element may be operably linked to the adjacent sequence. A regulatory element may increase an amount of product expressed as compared to an amount of the expressed product when no regulatory element is present. In addition, one regulatory element can increase a number of products expressed for multiple expression sequences attached in tandem. Hence, one regulatory element can enhance the expression of one or more expression sequences. Multiple regulatory elements can also be used, for example, to differentially regulate expression of different expression sequences. In some embodiments, a regulatory element as provided herein can include a selective translation sequence. As used herein, the term “selective translation sequence” refers to a nucleic acid sequence that selectively initiates or activates translation of an expression sequence in the circular polyribonucleotide, for instance, certain riboswitch aptazymes. A regulatory element can also include a selective degradation sequence. As used herein, the term “selective degradation sequence” refers to a nucleic acid sequence that initiates degradation of the circular polyribonucleotide, or an expression product of the circular polyribonucleotide. In some embodiments, the regulatory element is a translation modulator. A translation modulator can modulate translation of the expression sequence in the circular polyribonucleotide. A translation modulator can be a translation enhancer or suppressor. In some embodiments, a translation initiation sequence can function as a regulatory element. Further examples of regulatory elements are described in paragraphs [0154]-[0161] of WO2019/118919, which is hereby incorporated by reference in its entirety.

In some embodiments, the circular polyribonucleotide encodes an antigen that produces the human polyclonal antibodies of interest and comprises a translation initiation sequence, e.g., a start codon. In some embodiments, the translation initiation sequence includes a Kozak or Shine-Dalgarno sequence. In some embodiments, the circular polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular polyribonucleotide includes at least one translation initiation sequence adjacent to an expression sequence. In some embodiments, the translation initiation sequence provides conformational flexibility to the circular polyribonucleotide. In some embodiments, the translation initiation sequence is within a substantially single stranded region of the circular polyribonucleotide. Further examples of translation initiation sequences are described in paragraphs [0163]-[0165] of WO2019/118919, which is hereby incorporated by reference in its entirety.

In some embodiments, a circular polyribonucleotide described herein comprises an internal ribosome entry site (IRES) element. A suitable IRES element to include in a circular polyribonucleotide can be an RNA sequence capable of engaging a eukaryotic ribosome. Further examples of an IRES are described in paragraphs [0166]-[0168] of WO2019/118919, which is hereby incorporated by reference in its entirety.

A circular polyribonucleotide can include one or more expression sequences (e.g., encoding an antigen), and each expression sequence may or may not have a termination element. Further examples of termination elements are described in paragraphs [0169]-[0170] of WO2019/118919, which is hereby incorporated by reference in its entirety.

A circular polyribonucleotide of the disclosure can comprise a stagger element. The term “stagger element” refers to a moiety, such as a nucleotide sequence, that induces ribosomal pausing during translation. In some embodiments, the stagger element is a non-conserved sequence of amino-acids with a strong alpha-helical propensity followed by the consensus sequence −D(V/I)ExNPGP, where x=any amino acid (SEQ ID NO: 22). In some embodiments, the stagger element may include a chemical moiety, such as glycerol, a non-nucleic acid linking moiety, a chemical modification, a modified nucleic acid, or any combination thereof.

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and/or polypeptide(s). In some embodiments, the stagger element is a portion of the one or more expression sequences. In some embodiments, the circular polyribonucleotide comprises one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element comprises a portion of an expression sequence of the one or more expression sequences.

Examples of stagger elements are described in paragraphs [0172]-[0175] of WO2019/118919, which is hereby incorporated by reference in its entirety.

In some embodiments, the circular polyribonucleotide comprises one or more regulatory nucleic acid sequences or comprises one or more expression sequences that encode regulatory nucleic acid, e.g., a nucleic acid that modifies expression of an endogenous gene and/or an exogenous gene. In some embodiments, the expression sequence of a circular polyribonucleotide as provided herein can comprise a sequence that is antisense to a regulatory nucleic acid like a non-coding RNA, such as, but not limited to, tRNA, lncRNA, miRNA, rRNA, snRNA, microRNA, siRNA, piRNA, snoRNA, snRNA, exRNA, scaRNA, Y RNA, and hnRNA.

Exemplary regulatory nucleic acids are described in paragraphs [0177]-[0194] of WO2019/118919, which is hereby incorporated by reference in its entirety.

In some embodiments, the translation efficiency of a circular polyribonucleotide as provided herein is greater than a reference, e.g., a linear counterpart, a linear expression sequence, or a linear circular polyribonucleotide. In some embodiments, a circular polyribonucleotide as provided herein has the translation efficiency that is at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 450%, 500%, 600%, 70%, 800%, 900%, 1000%, 2000%, 5000%, 10000%, 100000%, or more greater than that of a reference. In some embodiments, a circular polyribonucleotide has a translation efficiency 10% greater than that of a linear counterpart. In some embodiments, a circular polyribonucleotide has a translation efficiency 300% greater than that of a linear counterpart.

In some embodiments, the circular polyribonucleotide produces stoichiometric ratios of expression products. Rolling circle translation continuously produces expression products at substantially equivalent ratios. In some embodiments, the circular polyribonucleotide has a stoichiometric translation efficiency, such that expression products are produced at substantially equivalent ratios. In some embodiments, the circular polyribonucleotide has a stoichiometric translation efficiency of multiple expression products, e.g., products from 2, 3, 4, 5, 6, 7, 8, 9, 10, 11, 12, or more expression sequences.

In some embodiments, once translation of the circular polyribonucleotide is initiated, the ribosome bound to the circular polyribonucleotide does not disengage from the circular polyribonucleotide before finishing at least one round of translation of the circular polyribonucleotide. In some embodiments, the circular polyribonucleotide as described herein is competent for rolling circle translation. In some embodiments, during rolling circle translation, once translation of the circular polyribonucleotide is initiated, the ribosome bound to the circular polyribonucleotide does not disengage from the circular polyribonucleotide before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 105 rounds, or at least 106 rounds of translation of the circular polyribonucleotide.

In some embodiments, the rolling circle translation of the circular polyribonucleotide leads to generation of polypeptide product that is translated from more than one round of translation of the circular polyribonucleotide (“continuous” expression product). In some embodiments, the circular polyribonucleotide comprises a stagger element, and rolling circle translation of the circular polyribonucleotide leads to generation of polypeptide product that is generated from a single round of translation or less than a single round of translation of the circular polyribonucleotide (“discrete” expression product). In some embodiments, the circular polyribonucleotide is configured such that at least 10%, 20%, 30%, 40%, 50%, at least 60%, at least 70%, at least 80%, at least 90%, at least 95%, at least 96%, at least 97%, at least 98%, at least 99%, or 100% of total polypeptides (molar/molar) generated during the rolling circle translation of the circular polyribonucleotide are discrete polypeptides. In some embodiments, the amount ratio of the discrete products over the total polypeptides is tested in an in vitro translation system. In some embodiments, the in vitro translation system used for the test of amount ratio comprises rabbit reticulocyte lysate. In some embodiments, the amount ratio is tested in an in vivo translation system, such as a eukaryotic cell or a prokaryotic cell, a cultured cell or a cell in an organism.

In some embodiments, the circular polyribonucleotide comprises untranslated regions (UTRs). UTRs of a genomic region comprising a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for first expression sequence is the same as or continuous with or overlapping with another UTR for a second expression sequence. In some embodiments, the intron is a human intron. In some embodiments, the intron is a full length human intron, e.g., ZKSCAN1.

Exemplary untranslated regions are described in paragraphs [0197]-[201] of WO2019/118919, which is hereby incorporated by reference in its entirety.

In some embodiments, the circular polyribonucleotide may include a poly-A sequence. Exemplary poly-A sequences are described in paragraphs [0202]-[0205] of WO2019/118919, which is hereby incorporated by reference in its entirety.

In some embodiments, the circular polyribonucleotide comprises one or more riboswitches. Exemplary riboswitches are described in paragraphs [0232]-[0252] of WO2019/118919, which is hereby incorporated by reference in its entirety.

In some embodiments, the circular polyribonucleotide comprises an aptazyme. Exemplary aptazymes are described in paragraphs [0253]-[0259] of WO2019/118919, which is hereby incorporated by reference in its entirety.

In some embodiments, the circular polyribonucleotide comprises one or more RNA binding sites. microRNAs (or miRNA) can be short noncoding RNAs that bind to the 3′UTR of nucleic acid molecules and down-regulate gene expression either by reducing nucleic acid molecule stability or by inhibiting translation. The circular polyribonucleotide may comprise one or more microRNA target sequences, microRNA sequences, or microRNA seeds. Such sequences may correspond to any known microRNA, such as those taught in US Publication US2005/0261218 and US Publication US2005/0059005, the contents of which are incorporated herein by reference in their entirety. Further examples of RNA binding sites are described in paragraphs [0206]-[0215] of WO2019/118919, which is hereby incorporated by reference in its entirety.

In some embodiments, the circular polyribonucleotide includes one or more protein binding sites that enable a protein, e.g., a ribosome, to bind to an internal site in the RNA sequence. Further examples of protein binding sites are described in paragraphs [0218]-[0221] of WO2019/118919, which is hereby incorporated by reference in its entirety.

In some embodiments, the circular polyribonucleotide comprises a spacer sequence. In some embodiments, elements of a polyribonucleotide may be separated from one another by a spacer sequence or linker. Exemplary of spacer sequences are described in paragraphs [0293]-[0302] of WO2019/118919, which is hereby incorporated by reference in its entirety.

The circular polyribonucleotide described herein may also comprise a non-nucleic acid linker. Exemplary non-nucleic acid linkers are described in paragraphs [0303]-[0307] of WO2019/118919, which is hereby incorporated by reference in its entirety.

In some embodiments, the circular polyribonucleotide further includes another nucleic acid sequence. In some embodiments, the circular polyribonucleotide may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In some embodiments, the circular polyribonucleotide includes an siRNA to target a different locus of the same gene expression product as the circular polyribonucleotide. In some embodiments, the circular polyribonucleotide includes an siRNA to target a different gene expression product than a gene expression product that is present in the circular polyribonucleotide.

In some embodiments, the circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

In some embodiments, the circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, the circular polyribonucleotide lacks a poly-A sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide comprises one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

As a result of its circularization, the circular polyribonucleotide may include certain characteristics that distinguish it from linear RNA. For example, the circular polyribonucleotide is less susceptible to degradation by exonuclease as compared to linear RNA. As such, the circular polyribonucleotide can be more stable than a linear RNA, especially when incubated in the presence of an exonuclease. The increased stability of the circular polyribonucleotide compared with linear RNA can make the circular polyribonucleotide more useful as a cell transforming reagent to produce polypeptides (e.g., antigens and/or epitopes to elicit antibody responses). The increased stability of the circular polyribonucleotide compared with linear RNA can make the circular polyribonucleotide easier to store for long than linear RNA. The stability of the circular polyribonucleotide treated with exonuclease can be tested using methods standard in art which determine whether RNA degradation has occurred (e.g., by gel electrophoresis).

Moreover, unlike linear RNA, the circular polyribonucleotide can be less susceptible to dephosphorylation when the circular polyribonucleotide is incubated with phosphatase, such as calf intestine phosphatase.

In some embodiments, the circular polyribonucleotide comprises particular sequence characteristics. For example, the circular polyribonucleotide may comprise a particular nucleotide composition. In some such embodiments, the circular polyribonucleotide may include one or more purine (adenine and/or guanosine) rich regions. In some such embodiments, the circular polyribonucleotide may include one or more purine poor regions. In some embodiments, the circular polyribonucleotide may include one or more AU rich regions or elements (AREs). In some embodiments, the circular polyribonucleotide may include one or more adenine rich regions.

In some embodiments, the circular polyribonucleotide may include one or more repetitive elements described elsewhere herein. In some embodiments, the circular polyribonucleotide comprises one or more modifications described elsewhere herein.

A circular polyribonucleotide may include one or more substitutions, insertions and/or additions, deletions, and covalent modifications with respect to reference sequences. For example, circular polyribonucleotides with one or more insertions, additions, deletions, and/or covalent modifications relative to a parent polyribonucleotide are included within the scope of this disclosure. Exemplary modifications are described in paragraphs [0310]-[0325] of WO2019/118919, which is hereby incorporated by reference in its entirety.

In some embodiments, the circular polyribonucleotide comprises a higher order structure, e.g., a secondary or tertiary structure. In some embodiments, complementary segments of the circular polyribonucleotide fold itself into a double stranded segment, held together with hydrogen bonds between pairs, e.g., A-U and C-G. In some embodiments, helices, also known as stems, are formed intra-molecularly, having a double-stranded segment connected to an end loop. In some embodiments, the circular polyribonucleotide has at least one segment with a quasi-double-stranded secondary structure.

In some embodiments, one or more sequences of the circular polyribonucleotide include substantially single stranded vs double stranded regions. In some embodiments, the ratio of single stranded to double stranded may influence the functionality of the circular polyribonucleotide.

In some embodiments, one or more sequences of the circular polyribonucleotide that are substantially single stranded. In some embodiments, one or more sequences of the circular polyribonucleotide that are substantially single stranded may include a protein- or RNA-binding site. In some embodiments, the circular polyribonucleotide sequences that are substantially single stranded may be conformationally flexible to allow for increased interactions. In some embodiments, the sequence of the circular polyribonucleotide is purposefully engineered to include such secondary structures to bind or increase protein or nucleic acid binding.

In some embodiments, the circular polyribonucleotide sequences that are substantially double stranded. In some embodiments, one or more sequences of the circular polyribonucleotide that are substantially double stranded may include a conformational recognition site, e.g., a riboswitch or aptazyme. In some embodiments, the circular polyribonucleotide sequences that are substantially double stranded may be conformationally rigid. In some such instances, the conformationally rigid sequence may sterically hinder the circular polyribonucleotide from binding a protein or a nucleic acid. In some embodiments, the sequence of the circular polyribonucleotide is purposefully engineered to include such secondary structures to avoid or reduce protein or nucleic acid binding.

There are 16 possible base-pairings, however of these, six (AU, GU, GC, UA, UG, CG) may form actual base-pairs. The rest are called mismatches and occur at very low frequencies in helices. In some embodiments, the structure of the circular polyribonucleotide cannot easily be disrupted without impact on its function and lethal consequences, which provide a selection to maintain the secondary structure. In some embodiments, the primary structure of the stems (i.e., their nucleotide sequence) can still vary, while still maintaining helical regions. The nature of the bases is secondary to the higher structure, and substitutions are possible as long as they preserve the secondary structure. In some embodiments, the circular polyribonucleotide has a quasi-helical structure. In some embodiments, the circular polyribonucleotide has at least one segment with a quasi-helical structure. In some embodiments, the circular polyribonucleotide includes at least one of a U-rich or A-rich sequence or a combination thereof. In some embodiments, the U-rich and/or A-rich sequences are arranged in a manner that would produce a triple quasi-helix structure. In some embodiments, the circular polyribonucleotide has a double quasi-helical structure. In some embodiments, the circular polyribonucleotide has one or more segments (e.g., 2, 3, 4, 5, 6, or more) having a double quasi-helical structure. In some embodiments, the circular polyribonucleotide includes at least one of a C-rich and/or G-rich sequence. In some embodiments, the C-rich and/or G-rich sequences are arranged in a manner that would produce triple quasi-helix structure. In some embodiments, the circular polyribonucleotide has an intramolecular triple quasi-helix structure that aids in stabilization.

In some embodiments, the circular polyribonucleotide has two quasi-helical structure (e.g., separated by a phosphodiester linkage), such that their terminal base pairs stack, and the quasi-helical structures become colinear, resulting in a “coaxially stacked” substructure.

In some embodiments, the circular polyribonucleotide comprises a tertiary structure with one or more motifs, e.g., a pseudoknot, a g-quadruplex, a helix, and coaxial stacking.

Further examples of structure of circular polyribonucleotides as disclosed herein are described in paragraphs [0326]-[0333] of WO2019/118919, which is hereby incorporated by reference in its entirety.

### Stability and Half Life

In some embodiments, a circular polyribonucleotide provided herein has increased half-life over a reference, e.g., a linear polyribonucleotide having the same nucleotide sequence that is not circularized (linear counterpart). In some embodiments, the circular polyribonucleotide is substantially resistant to degradation, e.g., exonuclease degradation. In some embodiments, the circular polyribonucleotide is resistant to self-degradation. In some embodiments, the circular polyribonucleotide lacks an enzymatic cleavage site, e.g., a dicer cleavage site. Further examples of stability and half life of circular polyribonucleotides as disclosed herein are described in paragraphs [0308]-[0309] of WO2019/118919, which is hereby incorporated by reference in its entirety.

### Production Methods

In some embodiments, the circular polyribonucleotide includes a deoxyribonucleic acid sequence that is non-naturally occurring and can be produced using recombinant technology (e.g., derived in vitro using a DNA plasmid), chemical synthesis, or a combination thereof.

It is within the scope of the disclosure that a DNA molecule used to produce an RNA circle can comprise a DNA sequence of a naturally-occurring original nucleic acid sequence, a modified version thereof, or a DNA sequence encoding a synthetic polypeptide not normally found in nature (e.g., chimeric molecules or fusion proteins, such as fusion proteins comprising multiple antigens and/or epitopes). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

The circular polyribonucleotide may be prepared according to any available technique including, but not limited to chemical synthesis and enzymatic synthesis. In some embodiments, a linear primary construct or linear mRNA may be cyclized, or concatemerized to create a circular polyribonucleotide described herein. The mechanism of cyclization or concatemerization may occur through methods such as, but not limited to, chemical, enzymatic, splint ligation), or ribozyme catalyzed methods. The newly formed 5′-/3′-linkage may be an intramolecular linkage or an intermolecular linkage.

Methods of making the circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Various methods of synthesizing circular polyribonucleotides are also described in the art (see, e.g., U.S. Pat. Nos. 6,210,931, 5,773,244, 5,766,903, 5,712,128, 5,426,180, US Publication No. US20100137407, International Publication No. WO1992001813 and International Publication No. WO2010084371; the contents of each of which are herein incorporated by reference in their entireties).

In some embodiments, the circular polyribonucleotides may be cleaned up after production to remove production impurities, e.g., free ribonucleic acids, linear or nicked RNA, DNA, proteins, etc. In some embodiments, the circular polyribonucleotides may be purified by any known method commonly used in the art. Examples of nonlimiting purification methods include, column chromatography, gel excision, size exclusion, etc.

### Circularization

In some embodiments, a linear circular polyribonucleotide may be cyclized, or concatemerized. In some embodiments, the linear circular polyribonucleotide may be cyclized in vitro prior to formulation and/or delivery. In some embodiments, the linear circular polyribonucleotide may be cyclized within a cell.

### Extracellular Circularization

In some embodiments, the linear circular polyribonucleotide is cyclized, or concatemerized using a chemical method to form a circular polyribonucleotide. In some chemical methods, the 5′-end and the 3′-end of the nucleic acid (e.g., a linear circular polyribonucleotide) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In some embodiments, a DNA or RNA ligase is used to enzymatically link a 5′-phosphorylated nucleic acid molecule (e.g., a linear circular polyribonucleotide) to the 3′-hydroxyl group of a nucleic acid (e.g., a linear nucleic acid) forming a new phosphorodiester linkage. In an example reaction, a linear circular polyribonucleotide is incubated at 37° C. for 1 hour with 1-10 units of T4 RNA ligase (New England Biolabs, Ipswich, Mass.) according to the manufacturer's protocol. The ligation reaction may occur in the presence of a linear nucleic acid capable of base-pairing with both the 5′- and 3′-region in juxtaposition to assist the enzymatic ligation reaction. In some embodiments, the ligation is splint ligation. For example, a splint ligase, like SplintR® ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase is used in the synthesis of the circular polynucleotides. As a non-limiting example, the ligase may be a circ ligase or circular ligase.

In some embodiments, either the 5′-or 3′-end of the linear circular polyribonucleotide can encode a ligase ribozyme sequence such that during in vitro transcription, the resultant linear circular polyribonucleotide includes an active ribozyme sequence capable of ligating the 5′-end of the linear circular polyribonucleotide to the 3′-end of the linear circular polyribonucleotide. The ligase ribozyme may be derived from the Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, a linear circular polyribonucleotide is cyclized or concatermerized by using at least one non-nucleic acid moiety. In one aspect, the at least one non-nucleic acid moiety may react with regions or features near the 5′ terminus and/or near the 3′ terminus of the linear circular polyribonucleotide in order to cyclize or concatermerize the linear circular polyribonucleotide. In another aspect, the at least one non-nucleic acid moiety may be located in or linked to or near the 5′ terminus and/or the 3′ terminus of the linear circular polyribonucleotide. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In some embodiments, a linear circular polyribonucleotide is cyclized or concatermerized due to a non-nucleic acid moiety that causes an attraction between atoms, molecular surfaces at, near or linked to the 5′ and 3′ ends of the linear circular polyribonucleotide. As a non-limiting example, one or more linear circular polyribonucleotides may be cyclized or concatermized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In some embodiments, the linear circular polyribonucleotide may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′terminus may associate with each other causing a linear circular polyribonucleotide to cyclize or concatemerize. In another aspect, the peptides covalently linked to the ribozyme RNA near the 5′ terminus and the 3′ terminus may cause the linear primary construct or linear mRNA to cyclize or concatemerize after being subjected to ligated using various methods known in the art such as, but not limited to, protein ligation. Non-limiting examples of ribozymes for use in the linear primary constructs or linear RNA of the present invention or a non-exhaustive listing of methods to incorporate and/or covalently link peptides are described in US patent application No. US20030082768, the contents of which is here in incorporated by reference in its entirety.

In some embodiments, the linear circular polyribonucleotide may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear circular polyribonucleotide into a 5′ monophosphate may occur by a two-step reaction comprising: (a) contacting the 5′ nucleotide of the linear circular polyribonucleotide with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%. In some embodiments, the circularization method provided has a circularization efficiency of between about 10% and about 100%; for example, the circularization efficiency may be about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In some embodiments, the circularization efficiency is between about 20% and about 80%. In some embodiments, the circularization efficiency is between about 30% and about 60%. In some embodiments the circularization efficiency is about 40%.

### Splicing Element

In some embodiment, the circular polyribonucleotide includes at least one splicing element. Exemplary splicing elements are described in paragraphs [0270]-[0275] of WO2019/118919, which is hereby incorporated by reference in its entirety.

In some embodiments, a circular polyribonucleotide includes at least one splicing element. In a circular polyribonucleotide as provided herein, a splicing element can be a complete splicing element that can mediate splicing of the circular polyribonucleotide. Alternatively, the splicing element can also be a residual splicing element from a completed splicing event. For instance, in some cases, a splicing element of a linear polyribonucleotide can mediate a splicing event that results in circularization of the linear polyribonucleotide, thereby the resultant circular polyribonucleotide includes a residual splicing element from such splicing-mediated circularization event. In some cases, the residual splicing element is not able to mediate any splicing. In other cases, the residual splicing element can still mediate splicing under certain circumstances. In some embodiments, the splicing element is adjacent to at least one expression sequence. In some embodiments, the circular polyribonucleotide includes a splicing element adjacent each expression sequence. In some embodiments, the splicing element is on one or both sides of each expression sequence, leading to separation of the expression products, e.g., peptide(s) and or polypeptide(s).

In some embodiments, a circular polyribonucleotide includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, Alu elements in flanking introns, and motifs found in (suptable4 enriched motifs) cis-sequence elements proximal to backsplice events such as sequences in the 200 bp preceding (upstream of) or following (downstream from) a backsplice site with flanking exons. In some embodiments, the circular polyribonucleotide includes at least one repetitive nucleotide sequence described elsewhere herein as an internal splicing element. In such embodiments, the repetitive nucleotide sequence may include repeated sequences from the Alu family of introns. In some embodiments, a splicing-related ribosome binding protein can regulate circular polyribonucleotide biogenesis (e.g. the Muscleblind and Quaking (QKI) splicing factors).

In some embodiments, a circular polyribonucleotide may include canonical splice sites that flank head-to-tail junctions of the circular polyribonucleotide.

In some embodiments, a circular polyribonucleotide may include a bulge-helix-bulge motif, including a 4-base pair stem flanked by two 3-nucleotide bulges. Cleavage occurs at a site in the bulge region, generating characteristic fragments with terminal 5′-hydroxyl group and 2′,3′-cyclic phosphate. Circularization proceeds by nucleophilic attack of the 5′-OH group onto the 2′,3′-cyclic phosphate of the same molecule forming a 3′,5′-phosphodiester bridge.

In some embodiments, a circular polyribonucleotide may include a multimeric repeating RNA sequence that harbors a HPR element. The HPR includes a 2′,3′-cyclic phosphate and a 5′-OH termini. The HPR element self-processes the 5′- and 3′-ends of the linear polyribonucleotide for circularization, thereby ligating the ends together.

In some embodiments, a circular polyribonucleotide may include a self-splicing element. For example, the circular polyribonucleotide may include an intron from the cyanobacteria Anabaena.

In some embodiments, a circular polyribonucleotide may include a sequence that mediates self-ligation. In one embodiment, the circular polyribonucleotide may include a HDV sequence (e.g., HDV replication domain conserved sequence, GGCUCAUCUCGACAAGAGGCGGCAGUCCUCAGUACUCUUACUCUUUUCUGUAAAGAGGAG ACUGCUGGACUCGCCGCCCAAGUUCGAGCAUGAGCC (SEQ ID NO: 25) or GGCUAGAGGCGGCAGUCCUCAGUACUCUUACUCUUUUCUGUAAAGAGGAGACUGCUGGA CUCGCCGCCCGAGCC (SEQ ID NO: 26)) to self-ligate. In one embodiment, the circular polyribonucleotide may include loop E sequence (e.g., in PSTVd) to self-ligate. In another embodiment, the circular polyribonucleotide may include a self-circularizing intron, e.g., a 5′ and 3′ slice junction, or a self-circularizing catalytic intron such as a Group I, Group II or Group III Introns. Non-limiting examples of group I intron self-splicing sequences may include self-splicing permuted intron-exon sequences derived from T4 bacteriophage gene td, and the intervening sequence (IVS) rRNA of Tetrahymena.

### Other Circularization Methods

In some embodiments, linear circular polyribonucleotides may include complementary sequences, including either repetitive or nonrepetitive nucleic acid sequences within individual introns or across flanking introns. Repetitive nucleic acid sequence are sequences that occur within a segment of the circular polyribonucleotide. In some embodiments, the circular polyribonucleotide includes a repetitive nucleic acid sequence. In some embodiments, the repetitive nucleotide sequence includes poly CA or poly UG sequences. In some embodiments, the circular polyribonucleotide includes at least one repetitive nucleic acid sequence that hybridizes to a complementary repetitive nucleic acid sequence in another segment of the circular polyribonucleotide, with the hybridized segment forming an internal double strand. In some embodiments, repetitive nucleic acid sequences and complementary repetitive nucleic acid sequences from two separate circular polyribonucleotides hybridize to generate a single circularized polyribonucleotide, with the hybridized segments forming internal double strands. In some embodiments, the complementary sequences are found at the 5′ and 3′ ends of the linear circular polyribonucleotides. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the circular polyribonucleotide. Such methods may include, but are not limited to click chemistry (e.g., alkyne and azide based methods, or clickable bases), olefin metathesis, phosphoramidate ligation, hemiaminal-imine crosslinking, base modification, and any combination thereof.

In some embodiments, enzymatic methods of circularization may be used to generate the circular polyribonucleotide. In some embodiments, a ligation enzyme, e.g., DNA or RNA ligase, may be used to generate a template of the circular polyribonuclease or complement, a complementary strand of the circular polyribonuclease, or the circular polyribonuclease.

Circularization of the circular polyribonucleotide may be accomplished by methods known in the art, for example, those described in “RNA circularization strategies in vivo and in vitro” by Petkovic and Muller from Nucleic Acids Res, 2015, 43(4): 2454-2465, and “In vitro circularization of RNA” by Muller and Appel, from RNA Biol, 2017, 14(8):1018-1027.

The circular polyribonucleotide may encode a sequence and/or motifs useful for replication. Exemplary replication elements are described in paragraphs [0280]-[0286] of WO2019/118919, which is hereby incorporated by reference in its entirety.

### Antigen

The circular polyribonucleotide described herein comprises an antigenic sequence or a sequence encoding an antigen and/or epitope. An antigen or antigenic sequence comprises one or more epitopes. An epitope is part of an antigen or antigenic sequence that is recognized, targeted, or bound by a given antibody or T cell receptor. An epitope can be a linear epitope, for example, a contiguous sequence of nucleic acids or amino acids. An epitope can be a conformational epitope, for example, an epitope that contains amino acids that form an epitope in the folded conformation of the protein. A conformational epitope can contain non-contiguous amino acids from a primary amino acid sequence. As another example, a conformational epitope comprises nucleic acids that form an epitope in the folded conformation of an antigenic sequence based on its secondary structure or tertiary structure.

In some embodiments, an antigen or epitope comprises all or a part of a protein, a peptide, a glycoprotein, a lipoprotein, a phosphoprotein, a ribonucleoprotein, a carbohydrate (e.g., a polysaccharide), a lipid (e.g., a phospholipid or triglyceride), or a nucleic acid (e.g., DNA, RNA).

In other embodiments, an antigen or epitope comprises a protein antigen or epitope (e.g., a peptide antigen or peptide epitope from a protein, glycoprotein, lipoprotein, phosphoprotein, or ribonucleoprotein). An antigen or epitope can comprise an amino acid, a sugar, a lipid, a phosphoryl, or a sulfonyl group, or a combination thereof.

A protein antigen or epitope can comprise a post-translational modification, for example, glycosylation, ubiquitination, phosphorylation, nitrosylation, methylation, acetylation, amidation, hydroxylation, sulfation, or lipidation.

In some embodiments, an epitope comprises or contains at least 4, at least 5, at least 6, at least 7, at least 8, at least 9, at least 10, at least 11, at least 12, at least 13, at least 14, at least 15, at least 16, at least 17, at least 18, at least. 19, at least 20, at least 21, at least 22, at least 23, at least 24, at least 25, at least 26, at least 27, at least 28, at least 29, or at least 30 amino acids, or more. In some embodiments, an epitope comprises or contains at most 4, at most 5, at most 6, at most 7, at most 8, at most 9, at most 10, at most 11, at most 12, at most 13, at most 14, at most 15, at most 16, at most 17, at most 18, at most. 19, at most 20, at most 21, at most 22, at most 23, at most 24, at most 25, at most 26, at most 27, at most 28, at most 29, or at most 30 amino acids, or less. In some embodiments, an epitope comprises or contains 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, an epitope contains 5 amino acids. In some embodiments, an epitope contains 6 amino acids. In some embodiments, an epitope contains 7 amino acids. In some embodiments, an epitope contains 8 amino acids. In some embodiments, an epitope can be about 8 to about 11 amino acids. In some embodiments, an epitope can be about 9 to about 22 amino acids.

The antigens comprise antigens recognized by B cells, antigens recognized by T cells, or a combination thereof. In some embodiments, the antigens comprise antigens recognized by B cells. In some embodiments, the antigens are antigens recognized by B cells. In some embodiments, the antigens comprise antigens recognized by T cells. In some embodiments, the antigens are antigens recognized by T cells.

The epitopes comprise epitopes recognized by B cells, epitopes recognized by T cells, or a combination thereof. In some embodiments, the epitopes comprise epitopes recognized by B cells. In some embodiments, the epitopes are epitopes recognized by B cells. In some embodiments, the epitopes comprise epitopes recognized by T cells. In some embodiments, the epitopes are epitopes recognized by T cells.

Techniques for identifying antigens and epitopes in silico have been disclosed, for example, in Sanchez-Trincado J L, et al. (Fundamentals and methods for T-and B-cell epitope prediction, J. Immunol. Res., 2017:2680160. doi: 10.1155/2017/2680160 (2017)); Grifoni, A, et al. (A Sequence Homology and Bioinformatic Approach Can Predict Candidate Targets for Immune Responses to SARS-CoV-2, Cell Host Microbe, 27(4):671-680 (2020)); Russi R C et al. (In silico prediction of epitopes recognized by T cells and B cells in PmpD: First step towards to the design of a Chlamydia trachomatis vaccine, Biomedical J., 41(2):109-117 (2018)); Baruah V, et al. (Immunoinformatics-aided identification of T cell and B cell epitopes in the surface glycoprotein of 2019-nCoV, J. Med. Virol., 92(5), doi: 10.1002/jmv.25698 (2020)); each of which is incorporated herein by reference in its entirety.

In some embodiments, an antigenic sequence or epitope comprises a polynucleotide. In some embodiments, an antigenic sequence or epitope is a polynucleotide. In some embodiments, an antigenic sequence or epitope comprises an RNA. In some embodiments, an antigenic sequence or epitope is an RNA. In some embodiments, an antigenic sequence or epitope comprises a DNA. In some embodiments, an antigenic sequence or epitope is a DNA. In some embodiments, the polynucleotide is encoded in the circular polyribonucleotide.

A circular polyribonucleotide of the disclosure comprises or encodes any number of antigenic sequences, antigens, and/or epitopes. In a particular embodiment, a circular polyribonucleotide comprises or encodes at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more of antigenic sequences, antigens, and/or epitopes.

In some embodiments, a circular polyribonucleotide comprises or encodes, for example, at most 1, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less of antigenic sequences, antigens, and/or epitopes.

In some embodiments, a circular polyribonucleotide comprises or encodes about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 of antigenic sequences, antigens, and/or epitopes.

A circular polyribonucleotide of the disclosure comprises or encodes one or more epitopes from an antigen or antigenic sequence. In some embodiments, an antigen comprises an amino acid sequence, which contains multiple epitopes (e.g., epitopes recognized by B cells and/or T cells) therein, and a circular polyribonucleotide encoding one or more of those epitopes. In a particular embodiment, an antigenic sequence comprises a polyribonucleotide sequence, which contains multiple epitopes (e.g., epitopes recognized by B cells and/or T cells) therein, and a circular polyribonucleotide comprising one or more of those epitopes.

A circular polyribonucleotide comprises or encode, for example, at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more epitopes from one antigen.

In some embodiments, a circular polyribonucleotide comprises or encodes, for example, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, or at most 500, or less epitopes from one antigen or antigenic sequence.

In some embodiments, a circular polyribonucleotide comprises or encodes, for example, about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 epitopes from one antigen or antigenic sequence.

In some embodiments, a circular polyribonucleotide encodes variants of an antigenic sequence, antigen, and/or epitope. Variants can be naturally-occurring variants (for example, variants identified in sequence data from different viral genera, species, isolates, or quasispecies), or can be derivative sequences as disclosed herein that have been generated in silico (for example, antigen or epitopes with one or more amino acid insertions, deletions, substitutions, or a combination thereof compared to a wild type antigen or epitope).

An antigenic sequence, antigen, and/or epitope is from, for example, a virus, such as a viral surface protein, a viral membrane protein, a viral envelope protein, a viral capsid protein, a viral nucleocapsid protein, a viral spike protein, a viral entry protein, a viral membrane fusion protein, a viral structural protein, a viral non-structural protein, a viral regulatory protein, a viral accessory protein, a secreted viral protein, a viral polymerase protein, a viral DNA polymerase, a viral RNA polymerase, a viral protease, a viral glycoprotein, a viral fusogen, a viral helical capsid protein, a viral icosahedral capsid protein, a viral matrix protein, a viral replicase, a viral transcription factor, or a viral enzyme.

In some embodiments, the antigenic sequence, antigen, and/or epitope is from one of these viruses:

Orthomyxovirus: Useful antigens can be from an influenza A, B or C virus, such as the hemagglutinin, neuraminidase or matrix M2 proteins. Where the antigen is an influenza A virus hemagglutinin it may be from any subtype e.g. HI, H2, H3, H4, H5, H6, H7, H8, H9, H10, H11, H12, H13, H14, H15 or H16.

Paramyxoviridae viruses: Viral antigens include, but are not limited to, those derived from Pneumoviruses (e.g. respiratory syncytial virus (RSV)), Rubulaviruses (e.g. mumps virus), Paramyxoviruses (e.g. parainfluenza virus), Metapneumoviruses and Morbilliviruses (e.g. measles virus), Henipaviruses (e.g. Nipah virus).

Poxviridae: Viral antigens include, but are not limited to, those derived from Orthopoxvirus such as Variola vera, including but not limited to, Variola major and Variola minor.

Picornavirus: Viral antigens include, but are not limited to, those derived from Picornaviruses, such as Enteroviruses, Rhinoviruses, Heparnavirus, Cardioviruses and Aphthoviruses. In one embodiment, the enterovirus is a poliovirus e.g. a type 1, type 2 and/or type 3 poliovirus. In another embodiment, the enterovirus is an EV71 enterovirus. In another embodiment, the enterovirus is a coxsackie A or B virus.

Bunyavirus: Viral antigens include, but are not limited to, those derived from an Orthobunyavirus, such as California encephalitis virus, a Phlebovirus, such as Rift Valley Fever virus, or a Nairovirus, such as Crimean-Congo hemorrhagic fever virus.

Heparnavirus: Viral antigens include, but are not limited to, those derived from a Heparnavirus, such as hepatitis A virus (HAV).

Filovirus: Viral antigens include, but are not limited to, those derived from a Filovirus, such as an Ebola virus (including a Zaire, Ivory Coast, Reston or Sudan ebolavirus) or a Marburg virus.

Togavirus: Viral antigens include, but are not limited to, those derived from a Togavirus, such as a Rubivirus, an Alphavirus, or an Arterivirus. This includes rubella virus.

Flavivirus: Viral antigens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus, Zika virus.

Pestivirus: Viral antigens include, but are not limited to, those derived from a Pestivirus, such as Bovine viral diarrhea (BVDV), Classical swine fever (CSFV) or Border disease (BDV).

Hepadnavirus: Viral antigens include, but are not limited to, those derived from a Hepadnavirus, such as Hepatitis B virus. The hepatitis B virus antigen may be a hepatitis B virus surface antigen (HBsAg).

Other hepatitis viruses: Viral antigens include, but are not limited to, those derived from a hepatitis C virus, delta hepatitis virus, hepatitis E virus, or hepatitis G virus.

Rhabdovirus: Viral antigens include, but are not limited to, those derived from a Rhabdovirus, such as a Lyssavirus {e.g. a Rabies virus) and Vesiculovirus (VSV).

Caliciviridae: Viral antigens include, but are not limited to, those derived from Calciviridae, such as Norwalk virus (Norovirus), and Norwalk-like Viruses, such as Hawaii Virus and Snow Mountain Virus.

Retrovirus: Viral antigens include, but are not limited to, those derived from an Oncovirus, a Lentivirus (e.g. HIV-1 or HIV-2) or a Spumavirus.

Reovirus: Viral antigens include, but are not limited to, those derived from an Orthoreovirus, a Rotavirus, an Orbivirus, or a Coltivirus.

Parvovirus: Viral antigens include, but are not limited to, those derived from Parvovirus B19.

Bocavirus: Viral antigens include, but are not limited to, those derived from bocavirus.

Herpesvirus: Viral antigens include, but are not limited to, those derived from a human herpesvirus, such as, by way of example only, Herpes Simplex Viruses (HSV) (e.g. HSV types 1 and 2), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8).

Papovaviruses: Viral antigens include, but are not limited to, those derived from Papillomaviruses and Polyomaviruses. The (human) papillomavirus may be of serotype 1, 2, 4, 5, 6, 8, 11, 13, 16, 18, 31, 33, 35, 39, 41, 42, 47, 51, 57, 58, 63 or 65 e.g. from one or more of serotypes 6, 11, 16 and/or 18.

Orthohantaviruses: Viral antigens include, but are not limited to, those derived from hantaviruses.

Arenavirus: Viral antigens include, but are not limited to, those derived from Guanarito virus, Junin virus, Lassa virus, Lujo virus, Machupo virus, Sabia virus, or Whitewater Arroyo virus.

Adenovirus: Viral antigens include those derived from adenovirus serotype 36 (Ad-36).

Community acquired respiratory viruses: Viral antigens include those derived from community acquired respiratory viruses.

Coronavirus: Viral antigens include, but are not limited to, those derived from a SARS coronavirus (e.g., SARS-CoV-1 and SARS-CoV-2), MERS coronavirus, avian infectious bronchitis (IBV), Mouse hepatitis virus (MHV), and Porcine transmissible gastroenteritis virus (TGEV). The coronavirus antigen may be a spike polypeptide.

In some embodiments, an antigenic sequence, antigen, and/or epitope is from a coronavirus, for example, a severe acute respiratory syndrome associated coronavirus (SARS-CoV, e.g., SARS-CoV-1, SARS-CoV-2), a Middle East respiratory syndrome coronavirus (MERS-CoV), or another coronavirus. In some embodiments, an antigen and/or epitope is from a predicted open reading frame from a coronavirus genome.

New SARS isolates may be identified by a percent homology of 99%, 98%, 97%, 95%, 92%, 90%, 85%, or 80% homology of the polynucleotide sequence for specific genomic regions for the new virus with the polynucleotide sequence for specific genomic regions of the known SARS viruses. Additionally, new SARS isolates may be identified by a percent homology of 99%, 98%, 97%, 95%, 92%, 90%, 85%, or 80% homology of the polypeptide sequence encoded by the polynucleotide of specific genomic regions of the new SARS virus to the polypeptide sequence encoded by the polynucleotides of specific regions of the known SARS virus. These genomic regions may include regions (e.g., gene products or ORFs) which are typically in common among numerous coronaviruses, as well as group specific regions (e.g., antigenic groups), such as, for example, any one of the following genomic regions which could be readily identified by a virologist skilled in the art: 5′untranslated region (UTR), leader sequence, ORF1a, ORF1b, nonstructural protein 2 (NS2), hemagglutinin-esterase glycoprotein (HE) (also referred to as E3), spike glycoprotein (S) (also referred to as E2), ORF3a, ORF3b, nonstructural protein 4 (NS4), envelope (small membrane) protein (E) (also referred to as sM), membrane glycoprotein (M) (also referred to as E1), ORF5a, ORF5b, nucleocapsid phosphoprotein (N), ORF6, ORF7a, ORF7b, ORF8, ORF8a, ORF8b, ORF9a, ORF9b, ORF10, intergenic sequences, receptor binding domain (RBD) of a spike protein, 3′UTR, or RNA dependent RNA polymerase (pol). The SARS virus may have identifiable genomic regions with one or more the above-identified genomic regions. A SARS viral antigen includes a protein encoded by any one of these genomic regions. A SARS viral antigen may be a protein or a fragment thereof, which is highly conserved with coronaviruses. A SARS viral antigen may be a protein or fragment thereof, which is specific to the SARS virus (as compared to known coronaviruses).

In some embodiments, an antigenic sequence, antigen, and/or epitope is from a predicted transcript from a SARS-CoV genome. In some embodiments, an antigenic sequence, antigen, and/or epitope is from a protein encoded by or is the nucleic acid encoding an open reading frames from a SARS-CoV genome. Non-limiting examples of open reading frames in SARS-CoV genomes can include ORF1a, ORF1b, spike (S), ORF3a, ORF3b, envelope (E), membrane (M), ORF6, ORF7a, ORF7b, ORF8, ORF8a, ORF8b, ORF9a, ORF9b, nucleocapsid (N), and ORF10. ORF1a and ORF1b encodes 16 non-structural proteins (nsp), for example, nsp1, nsp2, nsp3, nsp4, nsp5, nsp6, nsp7, nsp8, nsp9, nsp10, nsp11, nsp12, nsp13, nsp14, nsp15, and nsp16. Nonstructural proteins, for example, contribute to viral replication, viral assembly, immune response modulation, or a combination thereof In some embodiments, the antigen is a non-structural protein or is an antigenic sequence encoding a non-structural protein. In some embodiments, epitopes are from a coronavirus non-structural protein.

Spike (S) encodes a spike protein, which in some embodiments contributes to binding to a host cell receptor, fusion of the virus with the host cell membrane, entry of the virus into a host cell, or a combination thereof. In some embodiments, the antigen is a spike protein. In some embodiments, antigenic sequences, antigens, and/or epitopes are from a spike protein. In some embodiments, the antigen is a receptor binding domain (RBD) of a spike protein.

Envelope (E) encodes envelope protein, which in some embodiments contributes to virus assembly and morphogenesis. In some embodiments, the antigen is an envelope protein. In some embodiments, antigenic sequences, antigens, and/or epitopes are from a coronavirus envelope protein.

Membrane (M) encodes membrane protein, which in some embodiments contributes to viral assembly. In some embodiments, the antigen is a membrane protein. In some embodiments, antigenic sequences, antigens, and/or epitopes are from a coronavirus membrane protein.

Nucleocapsid (N) encodes nucleocapsid protein, which in some embodiments form complexes with genomic RNA and contribute to viral assembly, and/or interact with M protein. In some embodiments, the antigen is a nucleocapsid protein. In some embodiments, antigenic sequences, antigens, and/or epitopes are from a coronavirus nucleocapsid protein.

ORF3a, ORF3b, ORF6, ORF7a, ORF7b, ORF8, ORF8a, ORF8b, ORF9a, ORF9b, and ORF10 encode accessory proteins. In some embodiments, accessory proteins modulate host cell signaling, modulate host cell immune responses, are incorporated into mature virions as minor structural proteins, or a combination thereof. In some embodiments, the antigen is an accessory protein. In some embodiments, antigenic sequences, antigens, and/or epitopes are from a coronavirus accessory protein.

In some embodiments, antigenic sequences, antigens, and/or epitopes are from a spike protein. In some embodiments, antigenic sequences, antigens, and/or epitopes comprise a receptor binding domain of a Spike protein. In some embodiments, antigenic sequences, antigens, and/or epitopes comprise an ACE2 binding domain of a Spike protein. In some embodiments, antigenic sequences, antigens, and/or epitopes comprise an S1 subunit Spike protein, an S2 subunit of spike protein, or a combination thereof. In some embodiments, antigens and/or epitopes comprise an ectodomain of a spike protein. In some embodiments, antigenic sequences, antigens, and/or epitopes comprises Gln498, Thr500, Asn501, or a combination thereof from a coronavirus spike protein. In some embodiments, an antigen and/or epitope of the disclosure comprises Lys417, Tyr453, or a combination thereof from a coronavirus spike protein. In some embodiments, an antigen and/or epitope of the disclosure comprises Gln474, Phe486, or a combination thereof from a coronavirus spike protein. In some embodiments, an antigen and/or epitope of the disclosure comprises Gln498, Thr500, Asn501, Lys417, Tyr453, Gln474, Phe486, one or more equivalent amino acids from a spike protein variant or derivative, or a combination thereof from a coronavirus spike protein.

In some embodiments, antigenic sequences, antigens, and/or epitopes are encoded by or derived from ORF1a. In some embodiments, antigenic sequences, antigens, and/or epitopes are encoded by or derived from a SARS-CoV ORF1b. In some embodiments, antigenic sequences, antigens, and/or epitopes are encoded by or derived from a SARS-CoV spike. In some embodiments, antigenic sequences, antigens, and/or epitopes are encoded by or derived from a SARS-CoV ORF3a. In some embodiments, antigenic sequences, antigens, and/or epitopes are encoded by or derived from a SARS-CoV ORF3b. In some embodiments, antigenic sequences, antigens, and/or epitopes are encoded by or derived from a SARS-CoV envelope (E). In some embodiments, antigenic sequences, antigens, and/or epitopes are encoded by or derived from a SARS-CoV membrane (M). In some embodiments, antigenic sequences, antigens, and/or epitopes are encoded by or derived from a SARS-CoV ORF6. In some embodiments, antigenic sequences, antigens, and/or epitopes are encoded by or derived from a SARS-CoV ORF7a. In some embodiments, antigenic sequences, antigens, and/or epitopes are encoded by or derived from a SARS-CoV ORF7b. In some embodiments, antigenic sequences, antigens, and/or epitopes are encoded by or derived from a SARS-CoV ORF8. In some embodiments, antigenic sequences, antigens, and/or epitopes are encoded by or derived from a SARS-CoV ORF8a. In some embodiments, antigenic sequences, antigens, and/or epitopes are encoded by or derived from a SARS-CoV ORF9a. In some embodiments, antigenic sequences, antigens, and/or epitopes are encoded by or derived from a SARS-CoV ORF9b. In some embodiments, antigenic sequences, antigens, and/or epitopes are encoded by or derived from a SARS-CoV nucleocapsid (N). In some embodiments, antigenic sequences, antigens, and/or epitopes are encoded by or derived from a SARS-CoV ORF10. In some embodiments, antigenic sequences, antigens, and/or epitopes are encoded by or derived from a SARS-CoV spike (S), envelope (E), membrane (M), and nucleocapsid (N).

In some embodiments, antigenic sequences, antigens, and/or epitopes are encoded by or derived from a SARS-CoV spike (S), envelope (E), and membrane (M). In some embodiments, antigenic sequences, antigens, and/or epitopes are encoded by or derived from a SARS-CoV spike (S), envelope (E), and nucleocapsid (N). In some embodiments, antigenic sequences, antigens, and/or epitopes are encoded by or derived from a SARS-CoV spike (S), membrane (M), and nucleocapsid (N). In some embodiments, antigenic sequences, antigens, and/or epitopes are encoded by or derived from a SARS-CoV envelope (E), membrane (M), and nucleocapsid (N).

In some embodiments, antigenic sequences, antigens, and/or epitopes are encoded by or derived from a SARS-CoV spike (S) and envelope (E). In some embodiments, antigenic sequences, antigens, and/or epitopes are encoded by or derived from a SARS-CoV spike (S) and membrane (M). In some embodiments, antigenic sequences, antigens, and/or epitopes are encoded by or derived from a SARS-CoV spike (S) and nucleocapsid (N). In some embodiments, antigenic sequences, antigens, and/or epitopes are encoded by or derived from a SARS-CoV envelope (E) and membrane (M). In some embodiments, antigenic sequences, antigens, and/or epitopes are encoded by or derived from a SARS-CoV envelope (E) and nucleocapsid (N). In some embodiments, antigenic sequences, antigens, and/or epitopes are encoded by or derived from a SARS-CoV membrane (M) and nucleocapsid (N).

In some embodiments, antigenic sequences, antigens, and/or epitopes are encoded by or derived from a SARS-CoV spike (S), envelope (E), membrane (M), and nucleocapsid (N), ORF3a, ORF6, ORF7a, ORF7b, ORF7b, ORF8, and ORF10.

In some embodiments, antigenic sequences, antigens, and/or epitopes are not encoded by or derived from a SARS-CoV ORF1a. In some embodiments, antigenic sequences, antigens, and/or epitopes are not encoded by or derived from a SARS-CoV ORF1b. In some embodiments, antigenic sequences, antigens, and/or epitopes are not encoded by or derived from a SARS-CoV spike. In some embodiments, antigenic sequences, antigens, and/or epitopes are not encoded by or derived from a SARS-CoV ORF3a. In some embodiments, antigenic sequences, antigens, and/or epitopes are not encoded by or derived from a SARS-CoV ORF3b. In some embodiments, antigenic sequences, antigens, and/or epitopes are not encoded by or derived from a SARS-CoV envelope (E). In some embodiments, antigenic sequences, antigens, and/or epitopes are not encoded by or derived from a SARS-CoV membrane (M). In some embodiments, antigenic sequences, antigens, and/or epitopes are not encoded by or derived from a SARS-CoV ORF6. In some embodiments, antigenic sequences, antigens, and/or epitopes not encoded by or derived from a SARS-CoV ORF7a. In some embodiments, antigenic sequences, antigens, and/or epitopes are not encoded by or derived from a SARS-CoV ORF7b. In some embodiments, antigenic sequences, antigens, and/or epitopes are not encoded by or derived from a SARS-CoV ORF8. In some embodiments, antigenic sequences, antigens, and/or epitopes are not encoded by or derived from a SARS-CoV ORF8a. In some embodiments, antigenic sequences, antigens, and/or epitopes are not encoded by or derived from a SARS-CoV ORF9a. In some embodiments, antigenic sequences, antigens, and/or epitopes are not encoded by or derived from a SARS-CoV ORF9b. In some embodiments, antigenic sequences, antigens, and/or epitopes are not encoded by or derived from a SARS-CoV nucleocapsid (N). In some embodiments, antigenic sequences, antigens, and/or epitopes are not encoded by or derived from a SARS-CoV ORF10. In some embodiments, antigenic sequences, antigens, and/or epitopes are not encoded by or derived from a SARS-CoV spike (S), envelope (E), membrane (M), and nucleocapsid (N).

In a particular embodiment, an antigenic sequence, antigen, and/or epitope is encoded by or derived from SARS-CoV-2.

A non-limiting example of a SARS-CoV-2 genome is provided in DB Source accession MN908947.3, the complete genome sequence of a Severe acute respiratory syndrome coronavirus 2 isolate, the content of which is incorporated herein by reference in its entirety. DB Source accession MN908947.3: 21563-25384 correspond to the S protein, the content of which is incorporated herein by reference in its entirety. A non-limiting example of a SARS-CoV-2 spike protein is provided in GenBank Sequence: QHD43416.1, the sequence of a spike protein of a Severe acute respiratory syndrome coronavirus 2 isolate, the content of which is incorporated herein by reference in its entirety.

A non-limiting example of a SARS-CoV-2 genome is provide in sequence NCBI Reference Sequence accession number NC_045512, version NC_045512.2, the complete genome sequence of Severe acute respiratory syndrome coronavirus 2 isolate Wuhan-Hu-1, the content of which is incorporated herein by reference in its entirety.

A non-limiting example of a SARS-CoV-2 genome is provided in sequence NCBI Reference Sequence accession number MW450666, the complete genome sequence of Severe acute respiratory syndrome coronavirus 2 isolate, the content of which is incorporated herein by reference in its entirety.

A non-limiting example of a SARS-CoV-2 genome is provided in sequence NCBI Reference Sequence accession number MW487270, the complete genome sequence of Severe acute respiratory syndrome coronavirus 2 lineage B.1.1.7 virus, the content of which is incorporated herein by reference in its entirety.

A non-limiting example of a SARS-CoV-2 genome is provided in sequence GISAID Reference Sequence accession number EPI_ISL_792683, the complete genome sequence of Severe acute respiratory syndrome coronavirus 2 lineage P.1 virus, the content of which is incorporated herein by reference in its entirety.

A non-limiting example of a SARS-CoV-2 genome is provided in sequence GISAID Reference Sequence accession number EPI_ISL_678615, the complete genome sequence of Severe acute respiratory syndrome coronavirus 2 lineage B.1.351 virus, the content of which is incorporated herein by reference in its entirety.

Non-limiting examples of a SARS-CoV-2 genome are provided in sequence NCBI Reference Sequence accession numbers MW972466-MW974550, the complete genome sequence of Severe acute respiratory syndrome coronavirus 2 lineage B.1.427 and B.1.429 virus, the contents of which are incorporated herein by reference in their entirety.

Non-limiting examples of a SARS-CoV-2 genome are provided in sequence NCBI Reference Sequence accession numbers MZ156756-MZ226428, the complete genome sequence of Severe acute respiratory syndrome coronavirus 2 virus, the contents of which are incorporated herein by reference in their entirety.

In some embodiments, an antigen and/or epitope is from a predicted transcript from a SARS-CoV-2 genome. In some embodiments, an antigen and/or epitope is from a protein encoded by an open reading frames from a SARS-CoV-2 genome, or a derivative thereof. Non-limiting examples of open reading frames in the SARS-CoV-2 genome include ORF1a, ORF1b, spike (S), ORF3a, envelope (E), membrane (M), ORF6, ORF7a, ORF7b, ORF8, nucleocapsid (N), and ORF10. In some embodiments, a SARS-CoV-2 genome encodes an ORF3b, ORF9a, ORF9b, or a combination thereof. In some embodiments, a SARS-CoV-2 genome does not encode an ORF3b, ORF9a, ORF9b, or any combination thereof.

Nonlimiting examples of amino acid sequences are provided in TABLE 1. In some embodiments, the antigen comprises a sequence having at least about 80%, 85%, 90%, 95%, 97%, 98%, 99%, or 100% sequence identity to a sequence from Table 1.

TABLE 1
examples of amino acid sequence of proteins
encoded by a SARS-CoV2 genome.
TABLE 1

| SEQ | | |
|---|---|---|
| ID | Descrip- | |
| NO: | tion | Sequence |
| 1 | Spike | MFVFLVLLPLVSSQCVNLTTRTQLPPAYTNSFT |
| | (S) | RGVYYPDKVFRSSVLHSTQDLFLPFFSNVTWFH |
| | protein | AIHVSGTNGTKRFDNPVLPFNDGVYFASTEKSN |
| | | IIRGWIFGTTLDSKTQSLLIVNNATNVVIKVCE |
| | | FQFCNDPFLGVYYHKNNKSWMESEFRVYSSANN |
| | | CTFEYVSQPFLMDLEGKQGNFKNLREFVFKNID |
| | | GYFKIYSKHTPINLVRDLPQGFSALEPLVDLPI |
| | | GINITRFQTLLALHRSYLTPGDSSSGWTAGAAA |
| | | YYVGYLQPRTFLLKYNENGTITDAVDCALDPLS |
| | | ETKCTLKSFTVEKGIYQTSNFRVQPTESIVRFP |
| | | NITNLCPFGEVFNATRFASVYAWNRKRISNCVA |
| | | DYSVLYNSASFSTFKCYGVSPTKLNDLCFTNVY |
| | | ADSFVIRGDEVRQIAPGQTGKIADYNYKLPDDF |
| | | TGCVIAWNSNNLDSKVGGNYNYLYRLFRKSNLK |
| | | PFERDISTEIYQAGSTPCNGVEGFNCYFPLQSY |
| | | GFQPTNGVGYQPYRVVVLSFELLHAPATVCGPK |
| | | KSTNLVKNKCVNFNFNGLTGTGVLTESNKKFLP |
| | | FQQFGRDIADTTDAVRDPQTLEILDITPCSFGG |
| | | VSVITPGTNTSNQVAVLYQDVNCTEVPVAIHAD |
| | | QLTPTWRVYSTGSNVFQTRAGCLIGAEHVNNSY |
| | | ECDIPIGAGICASYQTQTNSPRRARSVASQSII |
| | | AYTMSLGAENSVAYSNNSIAIPTNFTISVTTEI |
| | | LPVSMTKTSVDCTMYICGDSTECSNLLLQYGSF |
| | | CTQLNRALTGIAVEQDKNTQEVFAQVKQIYKTP |
| | | PIKDFGGFNFSQILPDPSKPSKRSFIEDLLFNK |
| | | VTLADAGFIKQYGDCLGDIAARDLICAQKFNGL |
| | | TVLPPLLTDEMIAQYTSALLAGTITSGWTFGAG |
| | | AALQIPFAMQMAYRFNGIGVTQNVLYENQKLIA |
| | | NQFNSAIGKIQDSLSSTASALGKLQDVVNQNAQ |
| | | ALNTLVKQLSSNFGAISSVLNDILSRLDKVEAE |
| | | VQIDRLITGRLQSLQTYVTQQLIRAAEIRASAN |
| | | LAATKMSECVLGQSKRVDFCGKGYHLMSFPQSA |
| | | PHGVVFLHVTYVPAQEKNFTTAPAICHDGKAHF |
| | | PREGVFVSNGTHWFVTQRNFYEPQIITTDNTFV |
| | | SGNCDVVIGIVNNTVYDPLQPELDSFKEELDKY |
| | | FKNHTSPDVDLGDISGINASVVNIQKEIDRLNE |
| | | VAKNLNESLIDLQELGKYEQYIKWPWYIWLGFI |
| | | AGLIAIVMVTIMLCCMTSCCSCLKGCCSCGSCC |
| | | KFDEDDSEPVLKGVKLHYT |
| 2 | Envelope | MYSFVSEETGTLIVNSVLLFLAFVVFLLVTLAI |
| | (E) | NLTALRLCAYCCIVNVSLVKPSFYVYSRVKNLN |
| | protein | SSRVPDLLV |
| 3 | Membrane | MADSNGTITVEELKKLLEQWNLVIGFLFLTWIC |
| | (M) | LLQFAYANRNRFLYIIKLIFLWLLWPVTLACFV |
| | protein | LAAVYRINWITGGIAIAMACLVGLMWLSYFIAS |
| | | FRLFARTRSMWSFNPETNILLNVPLHGTILTRP |
| | | LLESELVIGAVILRGHLRIAGHHLGRCDIKDLP |
| | | KEITVATSRTLSYYKLGASQRVAGDSGFAAYSR |
| | | YRIGNYKLNTDHSSSSDNIALLVQ |
| 4 | Nucleo- | MSDNGPQNQRNAPRITFGGPSDSTGSNQNGERS |
| | capsid | GARSKQRRPQGLPNNTASWFTALTQHGKEDLKF |
| | (N) | PRGQGVPINTNSSPDDQIGYYRRATRRIRGGDG |
| | protein | KMKDLSPRWYFYYLGTGPEAGLPYGANKDGIIW |
| | | VATEGALNTPKDHIGTRNPANNAAIVLQLPQGT |
| | | TLPKGFYAEGSRGGSQASSRSSSRSRNSSRNST |
| | | PGSSRGTSPARMAGNGGDAALALLLLDRLNQLE |
| | | SKMSGKGQQQQGQTVTKKSAAEASKKPRQKRTA |
| | | TKAYNVTQAFGRRGPEQTQGNFGDQELIRQGTD |
| | | YKHWPQIAQFAPSASAFFGMSRIGMEVTPSGTW |
| | | LTYTGAIKLDDKDPNFKDQVILLNKHIDAYKTF |
| | | PPTEPKKDKKKKADETQALPQRQKKQQTVTLLP |
| | | AADLDDFSKQLQQSMSSADSTQA |
| 5 | ORF3a | MDLFMRIFTIGTVTLKQGEIKDATPSDFVRATA |
| | accessory | TIPIQASLPFGWLIVGVALLAVFQSASKIITLK |
| | protein | KRWQLALSKGVHFVCNLLLLFVTVYSHLLLVAA |
| | | GLEAPFLYLYALVYFLQSINFVRIIMRLWLCWK |
| | | CRSKNPLLYDANYFLCWHTNCYDYCIPYNSVTS |
| | | SIVITSGDGTTSPISEHDYQIGGYTEKWESGVK |
| | | DCVVLHSYFTSDYYQLYSTQLSTDTGVEHVTFF |
| | | IYNKIVDEPEEHVQIHTIDGSSGVVNPVMEPIY |
| | | DEPTTTTSVPL |
| 6 | ORF6 | MFHLVDFQVTIAEILLIIMRTFKVSIWNLDYII |
| | accessory | NLIIKNLSKSLTENKYSQLDEEQPMEID |
| | protein | |
| 7 | ORF7a | MKIILFLALITLATCELYHYQECVRGTTVLLKE |
| | accessory | PCSSGTYEGNSPFHPLADNKFALTCFSTQFAFA |
| | protein | CPDGVKHVYQLRARSVSPKLFIRQEEVQELYSP |
| | | IFLIVAAIVFITLCFTLKRKTE |
| 8 | ORF7b | MIELSLIDFYLCFLAFLLFLVLIMLIIFWFSLE |
| | accessory | LQDHNETCHA |
| | protein | |
| 9 | ORF8 | MKFLVFLGIITTVAAFHQECSLQSCTQHQPYVV |
| | accessory | DDPCPIHFYSKWYIRVGARKSAPLIELCVDEAG |
| | protein | SKSPIQYIDIGNYTVSCLPFTINCQEPKLGSLV |
| | | VRCSFYEDFLEYHDVRVVLDFI |
| 10 | ORF10 | MGYINVFAFPFTIYSLLLCRMNSRNYIAQVDVV |
| | accessory | NFNLT |
| | protein | |

Additional non-limiting examples proteins encoded by a SARS-CoV-2 genome include those with the contents of NCBI accession numbers MT334522, MT334523, MT334524, MT334525, MT334526, MT334527, MT334528, MT334529, MT334530, MT334531, MT334532, MT334533, MT334534, MT334535, MT334536, MT334537, MT334538, MT334539, MT334540, MT334541, MT334542, MT334543, MT334544, MT334545, MT334546, MT334555, MT334547, MT334548, MT334549, MT334550, MT334551, MT334552, MT334553, MT334554, MT334556, MT334557, MT334558, MT334559, MT334560, MT334561, MT334562, MT334563, MT334564, MT334565, MT334566, MT334567, MT334568, MT334569, MT334570, MT334571, MT334572, MT334573, MT326097, MT326106, MT326107, MT326116, MT326117, MT326124, MT326125, MT326126, MT326127, MT326134, MT326135, MT326136, MT326137, MT326138, MT326139, MT326140, MT326141, MT326142, MT326143, MT326144, MT326145, MT326146, MT326148, MT326149, MT326150, MT326151, MT326152, MT326158, MT326159, MT326160, MT326161, MT326162, MT326168, MT326169, MT326170, MT326171, MT326172, MT326178, MT326179, MT326180, MT326181, MT326182, MT326183, MT326188, MT326189, MT326190, MT326191, MT326129, MT326121, MT326120, MT326119, MT326118, MT326111, MT326023, MT326025, MT326033, MT326035, MT326036, MT326040, MT326043, MT326045, MT326053, MT326055, MT326056, MT326063, MT326066, MT326070, MT326071, MT326072, MT326075, MT326076, MT326078, MT326079, MT326089, MT325563, MT325565, MT325566, MT326155, MT326163, MT326177, MT326130, MT326128, MT326110, MT326109, MT326108, MT326101, MT326100, MT326099, MT326098, MT326094, MT326093, MT326092, MT325568, MT325569, MT325590, MT325640, MT325606, MT325607, MT325608, MT325609, MT325610, MT325611, MT325616, MT325618, MT325619, MT325620, MT325622, MT325623, MT325624, MT325599, MT325600, MT325601, MT325602, MT325612, MT325613, MT325615, MT325617, MT325625, MT324062, MT324684, MT325573, MT325574, MT325577, MT325579, MT325586, MT325592, MT325593, MT325594, MT325598, MT325605, MT325626, MT325627, MT325633, MT325634, MT326028, MT326031, MT326091, MT326090, MT326085, MT326084, MT326083, MT326082, MT326081, MT326080, MT326077, MT326067, MT326057, MT326024, MT326026, MT326027, MT326032, MT326034, MT326037, MT326039, MT326041, MT326042, MT326044, MT326046, MT326047, MT326049, MT326050, MT326051, MT326052, MT326054, MT326059, MT326060, MT326061, MT326062, MT326064, MT326065, MT326068, MT326069, MT326073, MT326074, MT326088, MT327745, MT324679, MT325561, MT325571, MT325572, MT325575, MT325583, MT325587, MT325588, MT325589, MT325596, MT325597, MT325603, MT325604, MT325614, MT325621, MT325629, MT325630, MT325631, MT325632, MT325635, MT325636, MT325637, MT325638, MT325639, MT326086, MT326096, MT326102, MT326104, MT326105, MT326112, MT326113, MT326114, MT326115, MT326122, MT328034, MT325564, MT325567, MT326164, MT326165, MT326173, MT326174, MT326184, MT326185, MT326186, MT326187, MT325584, MT325585, MT326087, MT326095, MT326103, MT326123, MT326131, MT326132, MT326133, MT328033, MT325562, MT326147, MT326153, MT326154, MT326156, MT326157, MT326166, MT326167, MT326175, MT326176, MT324680, MT325570, MT325576, MT325578, MT325580, MT325581, MT325582, MT325591, MT325595, MT325628, MT326029, MT326030, MT326038, MT326048, MT326058, MT324681, MT324682, MT324683, MT328032, MT328035, MT322404, MT039874, MT322398, MT322409, MT322421, MT322423, MT322408, MT322413, MT322417, MT322394, MT322407, MT322418, MT322424, MT322411, MT077125, MT322395, MT322396, MT322397, MT322399, MT322400, MT322401, MT322402, MT322403, MT322405, MT322406, MT322414, MT322416, MT322419, MT322420, MT322410, MT322412, MT322415, MT322422, MT320538, MT320891, MT308692, MT308693, MT308695, MT308696, MT308698, MT308699, MT308701, MT308703, MT308704, MT308694, MT308697, MT308700, MT308702, MT293547, MT304476, MT304474, MT304475, MT304477, MT304478, MT304479, MT304481, MT304482, MT304484, MT304485, MT304486, MT304487, MT304488, MT304491, MT304480, MT304483, MT304489, MT304490, MT300186, MT292571, MT292576, MT292578, MT293186, MT292570, MT292573, MT293173, MT292575, MT293179, MT293180, MT293184, MT293189, MT293192, MT293193, MT293194, MT293201, MT293202, MT292572, MT292577, MT293185, MT293187, MT293188, MT291826, MT291832, MT291833, MT291835, MT291836, MT291831, MT293170, MT292574, MT293178, MT293181, MT293183, MT293195, MT293196, MT293197, MT293203, MT293204, MT293223, MT293212, MT293214, MT293215, MT293216, MT293219, MT293224, MT293225, MT293206, MT293208, MT293209, MT293221, MT295464, MT293160, MT293166, MT293171, MT293190, MT293161, MT293167, MT293168, MT293174, MT293175, MT293182, MT293191, MT293158, MT293162, MT293163, MT293164, MT293156, MT293157, MT293159, MT291834, MT291829, MT291827, MT291830, MT291828, MT293169, MT293200, MT293210, MT293211, MT293217, MT293218, MT295465, MT293198, MT293205, MT293207, MT293213, MT293220, MT293222, MT292581, MT292569, MT293172, MT293177, MT293176, MT293199, MT292580, MT292582, MT293165, MT292579, MT273658, MT281577, MT281530, MT276597, MT276598, MT276323, MT276328, MT276331, MT276329, MT276330, MT276324, MT276325, MT276327, MT276326, MT263388, MT263392, MT262900, MT262902, MT262906, MT262908, MT262912, MT262913, MT262914, MT262993, MT263074, MT263381, MT263391, MT262901, MT262903, MT262907, MT262909, MT262911, MT262899, MT262904, MT262915, MT262916, MT262897, MT262898, MT262905, MT262910, MT263400, MT263382, MT263383, MT263384, MT263385, MT262896, MT263407, MT263415, MT263406, MT263408, MT263422, MT263469, MT263439, MT263457, MT263459, MT263432, MT263450, MT263458, MT263467, MT263401, MT263411, MT263413, MT263426, MT263421, MT263443, MT263412, MT263416, MT263417, MT263423, MT263431, MT263461, MT263410, MT263424, MT263425, MT263427, MT263442, MT263402, MT263405, MT263409, MT263418, MT263419, MT263398, MT263399, MT263403, MT263404, MT263414, MT263430, MT263390, MT263434, MT263436, MT263446, MT263448, MT263452, MT263453, MT263456, MT263462, MT263463, MT263386, MT263387, MT263389, MT263428, MT263429, MT263433, MT263435, MT263437, MT263438, MT263440, MT263447, MT263449, MT263455, MT263444, MT263445, MT263451, MT263466, MT263420, MT263441, MT263454, MT263464, MT263465, MT263468, MT263460, MT263393, MT263394, MT263395, MT263396, MT263397, MT259226, MT259275, MT259276, MT259279, MT259247, MT258377, MT258378, MT258379, MT259231, MT259228, MT259238, MT259248, MT256917, MT259227, MT259236, MT256918, MT258380, MT259235, MT259237, MT259239, MT259281, MT259282, MT259283, MT259240, MT259243, MT259249, MT259250, MT259251, MT259256, MT259258, MT259266, MT259267, MT259274, MT259286, MT259287, MT259241, MT259242, MT258381, MT259257, MT259261, MT259262, MT259263, MT259264, MT259268, MT259269, MT259270, MT259271, MT259272, MT259273, MT259277, MT259278, MT259280, MT258383, MT258382, MT259246, MT256924, MT259244, MT259245, MT259252, MT259253, MT259254, MT259255, MT259259, MT259284, MT259229, MT259230, MT259265, MT259260, MT259285, LC534419, LC534418, MT253710, MT253709, MT253705, MT253708, MT253701, MT253702, MT253703, MT253704, MT253706, MT253707, MT251972, MT251974, MT251975, MT251973, MT251976, MT251979, MT253697, MT253699, MT253696, MT253698, MT253700, MT251977, MT251978, MT251980, MT246451, MT246461, MT246471, MT246472, MT246474, MT246483, MT246450, MT246453, MT246454, MT246462, MT246463, MT246464, MT246470, MT246473, MT246480, MT246484, MT246449, MT246455, MT246456, MT246478, MT246485, MT246488, MT246452, MT246460, MT246465, MT246481, MT246482, MT246490, MT246459, MT246468, MT246475, MT246477, MT246479, MT246457, MT246458, MT246466, MT246467, MT246469, MT246476, MT246486, MT246487, MT246489, MT233526, MT246667, MT240479, MT232870, MT232871, MT233523, MT232869, MT232872, MT233519, MT233521, MT233522, MT233520, MT226610, MT198653, MT198651, MT198652, MT192773, MT192758, MT192772, MT192765, MT192759, MT188341, MT188340, MT188339, MT186676, MT186681, MT186677, MT186678, MT187977, MT186680, MT186682, MT186679, MT184909, MT184911, MT184912, MT184913, MT184910, MT184907, MT184908, CADDYA000000000, MT163718, MT163719, MT163720, MT163714, MT163715, MT163721, MT163717, MT163737, MT163738, MT163712, MT163716, MT159706, MT159716, MT159719, MT159707, MT159717, MT159709, MT159715, MT159718, MT159722, MT159708, MT161607, MT159705, MT159710, MT159711, MT159712, MT159713, MT159714, MT159720, MT159721, MT121215, MT159778, MT066156, LC529905, MT050493, MT012098, MT152900, MT152824, MT135044, MT135042, MT135041, MT135043, MT126808, MT127113, MT127114, MT127116, MT127115, LC528232, LC528233, MT123293, MT123291, MT123290, MT123292, MT118835, MT111896, MT111895, MT106052, MT106053, MT106054, MT093571, MT093631, MT081061, MT081063, MT081066, MT081062, MT081064, MT081065, MT081067, MT081059, MT081060, MT081068, MT072667, MT072668, MT072688, MT066157, MT066176, MT066159, MT066175, MT066158, LC523809, LC523807, LC523808, MT044258, MT044257, MT050416, MT050417, MT042773, MT042774, MT042775, MT042776, MT049951, MT050414, MT050415, MT042777, MT042778, MT039887, MT039888, MT039890, MT039873, LC522350, MT027062, MT027063, MT027064, MT020881, MT019530, MT019531, MT019533, MT020880, MT019532, MT019529, MT020781, LR757995, LR757998, LR757996, LR757997, MT007544, MT008022, MT008023, MN996531, MN996530, MN996527, MN996528, MN996529, MN997409, MN988668, MN988669, MN994467, MN994468, MN988713, MN938384, MN975262, MN985325, MN938386, MN938388, MN938385, MN938387, MN938390, MN938389, MN975263, MN975267, MN975268, MN975265, MN975264, MN975266, MN970004, MN970003, MN908947, each of which is incorporated herein by reference in its entirety.

In some embodiments, an antigenic sequence, antigen, and/or epitope is from a host subject cell. For example, antibodies that block viral entry can be generated by using an antigen or epitope from a component of a host cell that a virus uses as an entry factor.

An antigenic sequence, antigen, and/or epitope is from, for example, a bacteria, such as a bacterial surface protein, a bacterial membrane protein, a bacterial envelope protein, a bacterial inner membrane protein, a bacterial outer membrane protein, a bacterial periplasmic protein, a bacterial entry protein, a bacterial membrane fusion protein, a bacterial structural protein, a bacterial non-structural protein, a secreted bacterial protein, a bacterial polymerase protein, a bacterial DNA polymerase, a bacterial RNA polymerase, a bacterial protease, a bacterial glycoprotein, bacterial transcription factor, a bacterial enzyme, or a bacterial toxin.

In some embodiments, the antigenic sequence, antigen, and/or epitope is from one of these bacteria: Streptococcus agalactiae (also known as group B Streptococcus or GBS)); Streptococcus pyogenes (also known as group A Streptococcus (GAS)); Staphylococcus aureus; Methicillin-resistant Staphylococcus aureus (MRSA); Staphylococcus epidermis; Treponema pallidum; Francisella tularensis; Rickettsia; Yersinia pestis; Neisseria meningitidis: Antigens include, but are not limited to, membrane proteins such as adhesins, autotransporters, toxins, iron acquisition proteins, and factor H binding protein; Streptococcus pneumoniae; Moraxella catarrhalis; Bordetella pertussis: Antigens include, but are not limited to, pertussis toxin or toxoid (PT), filamentous haemagglutinin (FHA), pertactin, and agglutinogens 2 and 3; Clostridium tetani: the typical antigen is tetanus toxoid; Cornynebacterium diphtheriae: the typical antigen is diphtheria toxoid; Haemophilus influenzae; Pseudomonas aeruginosa; Chlamydia trachomatis; Chlamydia pneumoniae; Helicobacter pylori; Escherichia coli (Antigens include, but are not limited to, antigens derived from enterotoxigenic E. coli (ETEC), enteroaggregative E. coli (EAggEC), diffusely adhering E. coli (DAEC), enteropathogenic E. coli (EPEC), extraintestinal pathogenic E. coli (ExPEC) and/or enterohemorrhagic E. coli (EHEC)). ExPEC strains include uropathogenic E. coli (UPEC) and meningitis/sepsis-associated E. coli (MNEC). Also included are Bacillus anthracis; Clostridium perfringens or Clostridium botulinums; Legionella pneumophila; Coxiella burnetiid; Brucella, such as B. abortus, B. canis, B. melitensis, B. neotomae, B. ovis, B. suis, B. pinnipediae. Francisella, such as F. novicida, F. philomiragia, F. tularensis; Neisseria gonorrhoeae; Haemophilus ducreyi; Enterococcus faecalis or Enterococcus faecium; Staphylococcus saprophyticus; Yersinia enterocolitica; Mycobacterium tuberculosis; Listeria monocytogenes; Vibrio cholerae; Salmonella typhi; Borrelia burgdorferi; Porphyromonas gingivalis; Klebsiella.

An antigenic sequence, antigen, and/or epitope is from, for example, fungus, such as a fungal surface protein, a fungal membrane protein, a fungal envelope protein, a fungal inner membrane protein, a fungal outer membrane protein, a fungal periplasmic protein, a fungal entry protein, a fungal membrane fusion protein, a fungal structural protein, a fungal non-structural protein, a secreted fungal protein, a fungal polymerase protein, a fungal DNA polymerase, a fungal RNA polymerase, a fungal protease, a fungal glycoprotein, fungal transcription factor, a fungal enzyme, or a fungal toxin.

In some embodiments, the antigenic sequence, antigen, and/or epitope is from fungal antigens or antigenic sequences derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme; or from Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; the less common are Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

An antigenic sequence, antigen, and/or epitope is from, for example, a eukaryotic parasite surface protein, eukaryotic parasite membrane protein, a eukaryotic parasite envelope protein, a eukaryotic parasite entry protein, a eukaryotic parasite membrane fusion protein, a eukaryotic parasite structural protein, a eukaryotic parasite non-structural protein, a secreted eukaryotic parasite protein, a eukaryotic parasite polymerase protein, a eukaryotic parasite DNA polymerase, a eukaryotic parasite RNA polymerase, a eukaryotic parasite protease, a eukaryotic parasite glycoprotein, eukaryotic parasite transcription factor, a eukaryotic parasite enzyme, or a eukaryotic parasite toxin.

In some embodiments, the antigenic sequence, antigen, and/or epitope is from a parasite, such as from the Plasmodium genus, such as P. falciparum, P. vivax, P. malariae or P. ovale. In some embodiments, the antigen elicits an immune response against a parasite from the Caligidae family, particularly those from the Lepeophtheirus and Caligus genera e.g. sea lice such as Lepeophtheirus salmonis or Caligus rogercresseyi. In some embodiments, the antigen elicits an immune response against the parasite Toxoplasma gondii.

In some embodiments, the antigens and/or epitopes are cancer antigens (e.g., neoepitopes). For example, an antigen and/or epitope is a neoantigen and/or neoepitope that is associated with acute leukemia, astrocytomas, biliary cancer (cholangiocarcinoma), bone cancer, breast cancer, brain stem glioma, bronchioloalveolar cell lung cancer, cancer of the adrenal gland, cancer of the anal region, cancer of the bladder, cancer of the endocrine system, cancer of the esophagus, cancer of the head or neck, cancer of the kidney, cancer of the parathyroid gland, cancer of the penis, cancer of the pleural/peritoneal membranes, cancer of the salivary gland, cancer of the small intestine, cancer of the thyroid gland, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, cervical cancer, chronic leukemia, colon cancer, colorectal cancer, cutaneous melanoma, ependymoma, epidermoid tumors, Ewings sarcoma, gastric cancer, glioblastoma, glioblastoma multiforme, glioma, hematologic malignancies, hepatocellular (liver) carcinoma, hepatoma, Hodgkin's Disease, intraocular melanoma, Kaposi sarcoma, lung cancer, lymphomas, medulloblastoma, melanoma, meningioma, mesothelioma, multiple myeloma, muscle cancer, neoplasms of the central nervous system (CNS), neuronal cancer, small cell lung cancer, non-small cell lung cancer, osteosarcoma, ovarian cancer, pancreatic cancer, pediatric malignancies, pituitary adenoma, prostate cancer, rectal cancer, renal cell carcinoma, sarcoma of soft tissue, schwanoma, skin cancer, spinal axis tumors, squamous cell carcinomas, stomach cancer, synovial sarcoma, testicular cancer, uterine cancer, or tumors and their metastases, including refractory versions of any of the above cancers, or any combination thereof.

In some embodiments, the antigenic sequence, antigen, and/or epitope is from a tumor antigen selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcino embryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma); (e) prostate associated antigens such as PAP, PSA, PSMA, PSH-P1, PSM-P1, PSM-P2, associated with e.g., prostate cancer; (f) immunoglobulin idiotypes (associated with myeloma and B cell lymphomas, for example); (g) neoantigens. In certain embodiments, tumor antigens include, but are not limited to, pi 5, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, pl6, TAGE, PSCA, CT7, 43-9F, 5T4, 791 Tgp72, beta-HCG, BCA225, BTAA, CA 125, CA 15-3 (CA 27.29YBCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein cyclophilin C-associated protein), TAAL6, TAG72, TLP, TPS, and the like.

In some embodiments, the antigen and/or epitope is a toxin antigenic sequence, antigen and/or epitope, derived from, for example, toxin in a venom, such as a venom from a snake (e.g., most species of rattlesnakes (e.g., eastern diamondback rattlesnake), species of brown snakes (e.g., king brown snake and eastern brown snake), russel's viper, cobras (e.g., Indian cobra, king cobra), certain species of kraits (e.g., common krait), mambas (e.g., black mamba), saw-scaled viper, boomslang, dubois sea snake, species of taipans (e.g., coastal taipan and inland taipan snake), species of lanceheads (e.g., fer-de-lance and terciopelo), bushmasters, copperhead, cottonmouth, coral snakes, death adders, Belcher's sea snake, tiger snakes, Australian black snakes), spider (e.g., brown recluse, black widow spider, Brazilian wandering spider, funnel-web spider, button spider, Australian redback spider, katipo, false black widow, Chilean recluse spider, mouse spider, species of Macrothele, species of Sicarius, species of Hexpthalma, certain species of tarantulas), scorpion and other arachnids (e.g., fat-tailed scorpion, deathstalker scorpion, Indian red scorpion, species of Centruroides, species of Tityus such as the Brazilian yellow scorpion), insects (e.g., species of bees, species of wasps, certain ants such as fire ants, some species of lepidopteran caterpillars, certain species of centipede, remipede Xibalbanus tulumensis), fish (e.g., certain species of catfish (e.g., striped eel catfish and other eeltail catfishes), certain species of stingrays (e.g., blue-spotted stingray), lionfishes, stonefishes, scorpionfishes, toadfishes, rabbitfishes, goblinfishes, cockatoo waspfish, striped blenny, stargazers, chimaeras, weevers, dogfish sharks), cnidarians (e.g., certain species jellyfish (e.g., Irukanjdi jellyfish and box jellyfish), hydrozoans (e.g., Portuguese Man o'War), sea anemones, certain species of coral), a lizard (e.g., a gila monster, Mexican bearded lizard, certain species of Varanus (e.g., Komodo dragon), perentie, and lace monitor), a mammal (e.g., Southern short-tailed shrew, duck-billed platypus, European mole, Eurasian water shrew, Mediterranean water shrew, Northern short-tailed shrew, Elliot's short-tailed shrew, certain species of solenodon (e.g., Cuban solenodon, Hispaniolan solenodon), slow loins), mollusks (e.g., certain species of cone snail), cephalopods (e.g., certain species of octopus (e.g., blue-ringed octopus), squid, and cuttlefish), amphibians (e.g., frogs such as poison dart frogs, Bruno's casque-headed frog, Greening's frog, salamanders (e.g., Fire salamander, Iberian ribbed newt)

In some embodiments, the toxin is from a plant or fungi (e.g., mushrooms).

In some embodiments, the toxin antigen and/or epitope is derived from a toxin from a drug, such as digoxin.

In some embodiments, the toxin antigen and/or epitope is derived from a toxin such as a cyanotoxins, dinotoxins, myotoxins, cytotoxins (e.g., ricin, apitoxin, mycotoxins (e.g., aflatoxin), ochratoxin, citrinin, ergot alkaloid, patulin, fusarium, fumonisins, trichothecenes, cardiotoxin), tetrodotoxin, batrachotoxin, botulinum toxin A, tetanus toxin A, diptheria toxin, dioxin, muscarine, bufortoxin, sarin, hemotoxins, phototoxins, necrotoxins, nephrotoxins, and neurotoxins (e.g., calciseptine, cobrotoxin, calcicludine, fasciculin-I, calliotoxin).

Antigenic sequences, antigens, and/or epitopes from any number of microorganisms or cancers can be utilized in the circular polyribonucleotides. In some cases, the antigenic sequences, antigens, and/or epitopes are associated with or expressed by one microorganism disclosed above. In some embodiments, the antigenic sequences, antigens, and/or epitopes are associated with or expressed by two or more microorganisms disclosed above. In some cases, the antigenic sequences, antigens, and/or epitopes are associated with or expressed by one cancer disclosed above. In some embodiments, the antigenic sequences, antigens, and/or epitopes are associated with or expressed by two or more cancers disclosed above. In some embodiments, the antigens and/or epitopes are derived from toxins as disclosed above. In some embodiments, the antigens, and/or epitopes are from two or more toxins disclosed above.

The two or more microorganisms are related or unrelated. In some cases, two or more microorganisms are phylogenetically related. For example, the circular polyribonucleotides of the disclosure comprise or encode antigens and/or epitopes from two or more viruses, two or more members of a viral family, two or more members of a viral class, two or more members of a viral order, two or more members of a viral genus, two or more members of a viral species, two or more coronaviruses, two or more severe acute respiratory syndrome-associated viruses, two or more bacterial pathogens. In some embodiments, the two or more microorganisms are not phylogenetically related, for example, antigens and/or epitopes from a coronavirus and antigens and/or epitopes from Streptococcus pneumoniae can be utilized in a composition or method disclosed herein.

In some cases, two or more microorganisms are phenotypically related. For example, the circular polyribonucleotides of the disclosure comprise or encode antigens and/or epitopes from two or more respiratory pathogens, two or more select agents, two or more microorganisms associated with severe disease, two or more microorganisms associated with adverse outcomes in immunocompromised subjects, two or more microorganisms associated with adverse outcomes related to pregnancy, two or more microorganisms associated with hemorrhagic fever.

An antigenic sequence, antigen, and/or epitope of the disclosure may comprise a wild type sequence. When describing an antigenic sequence, antigen, and/or epitope, the term “wild type” refers to a sequence (e.g., a nucleic acid sequence or an amino acid sequence) that is naturally occurring and encoded by a genome (e.g., a viral genome). A species (e.g., microorganism species) can have one wild type sequence, or two or more wild type sequences (for example, with one canonical wild type sequence present in a reference microorganism genome, and additional variant wild type sequences present that have arisen from mutations).

When describing an antigenic sequence, antigen, and/or epitope, the terms “derivative” and “derived from” refers to a sequence (e.g., nucleic acid sequence or amino acid sequence) that differs from a wild type sequence by one or more nucleic acids or amino acids, for example, containing one or more nucleic acid or amino acid insertions, deletions, and/or substitutions relative to a wild type sequence.

An antigenic sequence, antigen, and/or epitope derivative sequence is a sequence that has at least 60%, 70%, 75%, 80%, 85%, 90%, 91%, 92%, 93%, 94%, 95%, 96%, 97%, 98%, 99%, or more sequence identity to a wild type sequence, for example, a wild type nucleic acid, protein, antigen, or epitope sequence.

“Sequence identity” and “sequence similarity” is determined by alignment of two peptide or two nucleotide sequences using global or local alignment algorithms. Sequences may then be referred to as “substantially identical” or “essentially similar” when they (when optimally aligned by for example the programs GAP or BESTFIT using default parameters) share at least a certain minimal percentage of sequence identity. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or EmbossWin version 2.10.0 (using the program “needle”). Alternatively or additionally, percent similarity or identity may be determined by searching against databases, using algorithms such as FASTA, BLAST, etc. Sequence identity refers to the sequence identity over the entire length of the sequence.

In some embodiments, an antigen or epitope contains one or more amino acid insertions, deletions, substitutions, or a combination thereof that affect the structure of an encoded protein. In some embodiments, an antigen or epitope contains one or more amino acid insertions, deletions, substitutions, or a combination thereof that affect the function of an encoded protein. In some embodiments, an antigen or epitope contains one or more amino acid insertions, deletions, substitutions, or a combination thereof that affect the expression or processing of an encoded protein by a cell.

In some embodiments, an antigenic sequence or epitope contains one or more nucleic acid insertions, deletions, substitutions, or a combination thereof that affect the structure of an encoded antigenic nucleic acid.

Amino acid insertions, deletions, substitutions, or a combination thereof can introduce a site for a post-translational modification (for example, introduce a glycosylation, ubiquitination, phosphorylation, nitrosylation, methylation, acetylation, amidation, hydroxylation, sulfation, or lipidation site, or a sequence that is targeted for cleavage). In some embodiments, amino acid insertions, deletions, substitutions, or a combination thereof remove a site for a post-translational modification (for example, remove a glycosylation, ubiquitination, phosphorylation, nitrosylation, methylation, acetylation, amidation, hydroxylation, sulfation, or lipidation site, or a sequence that is targeted for cleavage). In some embodiments, amino acid insertions, deletions, substitutions, or a combination thereof modify a site for a post-translational modification (for example, modify a site to alter the efficiency or characteristics of glycosylation, ubiquitination, phosphorylation, nitrosylation, methylation, acetylation, amidation, hydroxylation, sulfation, or lipidation site, or cleavage).

An amino acid substitution can be a conservative or a non-conservative substitution. A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide. A conservative amino acid change can be an amino acid change from one hydrophilic amino acid to another hydrophilic amino acid. Hydrophilic amino acids can include Thr (T), Ser (S), His (H), Glu (E), Asn (N), Gln (Q), Asp (D), Lys (K) and Arg (R). A conservative amino acid change can be an amino acid change from one hydrophobic amino acid to another hydrophilic amino acid. Hydrophobic amino acids can include Ile (I), Phe (F), Val (V), Leu (L), Trp (W), Met (M), Ala (A), Gly (G), Tyr (Y), and Pro (P). A conservative amino acid change can be an amino acid change from one acidic amino acid to another acidic amino acid. Acidic amino acids can include Glu (E) and Asp (D). A conservative amino acid change can be an amino acid change from one basic amino acid to another basic amino acid. Basic amino acids can include His (H), Arg (R) and Lys (K). A conservative amino acid change can be an amino acid change from one polar amino acid to another polar amino acid. Polar amino acids can include Asn (N), Gln (Q), Ser (S) and Thr (T). A conservative amino acid change can be an amino acid change from one nonpolar amino acid to another nonpolar amino acid. Nonpolar amino acids can include Leu (L), Val(V), Ile (I), Met (M), Gly (G) and Ala (A). A conservative amino acid change can be an amino acid change from one aromatic amino acid to another aromatic amino acid. Aromatic amino acids can include Phe (F), Tyr (Y) and Trp (W). A conservative amino acid change can be an amino acid change from one aliphatic amino acid to another aliphatic amino acid. Aliphatic amino acids can include Ala (A), Val (V), Leu (L) and Ile (I). In some embodiments, a conservative amino acid substitution is an amino acid change from one amino acid to another amino acid within one of the following groups: Group I: ala, pro, gly, gln, asn, ser, thr; Group II: cys, ser, tyr, thr; Group III: val, ile, leu, met, ala, phe; Group IV: lys, arg, his; Group V: phe, tyr, trp, his; and Group VI: asp, glu.

In some embodiments, an antigen derivative or epitope derivative of the disclosure comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, or at least 100 amino acid deletions relative to a sequence disclosed herein (e.g., a wild type sequence).

In some embodiments, an antigen derivative or epitope derivative of the disclosure comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to a sequence disclosed herein (e.g., a wild type sequence).

In some embodiments, an antigen derivative or epitope derivative of the disclosure comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to a sequence disclosed herein (e.g., a wild type sequence).

In some embodiments, an antigen derivative or epitope derivative of the disclosure comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40,10-15, 15-20, or 20-25 amino acid substitutions relative to a sequence disclosed herein (e.g., a wild type sequence).

In some embodiments, an antigen derivative or epitope derivative of the disclosure comprises 1, 2, 3, 4, 5, 6, 7, 8, 9, 10, 11, 12, 13, 14, 15, 16, 17, 18, 19, or 20 amino acid substitutions relative to a sequence disclosed herein (e.g., a wild type sequence).

The one or more amino acid substitutions can be at the N-terminus, the C-terminus, within the amino acid sequence, or a combination thereof The amino acid substitutions can be contiguous, non-contiguous, or a combination thereof.

In some embodiments, an antigen derivative or epitope derivative of the disclosure comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, at most 50, at most 60, at most 70, at most 80, at most 90, at most 100, at most 120, at most 140, at most 160, at most 180, or at most 200 amino acid deletions relative to a sequence disclosed herein (e.g., a wild type sequence).

In some embodiments, an antigen derivative or epitope derivative of the disclosure comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40, 10-15, 15-20, 20-25, 20-30, 30-50, 50-100, or 100-200 amino acid deletions relative to a sequence disclosed herein (e.g., a wild type sequence).

In some embodiments, an antigen derivative or epitope derivative of the disclosure comprises 1, 2, 3, 4, 5, 6, 7, 8, 9, 10, 11, 12, 13, 14, 15, 16, 17, 18, 19, or 20 amino acid deletions relative to a sequence disclosed herein (e.g., a wild type sequence).

The one or more amino acid deletions can be at the N-terminus, the C-terminus, within the amino acid sequence, or a combination thereof The amino acid deletions can be contiguous, non-contiguous, or a combination thereof.

In some embodiments, an antigen derivative or epitope derivative of the disclosure comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to a sequence disclosed herein (e.g., a wild type sequence).

In some embodiments, an antigen derivative or epitope derivative of the disclosure comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to a sequence disclosed herein (e.g., a wild type sequence).

In some embodiments, an antigen derivative or epitope derivative of the disclosure comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40,10-15, 15-20, or 20-25 amino acid insertions relative to a sequence disclosed herein (e.g., a wild type sequence).

In some embodiments, an antigen derivative or epitope derivative of the disclosure comprises 1, 2, 3, 4, 5, 6, 7, 8, 9, 10, 11, 12, 13, 14, 15, 16, 17, 18, 19, or 20 amino acid insertions relative to a sequence disclosed herein (e.g., a wild type sequence).

The one or more amino acid insertions can be at the N-terminus, the C-terminus, within the amino acid sequence, or a combination thereof The amino acid insertions can be contiguous, non-contiguous, or a combination thereof.

In some embodiments, the antigen or epitope is expressed by the circular polyribonucleotide. In some embodiments, the antigen or epitope is a product of rolling circle amplification of the circular polyribonucleotide.

The antigen or epitope may be produced in substantial amounts. As such, the antigen or epitope may be any proteinaceous molecule that can be produced. An antigen or epitope can be a polypeptide that can be secreted from a cell, or localized to the cytoplasm, nucleus or membrane compartment of a cell. In some embodiments, a polypeptide encoded by a circular polyribonucleotide of the disclosure comprises a fragment of an antigen disclosed herein. In some embodiments, a polypeptide encoded by a circular polyribonucleotide of the disclosure comprises a fusion protein comprising two or more antigens disclosed herein, or fragments thereof. In some embodiments, a polypeptide encoded by a circular polyribonucleotide of the disclosure comprises an epitope. In some embodiments, a polypeptide encoded by a circular polyribonucleotide of the disclosure comprises a fusion protein comprising two or more epitopes disclosed herein, for example, an artificial peptide sequence comprising a plurality of predicted epitopes from one or more microorganisms of the disclosure.

In some embodiments, exemplary antigens or epitopes that can be expressed from the circular polyribonucleotide disclosed herein include a secreted protein, for example, a protein (e.g., antigen and/or epitope) that naturally includes a signal peptide, or one that does not usually encode a signal peptide, but is modified to contain one. In some embodiments, an antigen or epitope that can be expressed from the circular polyribonucleotide is a membrane protein, for example, comprising a polypeptide sequence that is generally found as a membrane protein, or a polypeptide sequence that is modified to be a membrane protein. In some embodiments, exemplary proteins that can be expressed from the circular polyribonucleotide disclosed herein include an intracellular protein or cytosolic protein.

In some embodiments, the antigen has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, less than about 250 amino acids, less than about 200 amino acids, less than about 150 amino acids, less than about 140 amino acids, less than about 130 amino acids, less than about 120 amino acids, less than about 110 amino acids, less than about 100 amino acids, less than about 90 amino acids, less than about 80 amino acids, less than about 70 amino acids, less than about 60 amino acids, less than about 50 amino acids, less than about 40 amino acids, less than about 30 amino acids, less than about 25 amino acids, less than about 20 amino acids, less than about 15 amino acids, less than about 10 amino acids, less than about 5 amino acids, any amino acid length therebetween or less may be useful.

In some embodiments, the circular polyribonucleotide comprises one or more antigen sequences and is configured for persistent expression in a cell of a subject (e.g., a non-human animal having a humanized immune system of the disclosure) in vivo. In some embodiments, the circular polyribonucleotide is configured such that expression of the one or more expression sequences in the cell at a later time point is equal to or higher than an earlier time point. In such embodiments, the expression of the one or more antigen sequences can be either maintained at a relatively stable level or can increase over time. The expression of the antigen sequences can be relatively stable for an extended period of time. transiently or for only a limited amount of time, for example, at most 1, 2, 3, 4, 5, 6, 7, 8, 9, or 10 days.

In some embodiments, the circular polyribonucleotide expresses one or more antigens and/or epitopes in a non-human animal having a humanized immune system, e.g., transiently or long term. In certain embodiments, expression of the antigens and/or epitopes persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, expression of the antigens and/or epitopes persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or any time therebetween.

The antigen or epitope expression comprises translating at least a region of the circular polyribonucleotide provided herein. For example, a circular polyribonucleotide can be translated in a non-human animal having a humanized immune system to generate polypeptides that comprise one or more antigens and/or epitopes of the disclosure, thereby stimulating production of an adaptive immune response (e.g., antibody response and/or T cell response) in the non-human animal. In some embodiments, a circular polyribonucleotide of the disclosure is translated to produce one or more antigens and/or epitopes in a human subject, thereby stimulating production of an adaptive immune response (e.g., antibody response and/or T cell response) in a human subject.

In some embodiments, the methods for antigen or epitope expression comprises translation of at least 10%, at least 20%, at least 30%, at least 40%, at least 50%, at least 60%, at least 70%, at least 80%, at least 90%, or at least 95% of the total length of the circular polyribonucleotide into polypeptides. In some embodiments, the methods for antigen or epitope expression comprises translation of the circular polyribonucleotide into polypeptides of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids. In some embodiments, the methods for protein expression comprises translation of the circular polyribonucleotide into polypeptides of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, or about 1000 amino acids. In some embodiments, the methods comprise translation of the circular polyribonucleotide into continuous polypeptides as provided herein, discrete polypeptides as provided herein, or both.

In some embodiments, the methods for antigen or epitope expression comprise modification, folding, or other post-translation modification of the translation product. In some embodiments, the methods for antigen or epitope expression comprise post-translation modification in vivo, e.g., via cellular machinery.

### Diluent

In some embodiments, an immunogenic composition used in the methods of the disclosure comprises a circular polyribonucleotide and a diluent. In a particular embodiment, an immunogenic composition comprising a circular polyribonucleotide and a diluent is administered to a non-human animal having a humanized immune system for the production of human polyclonal antibodies.

A diluent is typically a non-carrier excipient. A non-carrier excipient serves as a vehicle or medium for a composition, such as a circular polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. A non-carrier excipient can be any inactive ingredient suitable for administration to a non-human animal, for example, suitable for veterinary use. Modification of compositions suitable for administration to humans in order to render the compositions suitable for administration to various animals is well understood, and the ordinarily skilled veterinary pharmacologist can design and/or perform such modification with merely ordinary, if any, experimentation.

In some embodiments, the circular polyribonucleotide may be delivered as a naked delivery formulation, such as comprising a diluent. A naked delivery formulation delivers a circular polyribonucleotide, to a cell without the aid of a carrier and without modification or partial or complete encapsulation of the circular polyribonucleotide, capped polyribonucleotide, or complex thereof.

A naked delivery formulation is a formulation that is free from a carrier and wherein the circular polyribonucleotide is without a covalent modification that binds a moiety that aids in delivery to a cell or without partial or complete encapsulation of the circular polyribonucleotide. In some embodiments, a circular polyribonucleotide without a covalent modification that binds a moiety that aids in delivery to a cell is a polyribonucleotide that is not covalently bound to a protein, small molecule, a particle, a polymer, or a biopolymer. A circular polyribonucleotide without covalent modification that binds a moiety that aids in delivery to a cell does contain a modified phosphate group. For example, a circular polyribonucleotide without a covalent modification that binds a moiety that aids in delivery to a cell does not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation is free of any or all of: transfection reagents, cationic carriers, carbohydrate carriers, nanoparticle carriers, or protein carriers. In certain embodiments, a naked delivery formulation is free from phtoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA),1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N—(N\N′-Dimethylaminoethane)-carbamoyl]Cholesterol Hydrochloride (DC-Cholesterol HC1), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

In some embodiments, a naked delivery formulation comprises a non-carrier excipient. In some embodiments, a non-carrier excipient comprises an inactive ingredient that does not exhibit a cell-penetrating effect. In some embodiments, a non-carrier excipient comprises a buffer, for example PBS. In some embodiments, a non-carrier excipient is a solvent, a non-aqueous solvent, a diluent, a suspension aid, a surface active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation comprises a diluent. A diluent may be a liquid diluent or a solid diluent. In some embodiments, a diluent is an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

### Carrier

In some embodiments, an immunogenic composition of the invention comprises a circular polyribonucleotide and a carrier.

In certain embodiments, an immunogenic composition comprises a circular polyribonucleotide as described herein in a vesicle or other membrane-based carrier. In certain embodiments, a carrier is a polymeric carrier (e.g., a polymeric carrier that is encapsulating or a polymeric carrier that is not encapsulating).

In other embodiments, an immunogenic composition of the invention comprises a circular polyribonucleotide in a cell, vesicle or other membrane-based carrier. In one embodiment, an immunogenic composition comprises a circular polyribonucleotide formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

In certain embodiments, an immunogenic composition of the invention comprises a circular polyribonucleotide and lipid nanoparticles. Lipid nanoparticles are another example of a carrier that provides a biocompatible and biodegradable delivery system for a circular polyribonucleotide molecule as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core—shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the circular polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N—(N\N′-Dimethylaminoethane)-carbamoyl]Cholesterol Hydrochloride (DC-Cholesterol HC1), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as a carrier or drug delivery vehicles for a circular polyribonucleotide molecule described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a circular polyribonucleotide molecule described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a circular polyribonucleotide molecule described herein.

Virosomes and virus-like particles (VLPs) can also be used as carriers to deliver a circular polyribonucleotide molecule described herein to targeted cells.

In some embodiments, a pharmaceutical formulation disclosed herein comprises: (i) a compound (e.g., circular polyribonucleotide or antibody) disclosed herein; (ii) a buffer; (iii) a non-ionic detergent; (iv) a tonicity agent; and (v) a stabilizer. In some embodiments, the pharmaceutical formulation disclosed herein is a stable liquid pharmaceutical formulation.

### Adjuvant

An adjuvant is administered to a non-human animal having a humanized immune system for the production of human polyclonal antibodies from a circular polyribonucleotide as disclosed herein. In some embodiments, an adjuvant and circular polyribonucleotide are co-administered to a non-human animal having a humanized immune system in separate compositions. In some embodiments, an adjuvant is mixed or formulated with a circular polyribonucleotide in a single composition to obtain an immunogenic composition that is administered to a non-human animal having a humanized immune system.

Adjuvants may be a TH1 adjuvant and/or a TH2 adjuvant. Preferred adjuvants include, but are not limited to, one or more of the following:

Mineral-containing compositions. Mineral-containing compositions suitable for use as adjuvants in the invention include mineral salts, such as aluminum salts, and calcium salts. The invention includes mineral salts such as hydroxides (e.g. oxyhydroxides), phosphates (e.g. hydroxyphoshpates, orthophosphates), sulphates, etc., or mixtures of different mineral compounds, with the compounds taking any suitable form (e.g. gel, crystalline, amorphous, etc.). Calcium salts include calcium phosphate (e.g., the “CAP”). Aluminum salts include hydroxides, phosphates, sulfates, and the like.

Oil emulsion compositions. Oil-emulsion compositions suitable for use as adjuvants in the invention include squalene-water emulsions, such as MF59 (5% Squalene, 0.5% Tween 80 and 0.5% Span, formulated into submicron particles using a microfluidizer), AS03 (α-tocopherol, squalene and polysorbate 80 in an oil-in-water emulsion), Montanide formulations (e.g. Montanide ISA 51, Montanide ISA 720), incomplete Freunds adjuvant (IFA), complete Freund's adjuvant (CFA), and incomplete Freund's adjuvant (IFA).

Small molecules. Small molecules suitable for use as adjuvants in the invention include imiquimod or 847, resiquimod or R848, or gardiquimod.

Polymeric nanoparticles. Polymeric nanoparticles suitable for use as an adjuvant in the invention include poly(a-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

Saponin (i.e., a glycoside, polycyclic aglycones attached to one or more sugar side chains). Saponin formulations suitable for use as an adjuvant in the invention include purified formulations, such as QS21, as well as lipid formulations, such as ISCOMs and ISCOMs matrix. QS21 is marketed as STIMULON(™). Saponin formulations may also comprise a sterol, such as cholesterol. Combinations of saponins and cholesterols can be used to form unique particles called immunostimulating complexes (ISCOMs). ISCOMs typically also include a phospholipid such as phosphatidylethanolamine or phosphatidylcholine. Any known saponin can be used in ISCOMs. Preferably, the ISCOM includes one or more of QuilA, QHA & QHC. Optionally, the ISCOMS may be devoid of additional detergent.

Lipopolysaccharides. Adjuvants suitable for use in the invention include non-toxic derivatives of enterobacterial lipopolysaccharide (LPS). Such derivatives include monophosphoryl lipid A (MPLA), glucopyranosyl lipid A (GLA) and 3-O-deacylated MPL (3dMPL). 3dMPL is a mixture of 3 De-O-acylated monophosphoryl lipid A with 4, 5 or 6 acylated chains. Other non-toxic LPS derivatives include monophosphoryl lipid A mimics, such as aminoalkyl glucosaminide phosphate derivatives e.g. RC-529.

Liposomes. Liposomes suitable for use as an adjuvant in the invention include virosomes and CAF01.

Lipid nanoparticles. Adjuvants suitable for use in the invention include lipid nanoparticles and their components

Lipopeptides (i.e., compounds comprising one or more fatty acid residues and two or more amino acid residues). Lipopeptide suitable for use as an adjuvant in the invention include Pam2 (Pam2CSK4) and Pam3 (Pam3CSK4).

Glycolipids. Glycolipids suitable for use as an adjuvant in the invention include cord factor (trehalose dimycolate).

Peptides and peptidoglycans derived from (synthetic or purified) gram negative or gram positive bacteria, such as MDP (N-acetyl-muramyl-L-alanyl-D-isoglutamine) are suitable for use as an adjuvant in the invention

Carbohydrates (carbohydrate containing) or polysaccharides suitable for use as an adjuvant include dextran (e.g., branched microbial polysaccharide), dextran-sulfate, lentinan, zymosan, beta-glucan, deltin, mannan, and chitin.

RNA based adjuvants. RNA based adjuvants suitable for use in the invention include poly IC, poly IC:LC, hairpin RNAs with or without a 5′triphosphate, viral sequences, polyU containing sequence, dsRNA natural or synthetic RNA sequences, and nucleic acid analogs (e.g., cyclic GMP-AMP or other cyclic dinucleotides e.g., cyclic di-GMP, immunostimulatory base analogs e.g., C8-substituted and N7,C8-disubstituted guanine ribonucleotides).

DNA based adjuvants. DNA based adjuvants suitable for use in the invention include CpGs, dsDNA, and natural or synthetic immunostimulatory DNA sequences.

Proteins or peptides. Proteins and peptides suitable for use as an adjuvant in the invention include flagellin-fusion proteins, MBL (mannose-binding lectin), cytokines, and chemokines.

Viral particles. Viral particles suitable for use as an adjuvant include virosomes (phospholipid cell membrane bilayer).

An adjuvant for use in the invention may be bacterial derived, such as a flagellin, LPS, or a bacterial toxin (e.g., enterotoxins (protein), e.g., heat-labile toxin or cholera toxin). An adjuvant for use in the invention may be a hybrid molecule such as CpG conjugated to imiquimod. An adjuvant for use in the invention may be a fungal or oomycete MAMPs, such as chitin or beta-glucan. In some embodiments, an adjuvant is an inorganic nanoparticle, such as gold nanorods or silica based nanoparticles (e.g., mesoporous silica nanoparticles (MSN)). In some embodiments, an adjuvant is a multi-component adjuvant or adjuvant system, such as AS01, AS03, AS04 (MLP5+alum), CFA (complete Freund's adjuvant: IFA+peptiglycan+trehalose dimycolate), CAF01 (two component system of cationic liposome vehicle (dimethyl dioctadecyl-ammonium (DDA)) stabilized with a glycolipid immunomodulator (trehalose 6,6-dibehenate (TDB), which can be a synthetic variant of cord factor located in the mycobacterial cell wall).

Cytokines. An adjuvant may be a partial or full-length DNA encoding a cytokine such as, a pro-inflammatory cytokine (e.g., GM-CSF, IL-1 alpha, IL-1 beta, TGF-beta, TNF-alpha, TNF-beta), Th-1 inducing cytokines (e.g., IFN-gamma, IL-2, IL-12, IL-15, IL-18), or Th-2 inducing cytokines (e.g., IL-4, IL-5, IL-6, IL-10, IL-13).

Chemokines. An adjuvant may be a partial or full-length DNA encoding a chemokine such as, MCP-1, MIP-1 alpha, MIP-1 beta, Rantes, or TCA-3.

An adjuvant may be a partial or full-length DNA encoding a costimulatory molecule, such as CD80, CD86, CD40-L, CD70, or CD27.

An adjuvant may be a partial or full length DNA encoding for an innate immune sentinel (partial, full-length, or mutated) or a constitutively active (ca) innate immune sentinel, such as caTLR4, casting, caTLR3, caTLR3, caTLR9, caTLR7, caTLR8, caTLR7, caRIG-I/DDX58, or caMDA-5/IFIH1.

An adjuvant may be a partial or full length DNA encoding for an adaptor or signaling molecule, such as STING, TRIF, TRAM, MyD88, IPS1, ASC, MAVS, MAPKs, IKK-alpha, IKK complex, TBK1, beta-catenin, and caspase 1.

An adjuvant may be a partial or full length DNA encoding for a transcriptional activator, such as a transcription activator that can upregulate an immune response (e.g., AP1, NF-kappa B, IRF3, IRF7, IRF1, or IRF5). An adjuvant may be a partial or full length DNA encoding for a cytokine receptor, such as IL-2beta, IFN-gamma, or IL-6.

An adjuvant may be a partial or full length DNA encoding for a bacterial component, such as flagellin or MBL.

An adjuvant may be a partial or full length DNA encoding for any component of the innate immune system.

In a particular embodiment, an adjuvant used in the invention is a SAB's proprietary adjuvant formulation, SAB-adj-1 or SAB-adj-2.

### Vaccine

In methods described herein, the non-human animal may also be administered a second vaccine. In some embodiments, a composition that is administered to a non-human animal having a humanized immune system comprises a circular polyribonucleotide and a vaccine. In some embodiments, a vaccine and circular polyribonucleotide are co-administered in separate compositions. The vaccine is simultaneously administered with the circular polyribonucleotide immunization, administered before the circular polyribonucleotide immunization, or after the circular polyribonucleotide immunization.

For example, in some embodiments, a non-human animal having a humanized immune system is immunized with a non-circular polyribonucleotide coronavirus vaccine (e.g., protein subunit vaccine) and an immunogenic composition comprising a circular polyribonucleotide. In some embodiments, a non-human animal having a humanized immune system is immunized with a non-polyribonucleotide vaccine for a first microorganism (e.g., pneumococcus) and an immunogenic composition comprising a circular polyribonucleotide that comprises or encodes antigens and/or antigens for a second microorganism (e.g., a coronavirus). A vaccine can be any bacterial infection vaccine or viral infection vaccine. In a particular embodiment, a vaccine is a pneumococcal polysaccharide vaccine, such as PCV13 or PPSV23. In some embodiments, the vaccine is an influenza vaccine. In some embodiments, the vaccine is an RSV vaccine (e.g., palivizumap).

### Non-Human Animal Having a Humanized Immune System

A non-human animal having a humanized immune system is used to produce human polyclonal antibodies from a circular polyribonucleotide as disclosed herein. In certain embodiments, a non-human animal having a humanized immune system is a mammal. A non-human animal includes an ungulate, for example, a donkey, a goat, a horse, a cow, or a pig. A non-human animal having a humanized immune system includes a rabbit, a mouse, a rat, or a chicken. In a particular embodiment, the non-human animal is a cow (bovine). In another embodiment, the non-human animal is a goat (caprine). In another particular embodiment, the non-human animal is a rat. In another embodiment, the non-human animal is a rabbit.

In some embodiments, a non-human animal with a humanized immune system is used for producing hyperimmune plasma, e.g., plasma with a high concentration of antibodies that bind to antigenic sequences, antigens, and/or epitopes of circular polyribonucleotide as disclosed herein.

A non-human animal having a humanized immune system is an animal that produces human antibodies, or antibody variants, fragments, and derivatives thereof A humanized immune system comprises a humanized immunoglobulin gene locus, or multiple humanized immunoglobulin gene loci.

In some embodiments, humanized immunoglobulin gene locus comprises a germ line sequence of human immunoglobulin, allowing the non-human animal to produce humanized antibodies (e.g., fully human antibodies).

A non-human animal having a humanized immune system comprises non-human B cells with a humanized immunoglobulin gene locus. The humanized immunoglobulin gene locus can undergo VDJ recombination during B cell development, thereby allowing for generation of B cells with great diversity of antigen binging specificity.

The binding specificity of antibodies are generated by the process of VDJ recombination. The exons encoding the antigen binding portions (the variable regions) are assembled by chromosomal breakage and rejoining in developing B cells. The exons encoding the antigen binding domains are assembled from so-called V (variable), D (diversity), and J (joining) gene segments by “cut and paste” DNA rearrangements. This process, termed V(D)J recombination, chooses a pair of segments, introduces double-strand breaks adjacent to each segment, deletes (or, in selected cases, inverts) the intervening DNA, and ligates the segments together. Rearrangements occur in an ordered fashion, with D to J joining proceeding before a V segment is joined to the rearranged DJ segments. This process of combinatorial assembly—choosing one segment of each type from several (sometimes many) possibilities is the fundamental engine driving antigen receptor diversity in mammals. Diversity is tremendously amplified by the characteristic variability at the junctions (loss or gain of small numbers of nucleotides) between the various segments. This process leverages a relatively small investment in germline coding capacity into an almost limitless repertoire of potential antigen binding specificities.

A non-human animal having a humanized immune system comprises a plurality of B cells of diverse specificities generated by VDJ recombination, for example, of the humanized immunoglobulin gene locus. A B cell that encodes a B cell receptor (and an antibody) that specifically binds to an antigen and/or epitope of the disclosure is activated upon countering cognate antigen, for example, after encountering the antigen and/or epitope that is expressed from a circular polyribonucleotide of the disclosure. The activated B cell produces antibodies that specifically bind the antigen and/or epitope of the disclosure. The activated B cell can proliferate. In some embodiments, the activated non-human B cell differentiates into memory B cells and/or plasma cells. In some embodiments, the activated non-human B cell undergoes class switching to generate antibodies of different isotypes as disclosed herein. In some embodiments, the non-human B cell undergoes somatic hypermutation to generate antibodies that bind to an antigen and/or epitope with higher affinity.

Upon immunization with one or more immunogenic compositions comprising one or more circular polyribonucleotides that comprise an antigenic sequence or express an antigen and/or multiple epitopes, a plurality of B cell clones respond to their respective cognate epitopes, leading to the production of polyclonal antibodies with a plurality of binding specificities. In some embodiments, immunizing a non-human animal having a humanized immune system with one or more immunogenic compositions comprising one or more circular polyribonucleotides activates at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 non-human B cell clones. In some embodiments, immunizing a non-human animal having a humanized immune system with one or more immunogenic compositions comprising one or more circular polyribonucleotides leads to production of polyclonal antiserum that comprises antibodies that specifically bind at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 antigens and/or epitopes.

Various techniques for modifying the genome of non-human animals can be employed to develop an animal capable of producing human polyclonal antibodies. A non-human animal can be a transgenic animal, for example, a transgenic animal comprising all or a substantial portion of the humanized immunoglobulin gene locus or loci. A non-human animal can be a transchromosomal animal, for example, a non-human animal that comprises a human artificial chromosome or a yeast artificial chromosome.

A humanized immunoglobulin gene locus can be present on a vector, for example, a human artificial chromosome or a yeast artificial chromosome (YAC). A human artificial chromosome (HAC) comprising the humanized immunoglobulin gene locus can be introduced into an animal. A vector (e.g., HAC) can contain the germline repertoire of the human antibody heavy chain genes (from human chromosome 14) and the human antibody light chain genes, for example, one or both of kappa (from human chromosome 2) and lambda (from human chromosome 22). The HAC can be transferred into cells of the non-human animal species and the transgenic animals can be produced by somatic cell nuclear transfer.

In some embodiments, a humanized immunoglobulin gene locus is integrated into the non-human animal's genome. For example, techniques comprising homologous recombination or homology-directed repair can be employed to modify the animal's genome to introduce the human nucleotide sequences. Tools such as CRISPR/Cas, TALEN, and zinc finger nucleases can be used to target integration.

Methods of generating non-human animals with humanized immune systems have been disclosed. For example, a human artificial chromosome is generated and transferred into a cell that comprises additional genomic modifications of interest (e.g., deletions of endogenous non-human immune system genes), and the cell is used as a nuclear donor to generate a transgenic non-human animal.

In some embodiments, the humanized immune system comprises one or more human antibody heavy chains, wherein each gene encoding an antibody heavy chain is operably linked to a class switch regulatory element. Operably linked can mean that a first DNA molecule (e.g., heavy chain gene) is joined to a second DNA molecule (e.g., class switch regulatory element), wherein the first and second DNA molecules are arranged so that the first DNA molecule affects the function of the second DNA molecule. The two DNA molecules may or may not be part of a single contiguous DNA molecule and may or may not be adjacent. For example, a promoter is operably linked to a transcribable DNA molecule if the promoter is capable of affecting the transcription or translation of the transcribable DNA molecule.

In some embodiments, the humanized immune system comprises one or more human antibody light chains. In some embodiments, the humanized immune system comprises one or more human antibody surrogate light chains.

In some embodiments, the humanized immune system comprises an amino acid sequence that is derived from the non-human animal, for example, a constant region, such as a heavy chain constant region or a part thereof. In some embodiments, a humanized immune system comprises an IgM heavy chain constant region from the non-human animal (for example, an ungulate-derived IgM heavy chain constant region). In some embodiments, at least one class switch regulatory element of the genes encoding the one or more human antibody heavy chains is replaced with a non-human (e.g., ungulate-derived) class switch regulatory element, for example, to allow antibody class switching when antibodies are raised against antigens and/or epitopes of the disclosure within the non-human animal.

A humanized immunoglobulin gene locus can comprise non-human elements that are incorporated for compatibility with the non-human animal. In some embodiments, a non-human element is present in a humanized immunoglobulin gene locus to reduce recognition by any remaining elements of the non-human animal's immune system). In some embodiments, an immunoglobulin gene (e.g., IgM) is partly replaced with an amino acid sequence from the non-human animal. In some embodiments, a non-human regulatory element is present in a humanized immunoglobulin gene locus to facilitate expression and regulation of the locus within the non-human animal.

A humanized immunoglobulin gene locus can comprise a human DNA sequence. A humanized immunoglobulin gene locus can be codon optimized to facilitate expression of the encompassed genes (e.g., antibody genes) in the non-human animal.

A non-human animal having a humanized immune system can comprise or can lack endogenous non-human immune system components. In some embodiments, a non-human animal having a humanized immune system lacks non-humanized antibodies (e.g., lack the ability to produce non-humanized antibodies). In a particular embodiment, a non-human animal having a humanized immune system lacks, for example, one or more non-human immunoglobulin heavy chain genes, one or more non-human immunoglobulin light chain genes, or a combination thereof.

A non-human animal having a humanized immune system can retain, for example, non-human immune cells. A non-human animal having a humanized immune system can retain non-human innate immune system components (e.g., cells, complement, antimicrobial peptides, etc.). In some embodiments, a non-human animal with a humanized immune system retains non-human T cells. In some embodiments, a non-human animal having a humanized immune system retains non-human B cells. In some embodiments, a non-human animal having a humanized immune system retains non-human antigen-presenting cells. In some embodiments, a non-human animal having a humanized immune system retains non-human antibodies.

In some embodiments, a humanized immune system comprises human innate immune proteins, for example, complement proteins.

In some embodiments, a humanized immune system comprises humanized T cells and/or antigen-presenting cells.

In some embodiments, compositions and methods of the disclosure comprise T cells. For example, a circular polyribonucleotide of the disclosure comprises or encode for both antigens recognized by B cells and T cells, and upon immunization of a non-human animal with a humanized immune system, the T cells provide T cell help, thereby increasing antibody production in the non-human animal.

In some embodiments, the non-human animal having a humanized immune system comprises any feature or any combination of features or any methods of making as disclosed in US20170233459, which is hereby incorporated by reference in its entirety. In some embodiments, the non-human animal having a humanized immune system comprises any feature or any combination of features or any methods of making as disclosed in Kuroiwa, Y et al. Nat Biotechnol, 2009 Feb; 27(2):173-81; Matsushita, H. et al. PLos ONE, 2014 Mar. 6; 9(3): e90383; Hooper, J. W. et al. Sci Transl Med, 2014 Nov. 26; 6(264): 264ra162; Matsushit, H. et al., PLoS ONE 2015 Jun. 24; 10(6): e0130699; Luke, T. et al. Sci Transl Med, 2016 Feb. 17; 8(326): 326ra21; Dye, J. et al., Sci Rep. 2016 Apr. 25; 6:24897; Gardner, C. et al. J Virol. 2017 Jun. 26; 91(14); Stein, D. et al., Antiviral Res 2017 October; 146:164-173; Silver, J. N., Clin Infect Dis. 2018 Mar. 19; 66(7):1116-1119; Beigel, J. H. et al., Lancet Infect Dis, 2018 April; 18;(4):410-418; Luke, T. et al., J Inf Dis. 2018 Nov. 33; 218(suppl_5):S636-S648; Wu et al. Sci Rep 2019 Jan. 23; (9), 366; Lee et al. Nat Biotechnol; 2014 Mar. 16; (32): 356-363; and Kazuki et al. PNAS, 2019 February, 116 (8): 3072-3081, each of which is hereby incorporated by reference in its entirety.

### Methods of Producing Polyclonal Antibodies

Human polyclonal antibodies are produced by administering a composition comprising a circular polyribonucleotides as described herein to a non-human animal having a humanized immune system. In a particular embodiment, a non-human animal having a humanized immune system is immunized with a circular polyribonucleotide to stimulate the adaptive immune response and production of polyclonal antibodies (e.g., human polyclonal antibodies) that bind to desired antigens and/or epitopes of the circular polyribonucleotide or expressed from the circular polyribonucleotide. The antigen comprises one or more epitopes for producing the polyclonal antibodies. In some embodiments, the non-human animal having a humanized immune system is further administered an adjuvant. In some embodiments the non-human animal having a humanized immune system is further immunized with a vaccine. After immunization with the circular polyribonucleotide, the produced human polyclonal antibodies are purified from the non-human animal having a humanized immune system. The human polyclonal antibodies generated are used as a treatment or prophylactic. The human polyclonal antibodies provide protection against or treatment for, for example, a microorganism that expresses the antigens and/or epitopes, a cancer that expresses the antigens and/or epitopes, or a toxin.

### Immunization

In some embodiments, methods of producing human polyclonal antibodies comprise immunizing a non-human animal having a humanized immune system with a circular polyribonucleotide as described above. The circular polyribonucleotide comprises an antigenic sequence (e.g., due to its secondary structure or tertiary structure) that stimulates the production of human polyclonal antibodies in the non-human animal having a humanized immune system. In some embodiments, an antigen and/or epitope is expressed from the circular polyribonucleotide that stimulates the production of human polyclonal antibodies in the non-human animal having a humanized immune system. In some embodiments, the circular polyribonucleotide both comprises an antigenic sequence itself and expresses an antigen and/or epitope for producing human polyclonal antibodies in a non-human animal having a humanized immune system. In some embodiments, the non-human animal comprises a humanized immunoglobulin gene locus and a circular polyribonucleotide comprising an antigenic sequence and/or a sequence encoding an antigen and/or epitope. In some embodiments, the non-human animal having a humanized immune system is further administered an adjuvant, such as any adjuvant described herein. In some embodiments, the non-human animal having a humanized immune system is further immunized with a vaccine, such as any vaccine describe herein. The immunizations are carried out as described above.

In some embodiments, the method further comprises pre-administering an agent to improve immunogenic responses to the non-human animal having a humanized immune system. In some embodiments, the agent is the antigen as disclosed herein (e.g., a protein antigen). For example, the method comprises administering the protein antigen from 1 to 7 days prior to administration of the circular polyribonucleotide comprising the sequence encoding the protein antigen. In some embodiments, the protein antigen is administered 1, 2, 3, 4, 5, 6, or 7 days prior to administration of the circular polyribonucleotide comprising the sequence encoding the protein antigen. The protein antigen may be administered as a protein preparation, encoded in a plasmid (pDNA), presented in a virus-like particle (VLP), formulated in a lipid nanoparticle, or the like.

In some embodiments, the method further comprises evaluating the non-human animal for antibody response to the antigen. In some embodiments, the evaluating is before and/or after administration of the circular polyribonucleotide.

### Plasma Collection

Plasma comprising human polyclonal antibodies produced from a circular polyribonucleotide as disclosed herein is collected from a non-human animal having a humanized immune system that was immunized with the circular polyribonucleotide. These human polyclonal antibodies are used in a prophylactic or treatment of a disease associated with an antigen and/or epitope of the circular polyribonucleotide or expressed from the circular polyribonucleotide. Plasma is collected via plasmapheresis. Plasma is collected from the same non-human animal having a humanized immune system once or multiple times, for example, multiple times each during a given period of time after an immunization, multiple times after an immunization, multiple times in between immunizations, or any combination thereof.

Plasma is collected from a non-human animal having a humanized immune system any suitable amount of time following an immunization, for example the first immunization, the most recent immunization, or an intermediate immunization. Plasma is collected from the non-human animal having a humanized immune system at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, after an immunization. In some embodiments, plasma is collected from the non-human animal having a humanized immune system at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most at most 35, at most 42, at most 49, or at most 56 days after an immunization. In some embodiments, plasma is collected from the non-human animal having a humanized immune system about 1, 2, 3, 4, 5, 6, 7, 8, 9, 10, 11, 12, 14, 16, 17, 18, 20, 21, 22, 23, 24, 25, 26, 27, 28, 29, 30, 31, 32, 33, 34, 35, 36, 37, 38, 39, 40, 41, or 42 days, or more after an immunization.

Plasma can be frozen (e.g., stored or transported frozen). In some embodiments, plasma is maintained fresh, or antibodies are purified from fresh plasma.

In some embodiments, a composition comprises the collected plasma. In a particular embodiment, a composition comprises plasma from a non-human animal comprising a humanized immune system and a circular polyribonucleotide comprising a sequence encoding an antigen. In some embodiments, a composition comprises plasma from a non-human animal comprising a humanized immune system and a circular polyribonucleotide comprising a sequence encoding an antigen, and the antigen. In some embodiments, a composition comprises plasma from a non-human animal comprising a humanized immune system and a circular polyribonucleotide comprising an antigenic sequence.

### Human Polyclonal Antibody Purification

Human polyclonal antibodies produced from a circular polyribonucleotide as disclosed herein is purified after plasma collection from a non-human animal having a humanized immune system that is immunized with the circular polyribonucleotide. These human polyclonal antibodies are used in a prophylactic or treatment of a disease associated with an antigen and/or epitope of the circular polyribonucleotide or expressed from the circular polyribonucleotide. In certain embodiments, the human polyclonal antibodies are used as an antivenom. In some embodiments, the polyclonal antibodies are administered to a human subject in need thereof.

Human polyclonal antibodies produced using the methods of the disclosure are purified from plasma. For example, plasma is pH-adjusted to 4.8 (e.g., with dropwise addition of 20% acetic acid), fractionated by caprylic acid at a caprylic acid/total protein ratio of 1.0, and then clarified by centrifugation (e.g., at 10,000 g for 20 min at room temperature). The supernatant containing polyclonal antibodies (e.g., IgG polyclonal antibodies) is neutralized to pH 7.5 with 1 M tris, 0.22 μm filtered, and affinity-purified with an anti-human immunoglobulin-specific column (e.g., anti-human IgG light chain-specific column). The polyclonal antibodies are further purified by passage over an affinity column that specifically binds impurities, for example, non-human antibodies from the non-human animal. The polyclonal antibodies are stored in a suitable buffer, for example, a sterile-filtered buffer consisting of 10 mM glutamic acid monosodium salt, 262 mM D-sorbitol, and Tween (0.05 mg/ml) (pH 5.5). The quantity and concentration of the purified polyclonal antibodies are determined. HPLC size exclusion chromatography can be conducted to determine whether aggregates or multimers are present.

In some embodiments, the human polyclonal antibodies are purified from a non-human animal having a humanized immune system according to Beigel, J H et al. (Lancet Infect Dis., 18:410-418 (2018), including Supplementary appendix)), which is herein incorporated by reference in its entirety. Briefly, human IgG polyclonal antibodies from a non-human animal having a humanized immune system are purified using chromatography. Fully human IgG is separated from the non-human animal IgG using a human IgG kappa chain specific affinity column (e.g., KappaSelect from GE healthcare) as a capture step. The human IgG kappa chain specific affinity column specifically binds the fully human IgG with minimum cross-reactivity to non-human animal IgG Fc and IgG. Further non-human animal IgG is removed using an IgG Fc specific affinity column that binds to the specifically binds to the non-human animal IgG (e.g., for bovine, Capto HC15 from GE healthcare), which is used as a negative affinity step to specifically clear the non-human animal IgG. An anion exchange chromatography step is also used to further reduce contaminants, such as host DNA, endotoxin, IgG aggregates and leached affinity ligands.

### Polyclonal Antibodies

The human polyclonal antibodies of the disclosure are produced by methods that utilize circular polyribonucleotides and a non-human animal with a humanized immune system as disclosed herein. In a particular embodiment, circular polyribonucleotides that comprise an antigenic sequence or encode antigens and/or epitopes are administered (e.g., via an injection) to a non-human animal with a humanized immune system, thereby stimulating production of human polyclonal antibodies that bind to the antigenic sequence or the antigens and/or epitopes expressed by the circular polyribonucleotide. These human polyclonal antibodies bind to antigens and/or epitopes of interest (e.g., viral antigens and/or epitopes, such as coronavirus antigens and/or epitopes) and are useful as a treatment or prophylactic. In a particular embodiment, these human polyclonal antibodies provide protection (e.g., passive immunization) against a microorganism that expresses the antigens and/or epitopes. In some embodiments, the human polyclonal antibodies are collected from plasma after immunization of the non-human animal having a humanized immune system with a circular polyribonucleotide. In some embodiments, the collected plasma is purified for the human polyclonal antibodies. Furthermore, the human polyclonal antibodies are formulated for administration to a human subject, for example, as a treatment or prophylactic. In some embodiments, the human polyclonal antibodies provide protection against a microorganism that expresses the antigens and/or epitopes. In some embodiments, the human polyclonal antibodies provide protection against a cancer that expresses the antigens and/or epitopes. In some embodiments, the human polyclonal antibodies provide protection against a toxin comprising the antigens and/or epitopes. In some embodiments, the human polyclonal antibodies are used as an antivenom.

Human polyclonal antibodies of the disclosure bind to an antigenic sequence, antigen, and/or epitope. The antigen or epitope can be the same antigen or epitope of a circular polyribonucleotide or expressed from a circular polyribonucleotide disclosed herein. In some embodiments, the antigen or epitope is any antigen or epitope disclosed herein (e.g., an antigen or epitope from a microorganism, e.g., from a coronavirus).

The human polyclonal antibodies bind to any number of antigenic sequences, antigens, and/or epitopes disclosed herein. In some embodiments, human polyclonal antibodies bind to, for example, at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more antigenic sequences, antigens, and/or epitopes.

In some embodiments, human polyclonal antibodies bind to, for example, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less antigenic sequences, antigens, and/or epitopes.

In some embodiments, human polyclonal antibodies bind to, for example, about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 antigenic sequences, antigens, and/or epitopes.

In a particular embodiment, human polyclonal antibodies bind to, for example, antigens and/or epitopes from at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, or more microorganisms, cancer antigens, toxin antigens, or a combination thereof.

In some embodiments, human polyclonal antibodies bind to antigens and/or epitopes from at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, or less microorganisms, cancer antigens, toxin antigens, or a combination thereof.

In some embodiments, human polyclonal antibodies bind to antigens and/or epitopes from about 1, 2, 3, 4, 5, 6, 7, 8, 9, 10, 15, 20, 25, 30, 40, 50, 60, 70, 80, 90, or 100, microorganisms, cancer antigens, toxin antigens, or a combination thereof.

Human polyclonal antibodies bind to one or more epitopes from an antigen or antigenic sequence. In some embodiments, an antigen comprises a nucleic acid sequence or amino acid sequence, which contains multiple epitopes (e.g., epitopes recognized by B cells and/or T cells) therein, and antibody clones bind to one or more of those epitopes.

Human polyclonal antibodies of the disclosure bind to, for example, at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more epitopes from one antigen.

In some embodiments, human polyclonal antibodies bind to, for example, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, or at most 500, or less epitopes from one antigen.

In some embodiments, polyclonal antibodies bind to, for example, about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 epitopes from one antigen.

A composition of this disclosure comprises the human polyclonal antibodies as described herein. A composition of this disclosure comprises the human polyclonal antibodies in collected plasma from a non-human animal having a humanized immune system as described herein. A composition of this disclosure comprises the purified human polyclonal antibodies as described herein.

Human polyclonal antibodies of the disclosure bind to variants of an antigen or epitope. Variants can be naturally-occurring variants (for example, variants identified in sequence data from different viral genera, species, isolates, or quasispecies), or can be derivative sequences as disclosed herein that have been generated in silico (for example, antigen or epitopes with one or more amino acid insertions, deletions, substitutions, or a combination thereof compared to a wild type antigen or epitope).

In some embodiments, human polyclonal antibodies bind to, for example, at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more variants of an antigenic sequence, antigen, and/or epitope.

In some embodiments, polyclonal antibodies bind to, for example, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less variants of an antigenic sequence, antigen, and/or epitope.

In some embodiments, polyclonal antibodies bind to, for example, about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 variants of an antigenic sequence, antigen, and/or epitope.

Human polyclonal antibodies of the disclosure are neutralizing antibodies, non-neutralizing antibodies, or a combination thereof.

Human polyclonal antibodies of the disclosure are antibodies that comprise the basic four chain antibody unit. The basic four chain antibody unit can comprise two heavy chain (H) polypeptide sequences and two light chain (L) polypeptide sequences. Each of the heavy chains can comprise one N-terminal variable (V_{H}) region and three or four C-terminal constant (C_{H}1, C_{H}2, C_{H}3, and C_{H}4) regions. Each of the light chains can comprise one N-terminal variable (V_{L}) region and one C-terminal constant (C_{L}) region. The light chain variable region is aligned with the heavy chain variable region and the light chain constant region is aligned with first heavy chain constant region C_{H1}. The pairing of a heavy chain variable region and light chain variable region together forms a single antigen-binding site. Each light chain is linked to a heavy chain by one covalent disulfide bond. The two heavy chains are linked to each other by one or more disulfide bonds depending on the heavy chain isotype. Each heavy and light chain can also comprise regularly-spaced intrachain disulfide bridges. The C-terminal constant regions of the heavy chains comprise the Fc region of the antibody, which can mediate effector functions, for example, through interactions with Fc receptors or complement proteins.

The light chain can be designated kappa or lambda based on the amino acid sequence of the constant region. The heavy chain can be designated alpha, delta, epsilon, gamma, or mu based on the amino acid sequence of the constant region. Antibodies are categorized into five immunoglobulin classes, or isotypes, based on the heavy chain. IgA comprises alpha heavy chains, IgD comprises delta heavy chains, IgE comprises epsilon heavy chains, IgG comprises gamma heavy chains, and IgM comprises mu heavy chains. Antibodies of the IgG, IgD, and IgE classes comprise monomers of the four chain unit described above (two heavy and two light chains), while the IgM and IgA classes can comprise multimers of the four chain unit. The alpha and gamma classes are further divided into subclasses on the basis of differences in the sequence and function of the heavy chain constant region. Subclasses of IgA and IgG expressed by humans include IgG1, IgG2, IgG3, IgG4, IgA1 and IgA2.

Illustrative amino acid sequences of human constant domain sequences are provided in TABLE 2. In some embodiments, an antibody, non-human animal, or non-human B cell comprises a human IgG1 constant domain sequence, for example, comprises SEQ ID NO: 11, or a variant, derivative, or fragment thereof. In some embodiments, an antibody, non-human animal, or non-human B cell comprises a human IgG2 constant domain sequence, for example, comprises SEQ ID NO: 12 or a variant, derivative, or fragment thereof. In some embodiments, an antibody, non-human animal, or non-human B cell comprises a human IgG3 constant domain sequence, for example, comprises SEQ ID NO: 13 or a variant, derivative, or fragment thereof. In some embodiments, an antibody, non-human animal, or non-human B cell comprises a human IgG4 constant domain sequence, for example, comprises SEQ ID NO: 14 or a variant, derivative, or fragment thereof. In some embodiments, an antibody, non-human animal, or non-human B cell comprises a human IgE constant domain sequence, for example, comprises SEQ ID NO: 15 or a variant, derivative, or fragment thereof. In some embodiments, an antibody, non-human animal, or non-human B cell comprises a human IgAl constant domain sequence, for example, comprises SEQ ID NO: 16 or a variant, derivative, or fragment thereof. In some embodiments, an antibody, non-human animal, or non-human B cell comprises a human IgA2 constant domain sequence, for example, comprises SEQ ID NO: 17 or a variant, derivative, or fragment thereof. In some embodiments, an antibody, non-human animal, or non-human B cell comprises a human IgM constant domain sequence, for example, comprises SEQ ID NO: 18 or a variant, derivative, or fragment thereof. In some embodiments, an antibody, non-human animal, or non-human B cell comprises a human IgD constant domain sequence, for example, comprises SEQ ID NO: 19 or a variant, derivative, or fragment thereof.

TABLE 2
Illustrative amino acid sequences of human
constant domain sequences.
TABLE 2

| SEQ | | |
|---|---|---|
| ID | | |
| NO: | Name | Amino acid sequence |
| 11 | IgG1 | ASTKGPSVFPLAPSSKSTSGGTAALGCLVKDYFPE |
| | constant | PVTVSWNSGALTSGVHTFPAVLQSSGLYSLSSVVT |
| | | VPSSSLGTQTYICNVNHKPSNTKVDKKVEPKSCDK |
| | | THTCPPCPAPELLGGPSVFLFPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LPAPIEKTISKAKGQPREPQVYTLPPSRDELTKNQ |
| | | VSLTCLVKGFYPSDIAVEWESNGQPENNYKTTPPV |
| | | LDSDGSFFLYSKLTVDKSRWQQGNVFSCSVMHEAL |
| | | HNHYTQKSLSLSPGK |
| 12 | IgG2 | ASTKGPSVFPLAPCSRSTSESTAALGCLVKDYFPE |
| | constant | PVTVSWNSGALTSGVHTFPAVLQSSGLYSLSSVVT |
| | | VPSSNFGTQTYTCNVDHKPSNTKVDKTVERKCCVE |
| | | CPPCPAPPVAGPSVFLFPPKPKDTLMISRTPEVTC |
| | | VVVDVSHEDPEVQFNWYVDGVEVHNAKTKPREEQF |
| | | NSTFRVVSVLTVVHQDWLNGKEYKCKVSNKGLPAP |
| | | IIEKTSKTKGQPREPQVYTLPPSREEMTKNQVSLT |
| | | CLVKGFYPSDISVEWESNGQPENNYKTTPPMLDSD |
| | | GSFFLYSKLTVDKSRWQQGNVFSCSVMHEALHNHY |
| | | TQKSLSLSPGK |
| 13 | IgG3 | ASTKGPSVFPLAPCSRSTSGGTAALGCLVKDYFPE |
| | constant | PVTVSWNSGALTSGVHTFPAVLQSSGLYSLSSVVT |
| | | VPSSSLGTQTYTCNVNHKPSNTKVDKRVELKTPLG |
| | | DTTHTCPRCPEPKSCDTPPPCPRCPEPKSCDTPPP |
| | | CPRCPEPKSCDTPPPCPRCPAPELLGGPSVFLFPP |
| | | KPKDTLMISRTPEVTCVVVDVSHEDPEVQFKWYVD |
| | | GVEVHNAKTKPREEQYNSTFRVVSVLTVLHQDWLN |
| | | GKEYKCKVSNKALPAPIEKTISKTKGQPREPQVYT |
| | | LPPSREEMTKNQVSLTCLVKGFYPSDIAVEWESSG |
| | | QPENNYNTTPPMLDSDGSFFLYSKLTVDKSRWQQG |
| | | NIFSCSVMHEALHNRFTQKSLSLSPGK |
| 14 | IgG4 | ASTKGPSVFPLAPCSRSTSESTAALGCLVKDYFPE |
| | constant | PVTVSWNSGALTSGVHTFPAVLQSSGLYSLSSVVT |
| | | VPSSSLGTKTYTCNVDHKPSNTKVDKRVESKYGPP |
| | | CPPCPAPEFLGGPSVFLFPPKPKDTLMISRTPEVT |
| | | CVVVDVSQEDPEVQFNWYVDGVEVHNAKTKPREEQ |
| | | FNSTYRVVSVLTVLHQDWLNGKEYKCKVSNKGLPS |
| | | SIEKTISKAKGQPREPQVYTLPPSQEEMTKNQVSL |
| | | TCLVKGFYPSDIAVEWESNGQPENNYKTTPPVLDS |
| | | DGSFFLYSRLTVDKSRWQEGNVFSCSVMHEALHNH |
| | | YTQKSLSLSLGK |
| 15 | IgE | ASTQSPSVFPLTRCCKNIPSNATSVTLGCLATGYF |
| | constant | PEPVMVTWDTGSLNGTTMTLPATTLTLSGHYATIS |
| | | LLTVSGAWAKQMFTCRVAHTPSSTDWVDNKTFSVC |
| | | SRDFTPPTVKILQSSCDGGGHFPPTIQLLCLVSGY |
| | | TPGTINITWLEDGQVMDVDLSTASTTQEGELASTQ |
| | | SELTLSQKHWLSDRTYTCQVTYQGHTFEDSTKKCA |
| | | DSNPRGVSAYLSRPSPFDLFIRKSPTITCLVVDLA |
| | | PSKGTVNLTWSRASGKPVNHSTRKEEKQRNGTLTV |
| | | TSTLPVGTRDWIEGETYQCRVTHPHLPRALMRSTT |
| | | KTSGPRAAPEVYAFATPEWPGSRDKRTLACLIQNF |
| | | MPEDISVQWLHNEVQLPDARHSTTQPRKTKGSGFF |
| | | VFSRLEVTRAEWEQKDEFICRAVHEAASPSQTVQR |
| | | AVSVNPGK |
| 16 | IgA1 | ASPTSPKVFPLSLCSTQPDGNVVIACLVQGFFPQE |
| | constant | PLSVTWSESGQGVTARNFPPSQDASGDLYTTSSQL |
| | | TLPATQCLAGKSVTCHVKHYTNPSQDVTVPCPVPS |
| | | TPPTPSPSTPPTPSPSCCHPRLSLHRPALEDLLLG |
| | | SEANLTCTLTGLRDASGVTFTWTPSSGKSAVQGPP |
| | | ERDLCGCYSVSSVLPGCAEPWNHGKTFTCTAAYPE |
| | | SKTPLTATLSKSGNTFRPEVHLLPPPSEELALNEL |
| | | VTLTCLARGFSPKDVLVRWLQGSQELPREKYLTWA |
| | | RQEPSQGTTTFAVTSILRVAAEDWKKGDTFSCMVG |
| | | HEALPLAFTQKTISDRLAGKPTHVNVSVVMAEVDG |
| | | TCY |
| 17 | IgGA2 | ASPTSPKVFPLSLDSTPQDGNVVVACLVQGFFPQE |
| | constant | PLSVTWSESGQNVTARNFPPSQDASGDLYTTSSQL |
| | | TLPATQCPDGKSVTCHVKHYTNSSQDVTVPCRVPP |
| | | PPPCCHPRLSLHRPALEDLLLGSEANLTCTLTGLR |
| | | DASGATFTWTPSSGKSAVQGPPERDLCGCYSVSSV |
| | | LPGCAQPWNHGETFTCTAAHPELKTPLTANITKSG |
| | | NTFRPEVHLLPPPSEELALNELVTLTCLARGFSPK |
| | | DVLVRWLQGSQELPREKYLTWASRQEPSQGTTTYA |
| | | VTSILRVAAEDWKKGETFSCMVGHEALPLAFTQKT |
| | | IDRMAGKPTHINVSVVMAEADGTCY |
| 18 | IgM | GSASAPTLFPLVSCENSPSDTSSVAVGCLAQDFLP |
| | constant | DSITFSWKYKNNSDISSTRGFPSVLRGGKYAATSQ |
| | | VLLPSKDVMQGTDEHVVCKVQHPNGNKEKNVPLPV |
| | | IAELPPKVSVFVPPRDGFFGNPRKSKLICQATGFS |
| | | PRQIQVSWLREGKQVGSGVTTDQVQAEAKESGPTT |
| | | YKVTSTLTIKESDWLGQSMFTCRVDHRGLTFQQNA |
| | | SSMCVPDQDTAIRVFAIPPSFASIFLTKSTKLTCL |
| | | VTDLTTYDSVTISWTRQNGEAVKTHTNISESHPNA |
| | | TFSAVGEASICEDDWNSGERFTCTVTHTDLPSPLK |
| | | QTISRPKGVALHRPDVYLLPPAREQLNLRESATIT |
| | | CLVTGFSPADVFVQWMQRGQPLSPEKYVTSAPMPE |
| | | PQAPGRYFAHSILTVSEEEWNTGETYTCVVAHEAL |
| | | PNRVTERTVDKSTGKPTLYNVSLVMSDTAGTCY |
| 19 | IgD | APTKAPDVFPIISGCRHPKDNSPVVLACLITGYHP |
| | constant | TSVTVTWYMGTQSQPQRTFPEIQRRDSYYMTSSQL |
| | | STPLQQWRQGEYKCVVQHTASKSKKEIFRWPESPK |
| | | AQASSVPTAQPQAEGSLAKATTAPATTRNTGRGGE |
| | | EKKKEKEKEEQEERETKTPECPSHTQPLGVYLLTP |
| | | AVQDLWLRDKATFTCFVVGSDLKDAHLTWEVAGKV |
| | | PTGGVEEGLLERHSNGSQSQHSRLTLPRSLWNAGT |
| | | SVTCTLNHPSLPPQRLMALREPAAQAPVKLSLNLL |
| | | ASSDPPEAASWLLCEVSGFSPPNILLMWLEDQREV |
| | | NTSGFAPARPPPQPRSTTFWAWSVLRVPAPPSPQP |
| | | ATYTCVVSHEDSRTLLNASRSLEVSYVTDHGPMK |

An antibody of the disclosure can comprise a human light chain constant domain sequence, e.g. a kappa (IgK) or lambda (IgL) chain. In some embodiments, an antibody, non-human animal, or non-human B cell comprises a human IgK constant domain sequence, for example, comprises SEQ ID NO: 20 or a variant, derivative, or fragment thereof. In some embodiments, an antibody, non-human animal, or non-human B cell comprises a human IgL constant domain sequence, for example, comprises SEQ ID NO: 21 or a variant, derivative, or fragment thereof.

TABLE 3
TABLE 3 provides example light chain
constant domain sequences.

| SEQ | | |
|---|---|---|
| ID | | |
| NO: | Name | Amino acid sequence |
| 20 | IgK | TVAAPSVFIFPPSDEQLKSGTASVVCLLNNFYPR |
| | constant | EAKVQWKVDNALQSGNSQESVTEQDSKDSTYSLS |
| | | STLTLSKADYEKHKVYACEVTHQGLSSPVTKSFN |
| | | RGEC |
| 21 | IgL | GQPKANPTVTLFPPSSEELQANKATLVCLISDFY |
| | constant | PGAVTVAWKADGSPVKAGVETTKPSKQSNNKYAA |
| | | SSYLSLTPEQWKSHRSYSCQVTHEGSTVEKTVAP |
| | | TECS |

The constant regions can mediate various effector functions and can be minimally involved in antigen binding. Different IgG isotypes or subclasses can be associated with different effector functions or therapeutic characteristics, for example, because of interactions with different Fc receptors and/or complement proteins. Antibodies comprising Fc regions that engage activating Fc receptors can, for example, participate in antibody-dependent cell-mediated cytotoxicity (ADCC), antibody-dependent cellular phagocytosis (ADCP), complement-dependent cytotoxicity (CDC), induction of signaling through immunoreceptor tyrosine-based activation motifs (ITAMs), and induction of cytokine secretion. Antibodies comprising Fc regions that engage inhibitory Fc receptors can, for example, induce signaling through immunoreceptor tyrosine-based inhibitory motifs (ITIMs).

Different antibody subclasses comprise different abilities to elicit immune effector functions. For example, IgG1 and IgG3 can effectively recruit complement to activate CDC, IgG2 elicits minimal ADCC. IgG4 has a lesser ability to trigger immune effector functions. Modifications to the constant regions can also affect antibody characteristics, for example, enhancement or reduction of Fc receptor ligation, enhancement or reduction of ADCC, enhancement or reduction of ADCP, enhancement or reduction of CDC, enhancement or reduction of signaling through ITAMs, enhancement or reduction of cytokine induction, enhancement or reduction of signaling through ITIMs, enhancement or reduction of half-life, or enhancement or reduction of co-engagement of antigen with Fc receptors. Modifications can include, for example, amino acid mutations, altering post-translational modifications (e.g., glycosylation), combining domains from different isotypes or subclasses, or a combination thereof.

Antibodies of the disclosure can comprise constant regions or Fc regions that are selected or modified to provide suitable antibody characteristics, for example, suitable characteristics for treating a disease or condition as disclosed herein. In some embodiments, IgG1 can be used, for example, to promote inflammation, immune activation, and immune effector functions for the treatment of an infection. In some embodiments, IgG4 can be used, for example, in cases where antagonistic properties of the antibody with reduced immune effector functions are desired (e.g., to neutralize viral antigens and inhibit viral entry into cells without promoting inflammation and immune activation).

Non-limiting examples of antibody modifications and their effects are provided in TABLE 4.

TABLE 4

| Effect | Isotype | Mutation(s)/modification(s) |
|---|---|---|
| Enhanced ADCC | IgG1 | F243L/R292P/Y300L/V305I/ |
| | | P396L |
| Enhanced ADCC | IgG1 | S239D/I332E |
| Enhanced ADCC | IgG1 | S239D/I332E/A330L |
| Enhanced ADCC | IgG1 | S298A/E333A/K334A |
| Enhanced ADCC | IgG1 | In one heavy chain: L234Y/ |
| | | L235Q/G236W/S239M/H268D/ |
| | | D270E/S298A |
| | | In the opposing heavy chain: |
| | | D270E/K326D/A330M/K334E |
| Enhanced ADCP | IgG1 | G236A/S239D/I332E |
| Enhanced CDC | IgG1 | K326W/E333S |
| Enhanced CDC | IgG1 | S267E/H268F/S324T |
| Enhanced CDC | IgG1, | Combination of domains from |
| | IgG3 | IgG1/IgG3 |
| Enhanced CDC | IgG1 | E345R/E430G/S440Y |
| Loss of glycosylation, | IgG1 | N297A or N297Q or N297G |
| reduced effector | | |
| functions | | |
| Reduced effector functions | IgG1, | L235E |
| | IgG4 | |
| Reduced effector functions | IgG1 | L234A/L235A |
| Reduced effector functions | IgG4 | F234A/L235A |
| Reduced effector functions | IgG4 | F234A/L235A/G237A/P238S |
| Reduced effector functions | IgG4 | F234A/L235A/ΔG236/G237A/ |
| | | P238S |
| Reduced effector functions | IgG2, | Combination of domains from |
| | IgG4 | IgG2/IgG4 |
| Reduced effector functions | IgG2 | H268Q/V309L/A330S/P331S |
| Reduced effector functions | IgG2 | V234A/G237A/P238S/H268A/ |
| | | V309L/A330S/P331S |
| Reduced effector functions | IgG1 | L234A/L235A/G237A/P238S/ |
| | | H268A/A330S/P331S |
| Increased half-life | IgG1 | M252Y/S254T/T256E |
| Increased half-life | IgG1 | M428L/N434S |
| Increased antigen/Fc | IgG1 | S267E/L328F |
| receptor coengagement | | |
| Altered antigen/Fc | IgG1 | N325S/L328F |
| receptor coengagement | | |
| Reduced Fab arm exchange | IgG4 | S228P |

The variable (V) regions mediate antigen binding and define the specificity of a particular antibody for an antigen. The variable region comprises relatively invariant sequences called framework regions, and hypervariable regions, which differ considerably in sequence among antibodies of different binding specificities. The variable region of each antibody heavy or light chain comprises four framework regions separated by three hypervariable regions. The variable regions of heavy and light chains fold in a manner that brings the hypervariable regions together in close proximity to create an antigen binding site. The four framework regions largely adopt an f3-sheet configuration, while the three hypervariable regions form loops connecting, and in some cases forming part of, the f3-sheet structure.

Within hypervariable regions are amino acid residues that primarily determine the binding specificity of the antibody. Sequences comprising these residues are known as complementarity determining regions (CDRs). One antigen binding site of an antibody can comprise six CDRs, three in the hypervariable regions of the light chain, and three in the hypervariable regions of the heavy chain. The CDRs in the light chain can be designated LCDR1, LCDR2, LCDR3, while the CDRs in the heavy chain can be designated HCDR1, HCDR2, and HCDR3.

In some embodiments, antibodies of the disclosure include variants, derivatives, and antigen-binding fragments thereof. For example, a non-human animal can be genetically modified to produce antibody variants, derivatives, and antigen-binding fragments thereof. In some embodiments, an antibody can be a single domain antibody (sdAb), for example, a heavy chain only antibody (HCAb) VHH, or nanobody. Non-limiting examples of antigen-binding fragments include Fab, Fab′, F(ab′)₂, dimers and trimers of Fab conjugates, Fv, scFv, minibodies, dia-, tria-, and tetrabodies, and linear antibodies. Fab and Fab′ are antigen-binding fragments that can comprise the V_{H} and C_{H}1 domains of the heavy chain linked to the V_{L} and C_{L} domains of the light chain via a disulfide bond. A F(ab′)₂ can comprise two Fab or Fab′ that are joined by disulfide bonds. A Fv can comprise the V_{H} and V_{L} domains held together by non-covalent interactions. A scFv (single-chain variable fragment) is a fusion protein that can comprise the V_{H} and V_{L} domains connected by a peptide linker. Manipulation of the orientation of the V_{H} and V_{L} domains and the linker length can be used to create different forms of molecules that can be monomeric, dimeric (diabody), trimeric (triabody), or tetrameric (tetrabody). Minibodies are scFv-C_{H}3fusion proteins that assemble into bivalent dimers.

### Pharmaceutical Compositions

Human polyclonal antibodies, or variants, fragments, and derivatives thereof are antibodies that are formulated for administration to a human. In some embodiments, the human polyclonal antibodies are collected and purified as described herein from a non-human animal having a humanized immune system, and then formulated in a pharmaceutical composition. In some embodiments, pharmaceutical compositions provided herein are suitable for administration to humans.

In some embodiments, the pharmaceutical composition comprises a pharmaceutically acceptable excipient. A pharmaceutically acceptable excipient can be a non-carrier excipient. A non-carrier excipient serves as a vehicle or medium for a composition, such as a circular polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

Pharmaceutical compositions can be sterile and stable under the conditions of manufacture and storage. The composition can be formulated as a solution, microemulsion, liposome, or other ordered structure suitable to high drug concentration. Examples of suitable aqueous and non-aqueous compositions which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Sterile injectable solutions can be prepared by incorporating the active compound (e.g., human polyclonal antibodies) in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The pharmaceutical compositions of the disclosure can be prepared in a composition that will protect them against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978. A composition of the disclosure can be, for example, an immediate release form or a controlled release formulation. An immediate release formulation can be formulated to allow the compounds (e.g., human polyclonal antibodies) to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

Pharmaceutical formulations for administration can include aqueous solutions of the active compounds (e.g., human polyclonal antibodies) in water soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the agents to allow for the preparation of highly concentrated solutions. The active ingredient can be in powder form for constitution with a suitable vehicle, for example, sterile pyrogen-free water, before use.

Methods for the preparation of compositions comprising the agents described herein include formulating the agents with one or more inert, pharmaceutically-acceptable excipients or carriers to form a solid, semi-solid, or liquid composition. Solid compositions include, for example, powders, dispersible granules, and cachets. Liquid compositions include, for example, solutions in which an agent is dissolved, emulsions comprising an agent, or a solution containing liposomes, micelles, or nanoparticles comprising an agent as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

In some embodiments, a formulation of the disclosure contains a thermal stabilizer, such as a sugar or sugar alcohol, for example, sucrose, sorbitol, glycerol, trehalose, or mannitol, or any combination thereof. In some embodiments, the stabilizer is a sugar. In some embodiments, the sugar is sucrose, mannitol or trehalose.

Pharmaceutical compositions as described herein can be formulated for example to include a pharmaceutical excipient or carrier. A pharmaceutical carrier may be a membrane, lipid bilayer, and/or a polymeric carrier, e.g., a liposome or particle such as a nanoparticle, e.g., a lipid nanoparticle, and delivered by known methods to a subject in need thereof (e.g., a human or non-human agricultural or domestic animal, e.g., cattle, dog, cat, horse, poultry). Such methods include, but not limited to, transfection (e.g., lipid-mediated, cationic polymers, calcium phosphate); electroporation or other methods of membrane disruption (e.g., nucleofection), fusion, and viral delivery (e.g., lentivirus, retrovirus, adenovirus, AAV).

The invention is further directed to a host or host cell comprising the circular polyribonucleotide as described herein. In some embodiments, vertebrate, mammal (e.g., human), or other organism or cell.

In some embodiments, a host or a host cell is contacted with (e.g., delivered to or administered to) the circular polyribonucleotide. In some embodiments, the host is a mammal, such as an ungulate. The amount of the circular polyribonucleotide, expression product, or both in the host can be measured at any time after administration.

### Therapeutic Methods

The disclosure provides compositions and methods that are useful as treatments or prophylactics, for example, compositions and methods that comprise human polyclonal antibodies that can be used to protect a subject against the effects of an infection. The human polyclonal antibodies provide protection against, for example, a microorganism that expresses the antigens and/or epitopes. In some embodiments, the disclosure provides compositions for use in treating or prophylaxis of an infection.

Non-limiting examples of conditions and diseases that can be treated by compositions and methods of the disclosure include those caused by or associated with a microorganism disclosed herein, for example infections. In some embodiments, a condition is caused by or associated with a virus of the disclosure. In some embodiments, a condition is caused by or associated with a bacterium of the disclosure. In some embodiments, a condition is caused by or associated with a fungus of the disclosure. In some embodiments, a condition is caused by or associated with a eukaryotic parasite of the disclosure. In some embodiments, a condition is caused by or associated with a coronavirus of the disclosure. In some embodiments, a condition is caused by or associated with a SARS-CoV. In some embodiments, a condition is caused by or associated with SARS-CoV-2. In some embodiments, a condition is coronavirus disease of 2019 (COVID-19). In some embodiments, a condition is caused by or associated with MERS-CoV.

Non-limiting examples of conditions and diseases that can be treated by compositions and methods of the disclosure include those caused by or associated with a cancer disclosed herein, for example a cancer expressing HER2 or a neoantigen.

Non-limiting examples of conditions that can be treated by compositions and methods of the disclosure include those caused by or associated with a toxin disclosed herein, for example toxicity caused by a bite or sting from a venomous animal, absorption of a toxin, inhalation of a toxin, ingestion of a toxin, or a drug overdose.

In some embodiments, human polyclonal antibodies are administered to a human subject. In some embodiments, the human polyclonal antibodies are formulated in a human polyclonal antibody preparation. A method of producing a human polyclonal antibody preparation against a target (e.g. an antigen of a pathogen, cancer, toxin) comprising (a) administering to a non-human animal capable of producing human antibodies an immunogenic composition comprising a circular polyribonucleotide that comprises a sequence encoding an antigen of the target, (b) collecting blood or plasma from the non-human animal, (c) purifying human polyclonal antibodies against the target from the blood or plasma, and (d) formulating human polyclonal antibodies as a therapeutic or pharmaceutical preparation for human use.

In practicing the methods of treatment or use provided herein, therapeutically-effective amounts of the compounds (e.g., human polyclonal antibodies) described herein are administered in pharmaceutical compositions to a human subject having a disease or condition to be treated or requiring prophylaxis. A therapeutically-effective amount can vary widely depending on the severity of the disease, the age and relative health of the human subject, the potency of the compounds used, the microorganism, and other factors.

### Methods and Routes of Administering

The human polyclonal antibodies disclosed herein (e.g., in a pharmaceutical composition of the polyclonal antibodies) are administered in a therapeutically-effective amount by various forms and routes including, for example, oral, or topical administration. In some embodiments, the polyclonal antibodies or a pharmaceutical composition thereof are administered by parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, the polyclonal antibodies or a pharmaceutical composition thereof are administered by absorption through epithelial or mucocutaneous linings (e.g., oral mucosa, rectal and intestinal mucosa administration). In some embodiments, the polyclonal antibodies or a pharmaceutical composition thereof is delivered via multiple administration routes.

In some embodiments, the human polyclonal antibodies or a pharmaceutical composition thereof is administered by intravenous infusion. In some embodiments, the human polyclonal antibodies or a pharmaceutical composition thereof is administered by slow continuous infusion over a long period, such as more than 24 hours. In some embodiments, the human polyclonal antibodies or a pharmaceutical composition thereof is administered as an intravenous injection or a short infusion.

The human polyclonal antibodies or a pharmaceutical composition thereof are administered in a local manner, for example, via injection of the polyclonal antibodies directly into an organ, optionally in a depot or sustained release formulation or implant. The human polyclonal antibodies or a pharmaceutical composition thereof are provided in the form of a rapid release formulation, in the form of an extended release formulation, or in the form of an intermediate release formulation. A rapid release form can provide an immediate release. An extended release formulation provides a controlled release or a sustained delayed release. In some embodiments, a pump is used for delivery of the human polyclonal antibodies or a pharmaceutical composition thereof. In some embodiments, a pen delivery device is used, for example, for subcutaneous delivery of the polyclonal antibodies or a pharmaceutical composition thereof of the disclosure.

The human polyclonal antibodies or a pharmaceutical composition thereof provided herein are administered in conjunction with other therapies, for example, an antiviral therapy, an antibiotic, a cell therapy, a cytokine therapy, or an anti-inflammatory agent. In some embodiments, human antibody described herein is used singly or in combination with one or more therapeutic agents as a component of mixtures. In some embodiments, the human polyclonal antibodies or a pharmaceutical composition thereof are combined with a treatment for an additional treatment the subject may be in need thereof For example, the human polyclonal antibodies that bind to a coronavirus antigen are administered with a treatment for a pneumococcal infection to a subject with a coronavirus infection and a pneumococcal infection or risk of pneumococcal infection.

### Doses and Frequency

The human polyclonal antibodies or a pharmaceutical composition thereof described herein are administered before, during, or after the occurrence of a disease or condition, and the timing of administering the human polyclonal antibodies or pharmaceutical composition thereof can vary. In some cases, the human polyclonal antibodies or a pharmaceutical composition thereof are used as a prophylactic and administered continuously to subjects with a susceptibility to an infection or a propensity to a condition or disease associated with an infection. Prophylactic administration can lessen a likelihood of the occurrence of the infection, disease or condition, or can reduce the severity of the infection, disease or condition. In some embodiments, administering occurs before a subject is exposed to a disease or during a subject's exposure to a disease. For example, administering the human polyclonal antibodies or a pharmaceutical composition thereof to a health care worker before working with patients having a disease or while working with patients having a disease.

The human polyclonal antibodies or a pharmaceutical composition thereof are administered to a subject after (e.g., as soon as possible after) the onset of the symptoms. The human polyclonal antibodies or a pharmaceutical composition thereof are administered to a subject after (e.g., as soon as possible after) a test result, for example, a test result that provides a diagnosis, a test that shows the presence of a microorganism in a subject, or a test showing progress of a condition, e.g., a decreased blood oxygen levels. The human polyclonal antibodies or a pharmaceutical composition thereof are administered after (e.g., as soon as is practicable after) the onset of a disease or condition is detected or suspected. The human polyclonal antibodies or a pharmaceutical composition thereof are administered after (e.g., as soon as is practicable after) a potential exposure to a microorganism, for example, after a subject has contact with an infected subject, or learns they had contact with an infected subject that may be contagious.

The human polyclonal antibodies or a pharmaceutical composition thereof described herein are administered at any interval desired.

Actual dosage levels of the human polyclonal antibodies or a pharmaceutical composition thereof may be varied so as to obtain an amount of the agent to achieve the desired therapeutic response for a particular subject, composition, and mode of administration, without being toxic to the subject. The selected dosage level can depend upon a variety of pharmacokinetic factors including the activity of the particular compositions of the present invention employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active agent calculated to produce the desired therapeutic effect in association with the required pharmaceutical carrier. The specification for the dosage unit forms of the disclosure can be determined by and directly dependent on (a) the unique characteristics of the active agent and the particular therapeutic effect to be achieved, and (b) the limitations inherent in the art of compounding such an active agent for the treatment of sensitivity in individuals. A dose can be determined by reference to a plasma concentration or a local concentration of the human polyclonal antibodies or pharmaceutical composition thereof.

A pharmaceutical composition described herein can be in a unit dosage form suitable for a single administration of a precise dosage. In unit dosage form, the formulation can be divided into unit doses containing appropriate quantities of the polyclonal antibodies. The unit dosage can be in the form of a package containing discrete quantities of the formulation. Non-limiting examples are packaged injectables, vials, and ampoules. An aqueous suspension composition disclosed herein can be packaged in a single-dose non-reclosable container. Multiple-dose reclosable containers can be used, for example, in combination with or without a preservative. A formulation for injection disclosed herein can be present in a unit dosage form, for example, in ampoules, or in multi dose containers with a preservative.

A dose is based on the amount of the agent per kilogram of body weight of a subject. A dose of an agent (e.g., antibody) is in the range of 10-3000 mg/kg, e.g., 100-2000 mg/kg, e.g., 300-500 mg/kg/day for 1-10 or 1-5 days; e.g., 400 mg/kg/day for 3-6 days; e.g., 1 g/kg/d for 2-3 days.

### Subjects

The human polyclonal antibodies or a pharmaceutical composition thereof is provided for use in treatment or prophylaxis of a condition disclosed herein, such as an infection with a microorganism. The human polyclonal antibodies or a pharmaceutical composition thereof can be administered to a subject that has the disease or condition. The human polyclonal antibodies or a pharmaceutical composition thereof can be administered as a prophylactic to subjects with a propensity to a condition or disease in order to lessen a likelihood of the occurrence of the disease or condition, or to reduce the severity of the disease or condition. In some embodiments, the human polyclonal antibodies or a pharmaceutical composition thereof induce an immune response against the antigen that the human polyclonal antibodies bind in a subject, thereby providing protection from the disease or infection in the subject. In some embodiments, the human polyclonal antibodies or a pharmaceutical composition thereof bind to a toxin in a subject (e.g., act as neutralizing human polyclonal antibodies), thereby providing protection from the toxin in the subject.

A subject can be a subject that is infected with a microorganism. A subject can be a subject that tested positive for the microorganism. A subject can be a subject that has been exposed to a microorganism. A subject can be a subject that has potentially been exposed to a microorganism. A subject can be a subject that is exhibiting one or more signs and/or symptoms consistent with infection with a microorganism.

A subject can be a subject having a cancer or tumor. A subject can be a subject that is at risk for a cancer or a tumor.

A subject can be a subject that has been exposed to a toxin. Exposure to a toxin can be from a bite or sting of a venomous animal, absorption of a toxin, inhalation of a toxin, ingestion of a toxin, or a drug overdose.

In some embodiments, a subject is a subject that is at high risk of coming into contact with a microorganism of the disclosure. For example, a subject may be a health care worker, a laboratory worker, or a first responder that is more likely to come into contact with a microorganism (e.g., a virus) of the disclosure. A subject may work at a health care facility, e.g., a hospital, doctor's surgery, inpatient facility, outpatient facility, urgent care facility, retirement home, aged care facility, or nursing home.

In some embodiments, a subject is a subject that is at high risk of complications if infected with a microorganism of the disclosure. For example, a subject can have a comorbidity, an age over 50, type 1 diabetes mellitus, type 1 diabetes mellitus, insulin resistance, or a combination thereof. In some embodiments, a subject is an immunocompromised subject. In some embodiments, a subject is on immunosuppressive drugs. In some embodiments, a subject is a transplant recipient that is on immunosuppressive drugs. In some embodiments, a subject is undergoing therapy for cancer, e.g., chemotherapy, that may decrease the function of the immune system.

A subject can be a human.

### EXAMPLES

The following examples are included for illustrative purposes only and are not intended to limit the scope of the invention.

### Example 1

### Design of Circular RNA Encoding Antigens

This example describes expression of antigens from circular RNA in non-human mammals with humanized immune systems. In this example, circular RNAs are designed to include an IRES, an ORF encoding an antigen, and two spacer elements flanking the IRES-ORF. Circularization enables rolling circle translation, multiple ORFs with alternating stagger elements for discrete ORF expression and controlled protein stoichiometry, and an IRES that targets RNA for ribosomal entry. Examples of antigens are:

A. Circular RNA Encoding SARS-CoV-2 Antigen

B. Circular RNA Encoding HER2 Antigen

The HER2 gene, which encodes the growth factor receptor HER2, is amplified and HER2 is overexpressed in 25 to 30 percent of breast cancers, increasing the aggressiveness of the tumor (Slamon D J et al., N Engl. J. Med., 344(11):783-792 (2001)). Monoclonal antibodies have antitumor activity against HER2-positive human breast tumor cells in laboratory models and can be used for the treatment of adult subjects with HER2-overexpressing breast cancers (Molina M A et al., Cancer Research, 61:4744-4749 (2001)). Examples of HER2 antigens are described in Ren X-R et al. (Breast Cancer Res., 14:R89 (2012)); and Morse M A et al. (Int. J. Cancer, 126:2893-2903 (2010)).

C. Circular RNA Encoding Methicillin-Resistant Staphylococcus aureus (MRSA) Antigen

MRSA infection is a multidrug resistant bacterium responsible for serious nosocomial and community-acquired infections. Numerous targets of S. aureus have been used in testing for immunotherapy. Specifically, PBP2a, a multi-modular class B penicillin-binding protein (PBP), located external to the membrane of all MRSA strains. Monoclonal antibody therapies have been shown in in vivo assays to be an effective prophylactic (Saraiva F B et al, PLoS ONE, 14(11): e0225752 (2019)).

D. Circular RNA Encoding Rabies Virus Antigen

Once clinical symptoms develop rabies is invariably fatal. Treatment is a post-exposure prophylaxis approach requiring immediate administration of rabies immunoglobulin with a course of rabies vaccination. Rabies viral epitopes have been used to develop monoclonal antibodies that neutralize the rabies virus (Bakker A B H et al. Vaccine, 26(47), 5922-5927 (2008)). Monoclonal antibody therapies have been shown in in vivo assays to be effective in neutralizing the virus (Bakker A B H et al., J. Virol., 79(14): 9062-9068 (2005); Marissen W E et al., J Virol., 79:4672-4678 (2005)).

E. Circular RNA Encoding Tumor Neoantigen

Neoantigens, which are derived from tumor-specific protein-coding mutations and are exempt from central tolerance, can generate robust immune responses and function as antigens that facilitate tumor rejection. Neoantigens from specific tumors (e.g., melanoma, glioblastoma), can be identified from tissues following surgical resection and peptide/nucleic acid analyses (Keskin D B et al., Nature, 565(7738):234-239 (2018)).

### Example 2

### Circular RNA Generation and Purification

In this example, circular RNAs were generated as follows. Unmodified linear RNA was synthesized by in vitro transcription using T7 RNA polymerase from a DNA segment. Transcribed RNA was purified with an RNA purification system (New England Biolabs), treated with RNA 5′phosphohydrolase (RppH) (New England Biolabs, M0356) following the manufacturer's instructions, and purified again with the RNA purification system. RppH-treated linear RNA was circularized using a splint DNA.

Splint-ligation was performed as follows: circular RNA was generated by treatment of the transcribed linear RNA and a DNA splint (5′-GTTTTTCGGCTATTCCCAATAGCCGTTTTG-3′) (SEQ ID NO: 23) using T4 DNA ligase 2 (New England Bio, Inc., M0239). To purify the circular RNAs, ligation mixtures were resolved on 4% denaturing PAGE and RNA bands corresponding to each circular RNA were excised. Excised RNA gel fragments were crushed, and RNA eluted with gel elution buffer (0.5 M Sodium Acetate, 0.1% SDS, 1 mM EDTA) for one hour at 37° C. Supernatant was harvested, and RNA was eluted again by adding gel elution buffer to the crushed gel and incubated for one hour. Gel debris was removed by centrifuge filters and was precipitated with ethanol. Agarose gel electrophoresis was used as a quality control measurement for validating purity and circularization.

### Example 3

### Expression of a Non-Secreted Protein or Antigen from Circular RNA in Mammalian Cells

To measure expression efficiency of non-secreted proteins or antigens from the RNA constructs, circular RNA (0.1 picomole) encoding a protein or antigen is designed and produced by the methods described herein. Circular RNA is transfected into HEK293 (10,000 cells per well in a 96 well plate in serum-free media) using MessengerMax (Invitrogen, LMRNA).

For a non-secreted protein or antigen (e.g. SARS-CoV-2 spike antigen, HER2 antigen, neoantigen), protein expression is measured using an antigen-specific ELISA (e.g., SARS-CoV-2 spike antigen-specific ELISA, HER2-specific ELISA) at 24, 48, and 72 hours. To measure expression, cells are lysed in each well at the appropriate timepoint, using a lysis buffer and a protease inhibitor. The cell lysate is retrieved and centrifuged at 12,000 rpm for 10 minutes. Supernatant is collected.

For SARS-Co-2 spike antigen, a SARS-CoV-2 2019 spike antigen detection sandwich ELISA kit is used (SARS-CoV-2 (2019-nCoV) Spike Detection ELISA Kit, Sino Biological, KIT40591) according to the manufacturer's instructions.

### Example 4

### Expression of a Secreted Protein or Antigen from Circular RNA in Mammalian Cells

To measure expression efficiency of secreted proteins or antigens from the RNA constructs, circular RNA (0.1 picomole) encoding a protein or antigen is designed and produced by the methods described herein. Circular RNA is transfected into HEK293 (10,000 cells per well in a 96 well plate in serum-free media) using MessengerMax (Invitrogen, LMRNA).

For a secreted protein or antigen (e.g., secreted rabies antigen, PBP2a antigen, neoantigen), antigen expression is detected using an antigen-specific Western blot at 24, 48, and 72 hours. Briefly, 80 uL of supernatant from mammalian cells is taken from each well. Protein levels in harvested media is measured by BCA protein assay method and same amount of protein is resolved on 4%-12% gradient Bis-Tris gel (Thermo Fisher Scientific) and transferred to nitrocellulose membrane using by iBlot2 transfer system (Thermo Fisher Scientific). Anti-antigen antibody (Sino Biological) is used to detect the antigen. The chemiluminescence signal from protein bands is monitored by iBright FL1500 imaging system (Invitrogen).

### Example 5

### Expression of RBD Antigen from Circular RNA in Mammalian Cells

This example demonstrates the ability to express RBD antigens from circular RNA in mammalian cells.

In this Example, circular RNAs encoding SARS-CoV-2 RBD antigens were designed, produced, and purified by the methods described herein.

The expression of RBD-encoding circular RNA was tested by immunoprecipitation coupled with Western blot (IP-Western). Briefly, circular RNA encoding an RBD antigen (0.1 picomoles) was transfected into BJ Fibroblasts and HeLa cells (10,000 cells) using Lipofectamine MessengerMax (ThermoFisher, LMRNA015). MessengerMax alone was used as a control. Supernatant was collected at 24 hours and immunoprecipitation was performed with a rabbit antibody specific to the SARS-CoV-2 RBD-Spike Glycoprotein (Sino Biologicals, Cat: 40592-T62) coupled to Protein G-Dynabeads (Invitrogen, 10003D) and the same antibody was used to detect the immunoprecipitated products resolved by PAGE. A recombinant RBD (42 ng) Immunoprecipitation was used as control and to quantify cell protein expression. Membrane chemiluminescence was quantified using the Image Studio™ Lite western blot quantification software (Li-COR Biosciences).

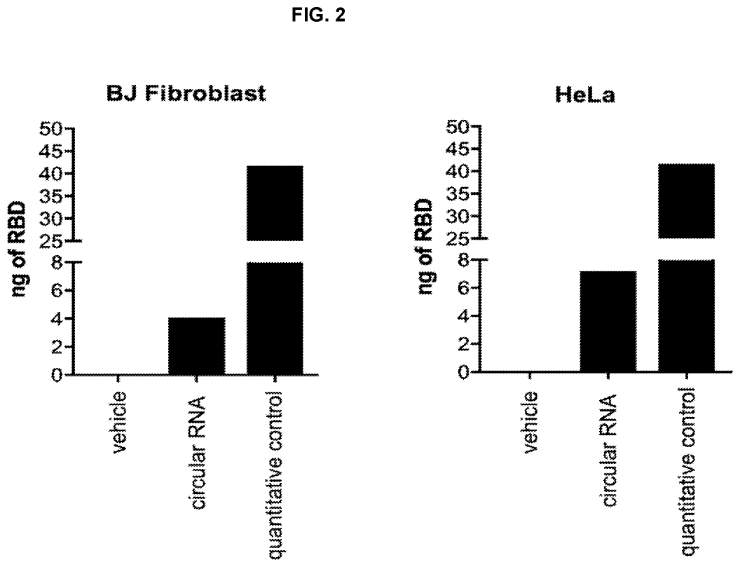

RBD antigen encoded by circular RNA was detected in BJ Fibroblast and HeLa cell supernatants and not in the controls (FIG. 2).

This Example shows that SAR-CoV-2 RBD antigens (which are secreted proteins) were expressed from circular RNA in mammalian cells.

### Example 6

### Formulation of Circular RNA for Administration to Non-Human Animal

Circular RNAs encoding SARS-CoV-2 antigens were designed by the methods described herein. Circular RNAs encoding Gaussia Luciferase (GLuc) was designed with an IRES and ORF encoding a GLuc polypeptide (SEQ ID NO: 24). Circular RNAs were produced and purified by the methods described herein. After purification, the circular RNA was formulated as follows:

A. circular RNA was diluted in PBS to a final concentration of 2.5 or 25 picomoles in 50 uL, to obtain a circular RNA preparation (unformulated).

B. circular RNA was formulated with a lipid carrier (e.g., TransIT (Minis Bio)) and mRNA Boost Reagent (Minis Bio) according to the manufacturer's instructions (15% TransIT, 5% Boost) to obtain a final RNA concentration of 2.5 or 25 picomoles in 50 uL, to obtain a circular RNA preparation.

C. circular RNA was formulated with a cationic polymer (e.g., protamine). Briefly, circular RNA was diluted in pure water to a concentration of 1100 ng/uL. Protamine sulfate was dissolved in Ringer lactate solution (4000 ng/uL). While stirring, the protamine-Ringer lactate solution was added to half of the circular RNA solution until a weight ratio of RNA:protamine is 2:1. The solution was stirred for another 10 minutes to ensure the formation of stable complexes. The remaining circular RNA was then added, and the solution stirred briefly. The final concentration of the solution was adjusted using Ringer lactate solution such that the final concentration of circular RNA was 2.5 or 25 picomoles in per 50 uL.

### Example 7

### Administration and Collection of Blood of Circular RNA to Non-Human Animal

In this example, mice received 50 uL injections of each circular RNA preparation via either a single intramuscular injection in a hind leg or a single intradermal injection to the back. Blood samples (˜25 μL) were collected from each mouse for analysis by submalar drawing. Blood was collected into EDTA tubes, at 0, 6 hours, 24, 48 hours and 7 days post-dosing of the circular RNA. Plasma was isolated by centrifugation for 30 minutes at 1300 g at 4° C.

### Example 8

### Detection of Antibodies to Antigen

This example describes how to determine the presence of antibodies to antigen.

A. To detect an antibody to the SARS-CoV-2 RBD antigen, an ELISA is used as described by Chen X et al. (medRxiv, doi: doi.org/10.1101/2020.04.06.20055475 (2020)). Briefly, SARS-CoV-2 spike RBD protein (Sino Biological, 40592-V08B) in 100 uL PBS per well is coated on ELISA plates overnight at 4° C. ELISA plates are then blocked for 1 hour with blocking buffer (5% FBS plus 0.05% Tween 20). 10-fold diluted plasma is then added to each well in 100 uL blocking buffer over 1 hour. After washing with 1× phosphate-buffered saline with Tween® detergent (PBST), bound antibodies are incubated with anti-mouse IgG HRP detection antibody (Invitrogen) for 30 mins, followed by wash with PBST, then PBS, and addition of tetramethylbenzene. The ELISA plate is allowed to react for 5 min and then quenched using 1 M HCl Stop buffer. The optical density (OD) value is determined at 450 nm.

B. To detect an antibody to the SARS-CoV-2 spike antigen, an ELISA is used as described by Chen X et al. (medRxiv, doi: doi.org/10.1101/2020.04.06.20055475 (2020)). Briefly, SARS-CoV-2 spike antigen protein (Sino Biological, 40591-V08H) in 100 uL PBS per well is coated on ELISA plates overnight at 4° C. ELISA plates are then blocked for 1 hour with blocking buffer (5% FBS plus 0.05% Tween 20). 10-fold diluted plasma is then added to each well in 100 uL blocking buffer over 1 hour. After washing with 1× phosphate-buffered saline with Tween® detergent (PBST), bound antibodies are incubated with anti-mouse IgG HRP detection antibody (Invitrogen) for 30 mins, followed by wash with PBST, then PBS, and addition of tetramethylbenzene. The ELISA plates are allowed to react for 5 min and then quenched using 1 M HCl Stop buffer. The optical density (OD) value is determined at 450 nm.

### Example 9

### Evaluation of Neutralizing Activity of Antibodies

A SARS-CoV-2 viral neutralization assay is used to test neutralization ability of antibodies against SARS-CoV-2 infection. An example of such an assay is described by Okba N M A et al. (Emerg Infect Dis., doi: 10.3201/eid2607.200841 (2020)). This assay detects the production of antibodies that functionally inhibit viral infection demonstrated by a reduction in the number of viral plaques. Slight variations of this assay are described in Gauger P C & Vincent A L (in Animal Influenza Virus: Methods and Protocols, 3rd edition, ed. E. Spackman, pp. 311-320 (2014)) and Wilson H L et al. (J. Clin. Microbiol., 55(10):3104-3112 (2017)). Briefly, in this example, a SARS-CoV-2 viral neutralization assay is used to determine the neutralization ability of plasma containing anti-SARS-CoV-2 antibodies produced by non-human animals in response to inoculation with circular RNA encoding SARS-CoV-2 antigens. Plasma from naïve animals injected with vehicle only (no circular RNA) is used as a control.

### Example 10

### Immunogenicity of SARS-CoV-2 RBD Antigens in Mouse Model

The immunogenicity of a circular RNA encoding a SARS-CoV-2 RBD antigen, formulated with a cationic polymer (e.g., protamine), was evaluated in a mouse model. Production of antibodies to a SARS-CoV-2 RBD antigen, formulated with the cationic polymer, was also evaluated in the mouse model.

In this example, circular RNA was designed with an IRES and ORF encoding a SARS-CoV-2 RBD antigen by the methods described herein. Unmodified linear RNA was synthesized by in vitro transcription with an excess of guanosine 5′ monophosphate using T7 RNA polymerase from a DNA segment. Transcribed RNA was purified with an RNA purification system (New England Biolabs, Inc.) following the manufacturer's instructions. Purified linear RNA was circularized using a splint DNA.

Circular RNA was generated by split-ligation as follows: Transcribed linear RNA and a DNA splint (5′-GTTTTTCGGCTATTCCCAATAGCCGTTTTG-3′) (SEQ ID NO: 23) were mixed and annealed and treated with an RNA ligase. To purify the circular RNAs, ligation mixtures were resolved by reverse-phase chromatography. Circular RNA was selectively eluted from linear RNA by increasing the organic content of the mobile phase. Eluted RNA was fractionally collected and assayed for circular RNA purity. Selected fractions were combined and buffer exchanged to remove mobile phase salts and solvents. Acrylamide gel electrophoresis was used as a quality control measurement for validating purity and circularization.

The purified circular RNA was diluted in pure water to a concentration of 1100 ng/uL. Protamine sulfate was dissolved in Ringer's lactate solution (4000 ng/uL). While stirring, the protamine-Ringer lactate solution was added to half of the circular RNA solution until a weight ratio of RNA:protamine is 2:1. The solution was stirred for another 10 minutes to ensure the formation of stable complexes. The remaining circular RNA was then added (i.e., remaining circular RNA to circular RNA:protamine solution) and the solution stirred briefly. The final concentration of the mixture (i.e., circular RNA mixture) was adjusted using Ringer's lactate solution to obtain a circular RNA preparation with a final RNA concentration of 2 ug or 10 ug of RNA in 50 uL.

Three mice per group were vaccinated intramuscularly or intradermally with a 2 ug or 10 ug dose of the circular RNA preparation, or a protamine vehicle control at day 0 and day 21. Addavax™ adjuvant (Invivogen) was administered once to each mouse, intramuscularly or intradermally, 24 hours after administration of the circular RNA preparation at day 0 and day 21. Addavax™ adjuvant was dosed at 50% in 1×PBS in 50 uL following to the manufacturer's instructions.

Blood collection from each mouse was by submalar drawing. Blood was collected into dry-anticoagulant free-tubes, at day 7, 14, 21, 23, 28, 35, 41, 49, 56, 63, 69, 77, 84, 108 and 115 days post-dosing of the circular RNA. Serum was separated from whole blood by centrifugation at 1200g for 30 minutes at 4 C. The serum was heat-inactivated by heating at 56° C. for 1 hour. Individual heat-inactivated serum samples were assayed for the presence of RBD-specific IgG by enzyme-linked immunosorbent assay (ELISA). ELISA plates (MaxiSorp 442404 96-well, Nunc) were coated overnight at 4° C. with SARS-CoV-2 RBD (Sino Biological, 40592-V08B; 100 ng) in 100 uL PBS. The plates were then blocked for 1 hour with blocking buffer (TBS with 2% FBS and 0.05% Tween 20). Serum dilutions were then added to each well in 100 uL blocking buffer and incubated at room temperature for 1 hour. After washing three times with 1× Tris-buffered saline with Tween® detergent (TBS-T), plates were incubated with anti-mouse IgG HRP detection antibody (Jackson 115-035-071) for 1 hour followed by three washes with TBS-T, then addition of tetramethylbenzene (Pierce 34021). The ELISA plate was allowed to react for 5 min and then quenched using 2N sulfuric acid. The optical density (OD) value was determined at 450 nm.

The optical density of each serum sample was divided by that of the background (plates coated with RBD, incubated only with secondary antibody). The fold over background of each sample was plotted.

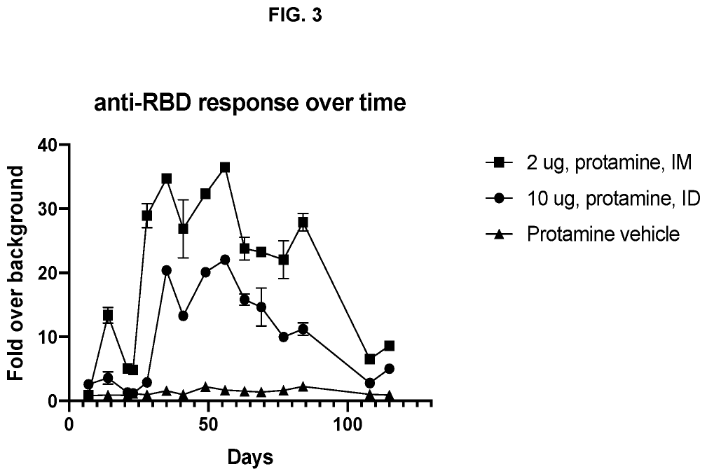

The results showed that anti-RBD antibodies were obtained at days 14, 21, 23, 28, 35, 41, 49, 56, 63, 69, 77, 84, 108 and 115 after injection with the circular RNA preparations (FIG. 3). Anti-RBD antibodies were not obtained after injection with the protamine vehicle. These results also showed that circular RNA encoding the RBD antigen induced an antigen-specific immune response in mice.

A similar ELISA was used to assay serum samples for the presence of Spike-specific IgG. ELISA plates (MaxiSorp 442404 96-well, Nunc) were coated overnight at 4° C. with SARS-CoV-2 Spike (Sino Biological, 40589-V08B1; 100 ng) in 100 uL PBS. The plates were then blocked for 1 hour with blocking buffer (TBS with 2% FBS and 0.05% Tween 20). Serum dilutions were then added to each well in 100 uL blocking buffer and incubated at room temperature for 1 hour. After washing three times with 1× Tris-buffered saline with Tween® detergent (TBS-T), plates were incubated with anti-mouse IgG HRP detection antibody (Jackson 115-035-071) for 1 hour followed by three washes with TBS-T, then addition of tetramethylbenzene (Pierce 34021). The ELISA plate was allowed to react for 5 min and then quenched using 2N sulfuric acid. The optical density (OD) value was determined at 450 nm.

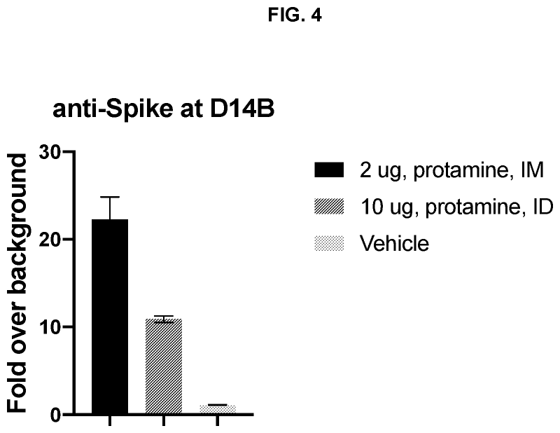

The results showed that anti-Spike antibodies were obtained at 35 days after injection with the circular RNA preparations (FIG. 4). Anti-Spike antibodies were not obtained after injection with vehicle.

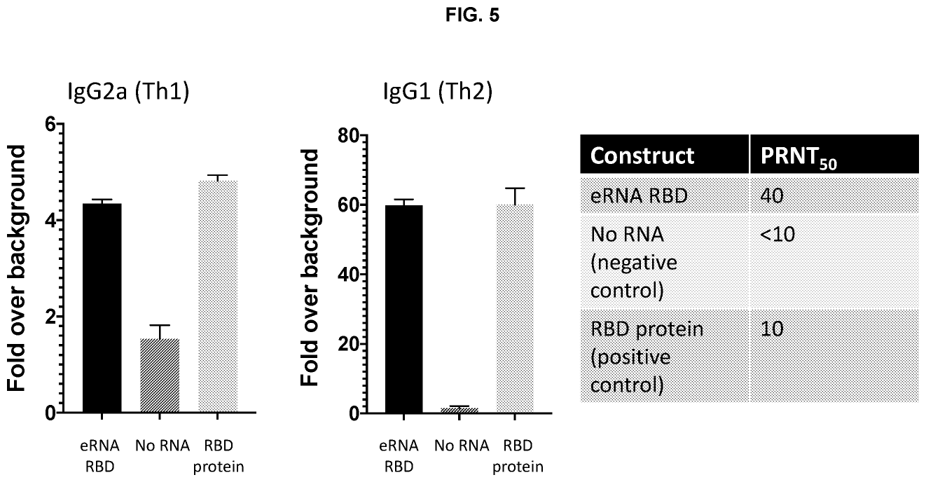

Serum antibodies at day 14 post-dosing were characterized using an assay to measure relative IgG1 vs IgG2a isotypes (FIG. 5), and the ability of serum antibodies to neutralize the virus was characterized using a PRNT neutralization assay. The results showed that 2 ug RBD eRNA dosed intramuscularly with adjuvant had neutralizing ability.

### Example 11

### Modulation of In Vivo Production of Gaussia Luciferase from Circular RNA in Mice Using Timed of Adjuvant Delivery

This example demonstrates the expression of proteins from circular RNA in vivo whilst also delivering an adjuvant to stimulate an immune response.

In this example, circular RNA encoding GLuc was designed as described in Example 6 and produced and purified by the methods described herein. Circular RNAs were formulated as described in Example 6 to obtain circular RNA preparations (e.g., Trans-IT formulated, protamine formulated, PBS/unformulated). Mice were administered each circular RNA preparation intramuscularly as described in Example 7. Another group of mice were administered a protamine formulated circular RNA preparation intradermally as described in Example 7.

To stimulate the immune response, Addavax™ adjuvant (Invivogen), which is a squalene-based oil-in-water nano-emulsion with a formulation similar to MF59® adjuvant, was injected into the mouse hind leg at 0 hours (simultaneous delivery with a circular RNA preparation) or at 24 hours. Addavax™ adjuvant was dosed at 50 uL according to the manufacturer's instructions.

Blood samples (˜25 μL) were collected from each mouse by submalar drawing. Blood was collected into EDTA tubes, at 0, 6, 24 and 48 hours post-dosing of the circular RNA. Plasma was isolated by centrifugation for 30 minutes at 1300 g at 4° C. and the activity of Gaussia Luciferase, a secreted enzyme, was tested using a Gaussia Luciferase activity assay (Thermo Scientific Pierce). 50 μL of 1× GLuc substrate was added to 5 μL of plasma to carry out the GLuc luciferase activity assay. Plates were read immediately after mixing in a luminometer instrument (Promega).

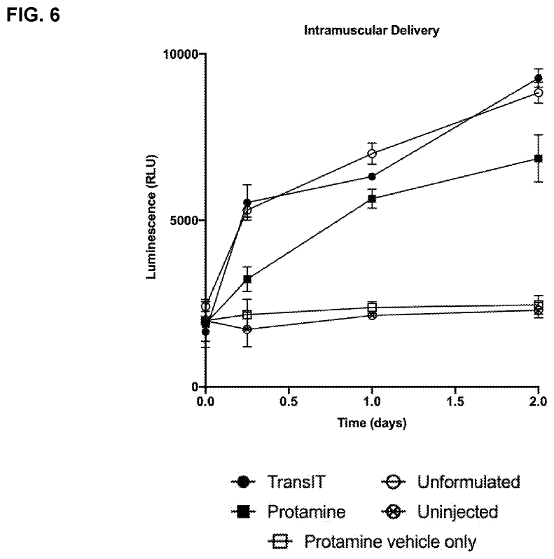

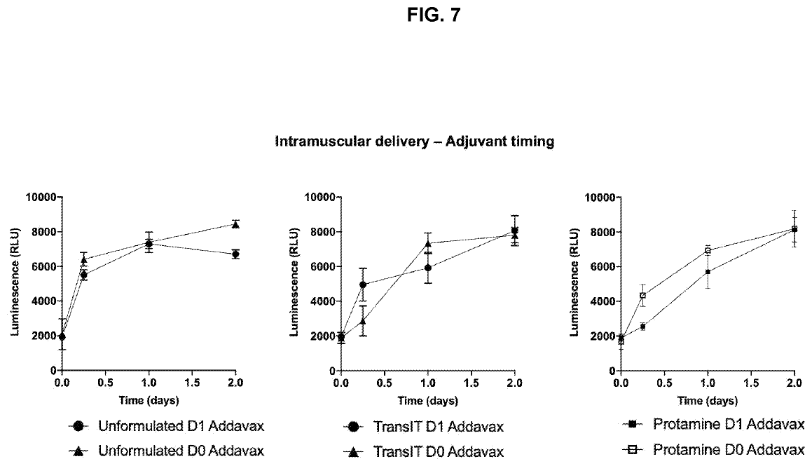

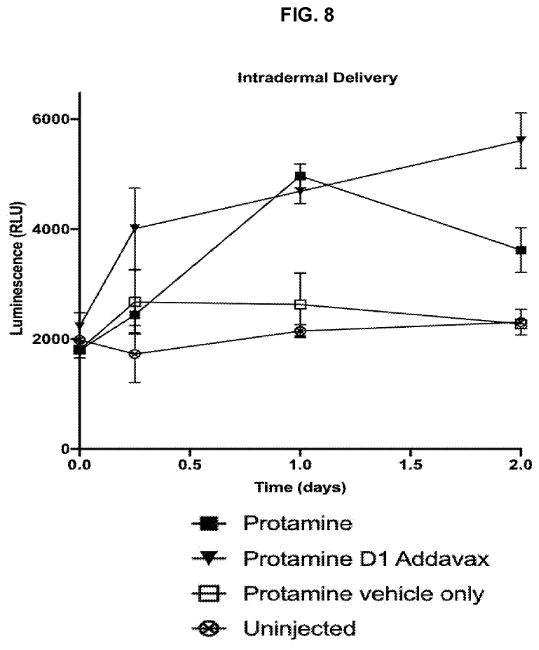

This example demonstrated successful protein expression from circular RNA in vivo for prolonged periods of time using: (a) intramuscular injection of TransIT formulated, protamine formulated and unformulated circular RNA preparations without adjuvant (FIG. 6), and with adjuvant delivered at 0 and 24 h (FIG. 7); and (b) intradermal injection of protamine formulated circular RNA preparation without adjuvant, and with adjuvant delivered at 24 h (FIG. 8).

### Example 12

### Administration of Circular RNA Encoding an Antigen Formulated with Lipid Carrier to a Tc Bovine

For this example, circular RNAs encoding antigens are designed by the methods described herein, produced, and purified by the methods described herein.

In this example, circular RNA is formulated with a lipid carrier (e.g., TransIT (Minis Bio)) and mRNA Boost Reagent (Minis Bio), as described in Example 6 for circular RNAs encoding SARs-CoV-2 antigens. Total volume of 8 mL is generated, corresponding to 2 nanomoles of circular RNA. Circular RNA is formulated to obtain a circular RNA preparation shortly before injection into animals.

In this example, transchromosomal (Tc) bovines in which bovine immunoglobulin genes are knocked out and a human artificial chromosome containing the full germ-line sequence of human immunoglobin was inserted, are used. This allows the Tc bovines to produce target-specific full human antibodies upon injection (see Matsushita et al. PLoS One. 10:6 (2015) and Fuentes S et al. (J Infect. Dis., 218(Suppl 5): S597-S602 (2018)). Tc bovines have been engineered to possess a human artificial chromosome containing the human antibody heavy chain and kappa chain. These animals have a triple deletion of bovine heavy chain genes and lambda cluster light chain genes (IGHM^{-/-} IGHML1^{-/-} IGL^{-/-}). Tc bovines produce three kinds of IgG antibody: human IgG (hIgG), chimeric IgG (containing human heavy chain and bovine kappa chain), and trans-class-switched bovine IgG. The majority of antibody produced is fully human IgG.

In this example, Tc bovines are immunized with a circular RNA preparation or vehicle only control (i.e. a no circular RNA control) via intramuscular injection or intradermal injection.

A. Intramuscular injection: A total of 4 injections are administered at the following sites: one (1) injection of 2 mL (each) behind each ear; and one (1) injection of 2 mL (each) to each hind leg.

B. Intradermal injection: A total of 4 injections are administered at the following sites: 4 injections of 2 mL to individual sites at the neck-shoulder border.

Prior to the first injection (V1), a volume of pre-injection plasma is collected from each study Tc bovine to be used as a negative control.

### Example 13

### Expression of Antigens from Circular RNA in Tc Bovine

A. For secreted proteins (e.g., SARS-CoV-2 RBD antigen, secreted rabies antigen, PBP2a antigen, neoantigen), blood samples, up to 2.1% of the bovine's body weight, are collected via jugular venipuncture at days 1, 3, 5, 7, 14 and 21 post-injection. Plasma is collected using an automated plasmapheresis system (Baxter Healthcare, Autopheresis C Model 200). Plasma is then verified for expression of antigens as described in Example 4, e.g. expression of RBD antigen is assessed by ELISA performed as described in Example 4. For these ELISAs, an anti-human IgG HRP detection antibody (Invitrogen) is used.

B. For a non-secreted protein (e.g., SARS-CoV-2 spike antigen, HER2 antigen, neoantigen), tissues are harvested for analysis of protein expression at one or more time points post-dosing, such as for example at one or more of 2, 5, 7 and 21 days post-dosing. Tc bovine is sacrificed and muscle (from the site of injection) is harvested and analyzed for antigen expression as described in Example 3. Expression of antigen is assessed by protein-specific ELISA (e.g. HER2-specific ELISA) performed on protein extracted from each tissue.

### Example 14

### Production of Human Polyclonal Antibodies Against Disease-Relevant Antigens from Circular RNA Encoding Antigens in Tc Bovine

This example describes production of fully human neutralizing polyclonal antibodies to a disease-relevant antigen in non-human mammals with humanized immune system from circular RNA encoding the disease-relevant antigen.

For this example, circular RNAs encoding an antigen are designed, produced, and purified by the methods described herein.

In this example, in one approach, circular RNA is formulated as described in Example 6, for circular RNAs encoding SARS-CoV-2 antigens (e.g., formulated with a lipid carrier, formulated with a cationic polymer or unformulated), to obtain a first set of circular RNA preparations. In a second approach, Addavax™ adjuvant, MF59 adjuvant, complete Freund's adjuvant or SAB's proprietary adjuvant formulation (SAB-adj-1) is formulated with the circular RNA-lipid carrier mixture or the unformulated circular RNA preparation, as described in Beigel J H et al. (Lancet Infect. Dis., 18: 410-418 (2018)), to obtain a second set of circular RNA preparations with a final concentration of circular RNA of 25 picomoles in 100 uL. For each approach, a total volume of 8 mL is generated, corresponding to 2 nanomoles of circular RNA. Circular RNA is formulated to obtain the circular RNA preparations shortly before injection into animals.

In this example, Tc bovine are immunized with the circular RNA preparations or a vehicle only control (i.e. no circular RNA control) via intramuscular or intradermal injection.

A. Intramuscular injection: A total of 4 injections are administered at each time point at the following sites: one injections of 2 mL (each) behind each ear; and one injection of 2 mL (each) to either side of the neck.

B. Intradermal injection: A total of 4 injections are administered at each time point at the following sites: four injections of 2 mL to individual sites at the neck-shoulder border.

A total of 8 timepoints are used: 0, 3, 6, 9, 12, 15, 18 and 21 weeks.

Where the first set of circular RNA preparations is administered, Addavax™ adjuvant, MF59® adjuvant, complete Freund's adjuvant or SAB's SAB-adj-1 is separately administered adjacent (1-2 cm) to each injection site (2 mL total) for the first 3 timepoints. Prior to the first injection (V1), a volume of pre-injection plasma is collected from each study Tc bovine to be used as negative control. Blood samples, up to 2.1% of the bovine's body weight, are collected via jugular venipuncture at days 8, 9, 10, 11, 12 and 14 days post-injection at each timepoint and at an additional timepoint, 60 days, post-final injection. Plasma is collected using an automated plasmapheresis system (Baxter Healthcare, Autopheresis C Model 200). Plasma is then verified for antigen-specific antibodies using an antigen-based ELISA. Human polyclonal antibodies are purified from the plasma using Cohn-Oncley purification and Caprylate fractionation, for antigen-specific polyclonal antibodies as described below in Example 16.

### Example 15

### Production of Human Polyclonal Antibodies Against Disease-Relevant Antigens from Circular RNA Encoding Antigens in Tc Caprine

For this example, circular RNAs encoding disease-relevant antigens were designed, produced, and purified by the methods described herein.

In this example, circular RNA as described in Example 6, for circular RNAs encoding SARS-CoV-2 antigens (e.g., formulated with a lipid carrier, formulated with a cationic polymer or unformulated), to obtain circular RNA preparations. The final RNA concentration is 25 picomoles in 100 uL. Total volume of 1 mL is generated, corresponding to 0.25 nanomoles of circular RNA. Circular RNA is formulated to obtain a circular RNA preparation shortly before injection into animals. For a total of 4 injections, a total of 4 mL of circular RNA is formulated.

In this example, a transchromosomal goats (Tc caprine), in which a human artificial chromosome (HAC) comprising the entire human immunoglobulin (Ig) gene repertoire in the germline configuration was introduced into the genetic makeup of the domestic goat, are used. Tc caprine produces human polyclonal antibodies in their sera (see Wu H et al. (Sci Rep, 9(1): 366, doi: doi.org/10.1038/s41598-018-36961-5 (2019)).

In this example, Tc caprine are immunized with a circular RNA preparation or a vehicle only control (i.e., a no circular RNA control) via intramuscular or intradermal injection.

A. Intramuscular injection. A total of 2 injections are administered at each time point at the following sites: one injection of 0.5 mL (each) to either side of the neck.

B. Intradermal injection. A total of 2 injections are administered at each time point at the following sites: one injection of 0.5 mL (each) to opposing sides of the lower neck-shoulder.

A total of 4 timepoints are used: 0, 3, 6 and 9 weeks.

Addavax™ adjuvant (Invivogen), MF59® adjuvant, complete Freund's adjuvant or SAB's proprietary adjuvant formulation (SAB-adj-1) is administered adjacent (1-2 cm) to each injection site (0.5 mL total) for the first 3 timepoints.

Blood samples (40 mL) are collected via jugular venipuncture at days 8 and 14 post-injection at each timepoint and at an additional timepoint, 60 days, post-final injection. Plasma is collected using an automated plasmapheresis system (Baxter Healthcare, Autopheresis C Model 200). Plasma is then verified for antigen-specific antibodies.

### Example 16

### Purification of Polyclonal Antibodies

This example describes purification of human polyclonal antibodies from plasma of non-human mammal with a humanized immune system.

For this example, neutralizing human polyclonal antibodies against a disease-relevant antigen are produced as described in Example 14 and Example 15.

For purification of human polyclonal antibodies (e.g., anti-SARS-CoV-2 polyclonal antibodies) from collected plasma and subsequent use in human subjects, protein antigen-inactivation and removal are required. In this example, human polyclonal antibodies (e.g., human polyclonal anti-SARS-CoV-2 antibodies) are purified from plasma using the Cohn-Oncley method as described in (Ofosu et al. F A (Thromb. Haemost., (2008)); Buchacher A & Iberer G (Biotechnol. J., 1(2): 148-163 (2006)); Buchacher A & Curling J M (in Biopharm. Process., Chap 42, pp. 857-876, doi: https://doi.org/10.1016/B978-0-08-100623-8.00043-8 (2018)). Fraction (I+) II+III obtained by the Cohn-Oncley method is collected, and human polyclonal antibodies (e.g., human polyclonal anti-SARS-CoV-2 antibodies) are purified from this fraction using methods described by Lebing W et al. (Vox Sanguinis, 84(3):193-201 (2003)). Briefly, Fraction II+III is suspended in 12 volumes of water for injection (WFI) at pH 4.2. Sodium caprylate (20 mM) is added and pH is adjusted to pH 5.1 with sodium hydroxide. During this step, lipoproteins, albumin and a portion of caprylate is precipitated. The precipitate is removed by cloth filtration in the presence of filter aid. After filtration, the caprylate concentration is readjusted to 20 mM and the solution is incubated at pH 5.1 for 1 hour at 25° C., to inactivate enveloped virus. The solution is clarified by depth filtration with filter aid. The filtrate is then passed through two successive anion-exchange chromatography columns (Q Sepharose FF followed by ANX Sepharose FF) at pH 5.2. The eluate is concentrated by ultrafiltration (BioMax 50 KDa cassettes, Millipore) and diafiltered against WFI using the same system. The purified IgG solution is adjusted to pH 4.25, 0.2 M glycine and 100 mg/mL protein. Bulk IVIG is sterile filtered and used to fill 10, 50, 100, or 200 mL vials. The final product is incubated for 21 days at 23-27° C. for virus inactivation before storage at 2-8° C.

To verify enrichment of the IVIG, cellulose acetate electrophoresis is used. For clinical use, 95% purity is typical.

### Example 17

### Formulation of Fully Human Polyclonal Antibodies for Treatment of Human Subjects

In this example, purified antibodies are formulated at neutral pH (pH 7.2) and diluted in an ionic solution containing sodium chloride. A United States Pharmacopoeia (USP) grade infusion solution, 0.9% sodium chloride, is used. The clinical formulation can be based on a few solution compositions which include:
1. Trehalose, sodium citrate, citric acid, polysorbate 80.
2. Sodium succinate, sucrose, polysorbate 20.
3. Sodium chloride, tromethamine, polysorbate 80.
4. Sucrose, sodium chloride, sodium phosphate, dextran 40.

### Example 18

### Treatment of Human Subjects with a HER2-Positive Cancer

This example describes administration of fully human anti-HER2 polyclonal antibodies to adult human subjects with a HER2-positive cancer (e.g., breast cancer, ovarian cancer, colon cancer, pancreatic cancer or gastric cancer).

In this example, treatment of adult human subjects with a HER2-positive cancer is exemplified with adult human subjects with breast cancer. Purified human polyclonal antibodies against HER2 are obtained as described in Example 14 and formulated as described in Example 17.

In this example, adult breast cancer subjects with HER2-overexpressing tumors are administered formulated polyclonal antibodies at a dose of 8 mg/kg. Weekly doses are administered over 12 weeks as described in Fountzilas G et al. (Annals of Oncology, 12(11):1545-1551 (2001)). The effect of the polyclonal antibodies on treated subjects is assessed by evaluating markers of disease progression as described in Tokuda Y et al. (British J. Cancer, 81(8):1419-1425 (1999)); and Fountzilas G et al. (Annals of Oncology, 12(11):1545-1551 (2001)).

Treated subjects are monitored using one or more markers of disease progression.

### Example 19

### Treatment of Human Subjects Susceptible to Infection by Methicillin-Resistant Staphylococcus Aureus with Human Polyclonal Antibodies Produced in Tc Bovine from Circular RNA Formulated with Lipid Carrier

This example describes the administration of fully human polyclonal antibodies for treatment of human subjects susceptible to infection by methicillin-resistant Staphylococcus aureus (MRSA).

In this example, human polyclonal antibodies against MRSA are produced as described in Example 14, and purified antibodies are formulated as described in Example 17.

In this example, MRSA-susceptible human subjects (e.g. medically compromised human subjects in a hospital, nursing home or dialysis center) or health care workers are administered formulated anti-PBP2a polyclonal antibodies at a dose of 40 mg per week. The effect of the polyclonal antibodies on treated subjects is assessed by evaluating rates of infection with MRSA by tracking various known markers of disease progression, including, for example, as described in Gordon R J & Lowy F D (Clin Infect Dis., 46(Suppl 5):S350-S359 (2008)).

### Example 20

### Treatment of Human Subjects Against Rabies Virus

This example describes administration of fully human polyclonal antibodies against RVG to human subjects susceptible to infection by rabies virus.

In this example, human polyclonal antibodies against RVG are produced as described in Example 13, and purified antibodies are formulated as described in Example 17.

In this example, human subjects with high risk exposure to rabies or rabid animals are administered formulated human polyclonal antibodies at a dose of 40 mg per week. The effect of the polyclonal antibodies on treated human subjects is assessed by tracking rabies virus neutralizing ability (Bakker A B H et al., Vaccine, 26(47):5922-5927 (2008)) and rates of infection with rabies.

### Example 21

### Treatment of Human Subjects with Cancer

This example describes administration of fully human polyclonal antibodies to adult human subjects with cancer.

In this example, treatment of adult human cancer subjects is exemplified using human subjects with advanced melanoma. The method described in this example can be readily adapted and applied to treatment of human subjects with other cancer tumors.

In this example, neoantigen-specific human polyclonal antibodies are produced as described in Example 14, and purified antibodies are formulated as described in Example 17.

In this example, human subjects with melanoma are administered formulated neoantigen-specific polyclonal antibodies at a dose of 3 mg/kg, 3 times per week (as described in Hendrikx J J M A et al., Oncologist, 22(10):1212-1221 (2017)). The effect of the polyclonal antibodies on treated subjects is assessed by evaluating various disease metrics described in the art, including, for example in Ott P A et al. (Nature, 547, 217-221 (2017)).

### Example 22

### Treatment of a Human Subject After a Snake Bite

This example describes administration of fully human polyclonal antibodies to an adult human subject after being bitten by a venomous snake.

In this example, treatment is exemplified using an adult human subject after being bitten by a king cobra. The method described in this example can be readily adapted and applied to treatment of human subjects after being bitten or stung by other types of venomous animals.

In this example, cytotoxin-specific human polyclonal antibodies are produced as described in Example 14, and purified antibodies are formulated as described in Example 17.

In this example, the human subject that was bitten by the venomous snake is administered formulated cytotoxin-specific polyclonal antibodies.

### Example 23

### Treatment of a Human Subject After Ingestion of a Toxin

This example describes administration of fully human polyclonal antibodies to adult human subject after ingestion of a toxin.

In this example, treatment is exemplified using an adult human subject after ingestion of a mushroom comprising a mycotoxin. The method described in this example can be readily adapted and applied to treatment of human subjects after ingestion of a toxin.

In this example, mycotoxin-specific human polyclonal antibodies are produced as described in Example 14, and purified antibodies are formulated as described in Example 17.

In this example, the human subject that ingested the mushroom comprising the mycotoxin is administered formulated mycotoxin-specific polyclonal antibodies.

### Example 24

### Treatment of a Human Subject After Toxicity from a Drug Overdose

This example describes administration of fully human polyclonal antibodies to adult human subject having toxicity associated with a drug overdose.

In this example, treatment is exemplified using a human subject after a drug overdose of digoxin. The method described in this example can be readily adapted and applied to treatment of human subjects after toxicity associated with a drug overdose.

In this example, digoxin-specific human polyclonal antibodies are produced as described in Example 14, and purified antibodies are formulated as described in Example 17.

In this example, the human subject that overdosed on digoxin is administered formulated digoxin-specific polyclonal antibodies.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Sequences References in the Examples

WT EMCV GLuc IS
SEQ ID NO: 24
CGCGGATCCTAATACGACTCACTATAGGGAATAGCCGAAAAACAAAAAAC
AAAAAAAACAAAAAAAAAACCAAAAAAACAAAACACAACGTTACTGGCCG
AAGCCGCTTGGAATAAGGCCGGTGTGCGTTTGTCTATATGTTATTTTCCA
CCATATTGCCGTCTTTTGGCAATGTGAGGGCCCGGAAACCTGGCCCTGTC
TTCTTGACGAGCATTCCTAGGGGTCTTTCCCCTCTCGCCAAAGGAATGCA
AGGTCTGTTGAATGTCGTGAAGGAAGCAGTTCCTCTGGAAGCTTCTTGAA
GACAAACAACGTCTGTAGCGACCCTTTGCAGGCAGCGGAACCCCCCACCT
GGCGACAGGTGCCTCTGCGGCCAAAAGCCACGTGTATAAGATACACCTGC
AAAGGCGGCACAACCCCAGTGCCACGTTGTGAGTTGGATAGTTGTGGAAA
GAGTCAAATGGCTCTCCTCAAGCGTATTCAACAAGGGGCTGAAGGATGCC
CAGAAGGTACCCCATTGTATGGGATCTGATCTGGGGCCTCGGTGCACATG
CTTTACATGTGTTTAGTCGAGGTTAAAAAACGTCTAGGCCCCCCGAACCA
CGGGGACGTGGTTTTCCTTTGAAAAACACGATGATAATAGCCACCATGGG
AGTCAAAGTTCTGTTTGCCCTGATCTGCATCGCTGTGGCCGAGGCCAAGC
CCACCGAGAACAACGAAGACTTCAACATCGTGGCCGTGGCCAGCAACTTC
GCGACCACGGATCTCGATGCTGACCGCGGGAAGTTGCCCGGCAAGAAGCT
GCCGCTGGAGGTGCTCAAAGAGATGGAAGCCAATGCCCGGAAAGCTGGCT
GCACCAGGGGCTGTCTGATCTGCCTGTCCCACATCAAGTGCACGCCCAAG
ATGAAGAAGTTCATCCCAGGACGCTGCCACACCTACGAAGGCGACAAAGA
GTCCGCACAGGGCGGCATAGGCGAGGCGATCGTCGACATTCCTGAGATTC
CTGGGTTCAAGGACTTGGAGCCCATGGAGCAGTTCATCGCACAGGTCGAT
CTGTGTGTGGACTGCACAACTGGCTGCCTCAAAGGGCTTGCCAACGTGCA
GTGTTCTGACCTGCTCAAGAAGTGGCTGCCGCAACGCTGTGCGACCTTTG
CCAGCAAGATCCAGGGCCAGGTGGACAAGATCAAGGGGGCCGGTGGTGAC
TAAAAAAAACAAAAAACAAAACGGCTATT
Splint
SEQ ID NO: 23
GTTTTTCGGCTATTCCCAATAGCCGTTTTG

1. A method of producing human polyclonal antibodies, the method comprising the step of administering a circular polyribonucleotide comprising a sequence encoding an antigen to a non-human animal having a humanized immune system.

2. A method of inducing an immune response to an antigen, the method comprising the step of administering a circular polyribonucleotide comprising a sequence encoding the antigen to a non-human animal comprising a humanized immune system.

3. The method of any one of the preceding claims, wherein the antigen is from a microorganism, a cancer, or a toxin.

4. The method of any one of the preceding claims, wherein the antigen is from a pathogenic microorganism.

5. The method of any one of the preceding claims, wherein the antigen is from a virus or a fragment thereof, from a bacterium or a fragment thereof, from a eukaryotic parasite or a fragment thereof, or from a fungus or a fragment thereof.

6. The method of any one of the preceding claims, wherein the antigen is from a DNA virus or a fragment thereof, a positive strand RNA virus or a fragment thereof, or a negative strand RNA virus or a fragment thereof.

7. The method of any one of the preceding claims, wherein the antigen is from a virus selected from a group consisting of Marburg, ebola, rabies, HIV, smallpox, hantavirus, dengue, rotavirus, Crimean-Congo hemorrhagic fever, lassa fever, nipha and henipaviral disease, rift valley fever, plague, tularemia, machupo, typhus fever, CMV, Hepatitis B, Hepatitis C, HSV, parvovirus B19, rubella, zika, chickenpox, RSV, Para influenza, rhinovirus, adenovirus, metapneumovirus, bocavirus, community acquired respiratory virus, measles, mumps, and varicella, or any fragment thereof.

8. The method of any one of the preceding claims, wherein the antigen is selected from a coronavirus or a fragment thereof, a betacoronavirus or a fragment thereof, or a sarbecovirus or a fragment thereof.

9. The method of any one of the preceding claims, wherein the antigen is from severe acute respiratory syndrome-related coronavirus or a fragment thereof, a merbecovirus or a fragment thereof, or Middle East respiratory syndrome coronavirus (MERS-CoV) or a fragment thereof.

10. The method of any one of the preceding claims, wherein the antigen is from severe acute respiratory syndrome coronavirus 2 (SARS-CoV-2) or a fragment thereof or severe acute respiratory syndrome coronavirus 1 (SARS-CoV-1) or a fragment thereof.

11. The method of any one of the preceding claims, wherein the antigen is from a membrane protein of a virus or a variant or fragment thereof, an envelope protein of a virus or a variant or fragment thereof, a spike protein of a virus or a variant or fragment thereof, a receptor binding domain of a spike protein of a virus or a variant or fragment thereof, a nucleocapsid protein of a virus or a variant or fragment thereof, an accessory protein of a virus or a variant or fragment thereof.

12. The method of claim 11, wherein the spike protein lacks a cleavage site.

13. The method of any one of the preceding claims, wherein an accessory protein of a virus is selected from a group consisting of ORF3a, ORF7a, ORF7b, ORF8, ORF10, or any fragment thereof.

14. The method of any one of the preceding claims, wherein the antigen is a variant of an accessory protein of a virus selected from a group consisting of ORF3a, ORF7a, ORF7b, ORF8, and ORF10, or is a fragment thereof.

15. The method of any one of the preceding claims, wherein the antigen is from a bacterium selected from a group consisting of Group B strep, toxoplasma, and syphilis, or any fragment thereof.

16. The method of any one of the preceding claims, wherein the cancer antigen is HER2 or a cancer neoantigen.

17. The method of any one of the preceding claims, wherein the toxin antigen is from an animal venom, plant, or fungus.

18. The method of any one of the preceding claims, wherein the toxin antigen is from a drug (e.g., digoxin).

19. The method of any one of the preceding claims, wherein the circular polyribonucleotide comprises a sequence encoding two or more antigens or antigenic sequences.

20. The method of any one of the preceding claims, wherein the circular polyribonucleotide comprises two or more ORFs.

21. The method of any one of the preceding claims, further comprising administering and/or immunizing the non-human animal having the humanized immune system with a second circular polyribonucleotide comprising a sequence encoding a second antigen.

22. The method of any one of the preceding claims, further comprising administering and/or immunizing the non-human animal having the humanized immune system with a second circular polyribonucleotide comprising a second ORF.

23. The method of any one of the preceding claims, further comprising administering and/or immunizing the non-human animal having the humanized immune system with a third, fourth, or fifth circular polyribonucleotide comprising a sequence encoding a third, fourth, or fifth antigen or a third, fourth, or fifth antigenic sequence.

24. The method of any one of the preceding claims, wherein the non-human animal having a humanized immune system is a mammal.

25. The method of any one of the preceding claims, wherein the non-human animal having a humanized immune system is an ungulate.

26. The method of any one of the preceding claims, wherein the non-human animal having a humanized immune system is a transchromosomal ungulate.

27. The method of any one of the preceding claims, wherein the non-human animal having a humanized immune system is a cow or bovine.

28. The method of any one of the preceding claims, wherein the non-human animal having a humanized immune system comprises a human artificial chromosome (HAC) vector that comprises the humanized immunoglobulin gene locus.

29. The method of any one of the preceding claims, wherein the humanized immunoglobulin gene locus encodes an immunoglobulin heavy chain.

30. The method of any one of the preceding claims, wherein the humanized immunoglobulin gene locus encodes an immunoglobulin light chain.

31. The method of any one of the preceding claims, wherein the non-human animal having a humanized immune system comprises a B cell having a humanized B cell receptor, the humanized B cell receptor binds to the antigen.

32. The method of any one of the preceding claims, wherein the non-human animal having a humanized immune system comprises a plurality of B cells comprising a first B cell that binds to a first epitope of the antigen and a second B cell that binds to a second epitope of the antigen.

33. The method of any one of the preceding claims, wherein the non-human animal having a humanized immune system comprises a T cell, wherein the T cell comprises a T Cell Receptor that binds to the antigen.

34. The method of any one of the preceding claims, wherein upon activation, the T cell enhances production of an antibody that that binds to the antigen.

35. The method of any one of the preceding claims, wherein upon activation, the T cell enhances antibody production by a B cell that binds to the antigen.

36. The method of any one of the preceding claims, wherein upon activation, the T cell enhances survival, proliferation, plasma cell differentiation, somatic hypermutation, immunoglobulin class switching, or a combination thereof of a B cell that that binds to the antigen.

37. The method of any one of the preceding claims, wherein an antibody of the polyclonal antibodies specifically binds to the antigen or antigenic sequence.

38. The method of any one of the preceding claims, wherein an antibody of the polyclonal antibodies is a human antibody.

39. The method of any one of the preceding claims, wherein the polyclonal antibodies comprise fully human polyclonal antibodies.

40. The method of any one of the preceding claims, wherein the polyclonal antibodies comprise IgG antibodies, IgG1 antibodies, IgG2 antibodies, IgG3 antibodies, IgG4 antibodies, IgM antibodies, IgA antibodies, or a combination thereof.

41. The method of any one of the preceding claims, further comprising administering an adjuvant to the non-human animal having a humanized immune system.

42. The method of any one of the preceding claims, further comprising administering protamine to the non-human animal having a humanized immune system.

43. The method of any one of the preceding claims, further comprising administering the circular polyribonucleotide at least two times to the non-human animal having a humanized immune system to generate hyperimmune plasma.

44. The method of any one of the preceding claims, further comprising collecting plasma from the non-human animal having a humanized immune system.

45. The method of any one of the preceding claims, further comprising purifying polyclonal antibodies from the plasma of a non-human animal having a humanized immune system.

46. The method of any one of the preceding claims, further comprising administering a second agent or a vaccine to the non-human animal having a humanized immune system.

47. The method of any one of the preceding claims, wherein the vaccine is pneumococcal polysaccharide vaccine (e.g., PCV13 or PPSV23).

48. The method of any one of the preceding claims, wherein the vaccine is for a bacterial infection.

49. The method of any one of the preceding claims, further comprising administering the non-human animal having a humanized immune system with the antigen prior to administration of the circular polyribonucleotide.

50. The method of any one of the preceding claims, further comprising administering the antigen to the non-human animal having a humanized immune system at least 1, 2, 3, 4, 5, 6, or 7 days prior to administering the circular polyribonucleotide.

51. A method of producing a human polyclonal antibody preparation against a target, comprising:
a) administering to a non-human animal capable of producing human antibodies an immunogenic composition comprising a circular that comprises a sequence encoding an antigen of the target,
b) collecting blood or plasma from the non-human animal capable of producing human antibodies,
c) purifying antibodies against the antigen from the blood or plasma, and
d) formulating the antibodies as a therapeutic or pharmaceutical preparation for human use.

52. The method of claim 51, wherein the target a microorganism, a cancer, or a toxin.
